SEQUENCE LISTING (115 sequences)

SEQ ID NO: 1 - protein, 329 aa, Homo sapiens
  MISC_FEATURE: IgG1 heavy chain constant region protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 329 aa, Homo sapiens
  MISC_FEATURE: C220S IgG1 heavy constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 328 aa, Homo sapiens
  MISC_FEATURE: C220delta IgG1 heavy constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 107 aa, Homo sapiens [sequence omitted]

SEQ ID NO: 5 - protein, 107 aa, Homo sapiens
  MISC_FEATURE: C214S Kappa light chain constant region
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 6 - protein, 106 aa, Homo sapiens
  MISC_FEATURE: C214delta Kappa light chain constant region
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 7 - protein, 105 aa, Homo sapiens
  MISC_FEATURE: Lambda light chain constant region
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 8 - protein, 105 aa, Homo sapiens
  MISC_FEATURE: C214S Lambda light chain constant region
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTESS

SEQ ID NO: 9 - protein, 104 aa, Homo sapiens
  MISC_FEATURE: C214delta Lambda light chain constant region
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTES

SEQ ID NO: 10 - protein, 220 aa, Homo sapiens
  MISC_FEATURE: Protein sequence of CLDN6 (NP_067018.2)
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 11 - protein, 217 aa, Homo sapiens
  MISC_FEATURE: Protein sequence of CLDN9 (NP_066192.1)
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 12-19 - skipped (8 entries)

SEQ ID NO: 20 - DNA, 321 bp, Mus musculus
  misc_feature: SC27.1 Light Chain Variable Region
gacatccagatgacacaatcttcatcctccttttctgtatctctaggagacagagtcaccattacttgcaaggcaagtgaagacatatataatcggttagcctggtatcagcagaaaccaggaaatgctcccaggctcttaatatctggtgcaaccagtttggaaactgggactccttcaagattcagtggcagtggatctggaaaggattacactctcagtattaccagtcttcggactgaagatgctgctacttattactgtcaacaatattggagtactccactcacgttcggtactgggaccaagctggagctgaaa

SEQ ID NO: 21 - protein, 107 aa, Mus musculus
  MISC_FEATURE: SC27.1 Light Chain Variable Region
DIQMTQSSSSFSVSLGDRVTITCKASEDIYNRLAWYQQKPGNAPRLLISGATSLETGTPSRFSGSGSGKDYTLSITSLRTEDAATYYCQQYWSTPLTFGTGTKLELK

SEQ ID NO: 22 - DNA, 357 bp, Mus musculus
  misc_feature: SC27.1 Heavy Chain Variable Region
gaggtccagctgcaagagtctagacctgagctggtgaagcctggggcttcagtgaagatatcctgcaagacttctggatacacattcactgaatacaccttgcactgggtgaagcagagtcatggaaagagccttgagtggattggaggtattaatcctaacaatggtgatactatctacaaccagaaattcaagggcaaggccacattgactgtagacaagtcctccagcacagcctacatggagctccgcagcctgacatctgaatattctgcagtctattactgtgcaagaagggcgattacggtctatgctatggactactggggtcaaggtacctcagtcaccgtctcctca

SEQ ID NO: 23 - protein, 119 aa, Mus musculus
  MISC_FEATURE: SC27.1 Heavy Chain Variable Region
EVQLQESRPELVKPGASVKISCKTSGYTFTEYTLHWVKQSHGKSLEWIGGINPNNGDTIYNQKFKGKATLTVDKSSSTAYMELRSLTSEYSAVYYCARRAITVYAMDYWGQGTSVTVSS

SEQ ID NO: 24 - DNA, 333 bp, Mus musculus
  misc_feature: SC27.22 Light Chain Variable Region
gacattgtgctgacacagtctcctgcttccttagctgtatctctggggcagagggccaccatctcatgcagggccagccagactgtcagtacatctagctatagttatatgcactggttccaacagaaaccaggacagccacccaaactcctcatcaagtttgcatccaacgtagaatctggggtccctgccagattcagtggcagtgggtctgggacagacttcaccctcaacatccatcctgtggaggaggaggatatttcaacatattactgtcagcacagttgggagattccgtggacgttcggtggaggcaccaagctggaaatcaaa

SEQ ID NO: 25 - protein, 111 aa, Mus musculus
  MISC_FEATURE: SC27.22 Light Chain Variable Region
DIVLTQSPASLAVSLGQRATISCRASQTVSTSSYSYMHWFQQKPGQPPKLLIKFASNVESGVPARFSGSGSGTDFTLNIHPVEEEDISTYYCQHSWEIPWTFGGGTKLEIK

SEQ ID NO: 26 - DNA, 366 bp, Mus musculus
  misc_feature: SC27.22 Heavy Chain Variable Region
caggtccaactgcagcagcctggggctgagctggtgaggcctggagcttcagtgaagctgtcctgcaaggcttctggctacaccttcaccagctactggatgaactgggtgaagcagaggcctggacaaggccttgaatggattgccatgattcatccttccgatagtgaaattaggttaaatcagaagttcaaggacaaggccacattgactgtagacagatcctccagcacagcctacatgcaactcagcagcccgacatctgaggactctgcggtctattactgtgcaagaattgatagttattatggttacctgttttactttgactactggggccaaggcaccactctcacagtctcctca

SEQ ID NO: 27 - protein, 122 aa, Mus musculus
  MISC_FEATURE: SC27.22 Heavy Chain Variable Region
QVQLQQPGAELVRPGASVKLSCKASGYTFTSYWMNWVKQRPGQGLEWIAMIHPSDSEIRLNQKFKDKATLTVDRSSSTAYMQLSSPTSEDSAVYYCARIDSYYGYLFYFDYWGQGTTLTVSS

SEQ ID NO: 28 - DNA, 324 bp, Mus musculus
  misc_feature: SC27.103 Light Chain Variable Region
caaattgttctcacccagtctccagcaatcatgtctgcatctctaggggaacgggtcaccatgacctgcactgccagctcaagtgtaagttccagttacttgcactggtaccagcagaagccaggatcctcccccacactctggatttataggacatccgacctggcttctggagtcccagctcgcttcagtggcagtggatctgggacctcttactctctcacaatcagcagcatggaggctgaagatgctgccacttattactgccaccagtatcatcgttccccgtggacgttcggtggaggcaccaggctggaaatcaaa

SEQ ID NO: 29 - protein, 108 aa, Mus musculus
  MISC_FEATURE: SC27.103 Light Chain Variable Region
QIVLTQSPAIMSASLGERVTMTCTASSSVSSSYLHWYQQKPGSSPTLWIYRTSDLASGVPARFSGSGSGTSYSLTISSMEAEDAATYYCHQYHRSPWTFGGGTRLEIK

SEQ ID NO: 30 - DNA, 354 bp, Mus musculus
  misc_feature: SC27.103 Heavy Chain Variable Region
gaggtccacctgcaacagtctggacctgagctagtgaagcctggaggttcaatgaagatatcctgcaaggcttctggttactcattcactggctacaccatgaactgggtgaagcagagccatggaaagaaccttgagtggattggactttttaatccttacaatggtggtactagttataaccagaagttcaagggcaaggccacattaactgtagacaagtcatccagcacagcctacatggagctcctcagtctgacatctgaggactctgcagtctattactgtgcaagatgctataggtacgacggtcttgactactggggccaaggcaccactctcacagtctcctca

SEQ ID NO: 31 - protein, 118 aa, Mus musculus
  MISC_FEATURE: SC27.103 Heavy Chain Variable Region
EVHLQQSGPELVKPGGSMKISCKASGYSFTGYTMNWVKQSHGKNLEWIGLFNPYNGGTSYNQKFKGKATLTVDKSSSTAYMELLSLTSEDSAVYYCARCYRYDGLDYWGQGTTLTVSS

SEQ ID NO: 32 - DNA, 321 bp, Mus musculus
  misc_feature: SC27.104 Light Chain Variable Region
gacatccagatgacacaatcttcatcctccttttctgtatctctaggagacagagtcaccattacttgcaaggcaagtgaggacatatataatcggttagcctggtatcagcagaaaccaggaaatgctcccaggctcttaatatctggtgcaaccagtttggaaactggggttccttcaagattcagtggcagtggatctggaaaggattacactctcagcattaccagtcttcagactgaagatgttgctacttattactgtcaacagtattggagtaatcctccgacgttcggtggaggcaccaagctggaaatcaaa

SEQ ID NO: 33 - protein, 107 aa, Mus musculus
  MISC_FEATURE: SC27.104 Light Chain Variable Region
DIQMTQSSSSFSVSLGDRVTITCKASEDIYNRLAWYQQKPGNAPRLLISGATSLETGVPSRFSGSGSGKDYTLSITSLQTEDVATYYCQQYWSNPPTFGGGTKLEIK

SEQ ID NO: 34 - DNA, 354 bp, Mus musculus
  misc_feature: SC27.104 Heavy Chain Variable Region
gaggtccagctgcaacagtctggacctgagctggtgaagcctggggcttcagtgaagatatcctgcaagacttctggatacacattcactgaatacaccgtgcactgggtgaagcagagccatggaaagagccttgagtggattggaggtgtttatcctaagaatggtgatactacctacaaccagaagttcaggggcaaggccacattgactgtagacaagtcctccaacacagcctatatggaactccgcagcctgacatctgaggattctgcagtctattactgtacaggaaaggatgggtacgacgggtttgcttactggggccaagggactctggtcactgtctctgca

SEQ ID NO: 35 - protein, 118 aa, Mus musculus
  MISC_FEATURE: SC27.104 Heavy Chain Variable Region
EVQLQQSGPELVKPGASVKISCKTSGYTFTEYTVHWVKQSHGKSLEWIGGVYPKNGDTTYNQKFRGKATLTVDKSSNTAYMELRSLTSEDSAVYYCTGKDGYDGFAYWGQGTLVTVSA

SEQ ID NO: 36 - DNA, 318 bp, Mus musculus
  misc_feature: SC27.105 Light Chain Variable Region
gatgttcaaatgacccagtctccatcctccctgtctgcatctttgggagagagagtctccctgacttgccaggcaagtcagagtgttagcaataatttaaactggtatcagcaaacaccagggaaagctcctaggctcttgatctatggtgcaagcaaattggaagatggggtctcttcaaggttcagtggcactggatatgggacagatttcactttcaccatcagcagcctggaggaagaagatgtggcaacttatttttgtctacagcataggtatctgtggacgttcggtggaggcaccaagctggaaatcaaa

SEQ ID NO: 37 - protein, 106 aa, Mus musculus
  MISC_FEATURE: SC27.105 Light Chain Variable Region
DVQMTQSPSSLSASLGERVSLTCQASQSVSNNLNWYQQTPGKAPRLLIYGASKLEDGVSSRFSGTGYGTDFTFTISSLEEEDVATYFCLQHRYLWTFGGGTKLEIK

SEQ ID NO: 38 - DNA, 369 bp, Mus musculus
  misc_feature: SC27.105 Heavy Chain Variable Region
gaggtccagctgcagcagtctggacctgagttggtgaagcctggggcttcagtgaagatatcctgcaaggcttctggttactcattcactggctactacatgaactgggtgaagcaaagtcctgaaaagagccttgagtggattggagagattaatcctagcactggtagtactacttacaaccagaagttcaaggccaaggccacattgactgtagacaaatcctccagcacagcctacatgcagctcaagagcctgacatctgaggactctgcagtctattactgtgcaagaagggattattactacggtagtggtttctatgctatggactactggggtcaaggaacctcagtcaccgtctcctca

SEQ ID NO: 39 - protein, 123 aa, Mus musculus
  MISC_FEATURE: SC27.105 Heavy Chain Variable Region
EVQLQQSGPELVKPGASVKISCKASGYSFTGYYMNWVKQSPEKSLEWIGEINPSTGSTTYNQKFKAKATLTVDKSSSTAYMQLKSLTSEDSAVYYCARRDYYYGSGFYAMDYWGQGTSVTVSS

SEQ ID NO: 40 - DNA, 321 bp, Mus musculus
  misc_feature: SC27.106 Light Chain Variable Region
gacatccagatgacacaatcttcatcctccttttctgtatctctaggagacagagtcaccattacttgcaaggcaagtgaggacatatataatcggttagcctggtatcagcagaaaccaggaaatgctcctaggctcttaatatgtggtgcaaccagtttggaaactggggttccttcaagattcagtggcagtggatctggaaaggattacactctcagcattaccagtcttcagactgaagatgttgctacttattactgtcaacagtattggagtactccgctcacgttcggtgctgggaccaaactggagctgaaa

SEQ ID NO: 41 - protein, 107 aa, Mus musculus
  MISC_FEATURE: SC27.106 Light Chain Variable Region
DIQMTQSSSSFSVSLGDRVTITCKASEDIYNRLAWYQQKPGNAPRLLICGATSLETGVPSRFSGSGSGKDYTLSITSLQTEDVATYYCQQYWSTPLTFGAGTKLELK

SEQ ID NO: 42 - DNA, 357 bp, Mus musculus
  misc_feature: SC27.106 Heavy Chain Variable Region
gaggtccagctgcaacagtctggacctgagctggtgaagcctggggcttcagtgaagatatcctgcaagacttctggatacacattcactgaatacaccatgcactgggtgaagcagagccatggaaagagccttgagtggattggaggtattaatcctaacaatggtggtactaactacaaccagaagttcaagggcaaggccacattgactgttgacaagtcctccagcacagcctacatggagctccgcagcctgacatctgaggattctgcagtctattactgtgcaagaaggcttattacttactatgctatggactactggggtcaaggaacctcagtcaccgtctcctca

SEQ ID NO: 43 - protein, 119 aa, Mus musculus
  MISC_FEATURE: SC27.106 Heavy Chain Variable Region
EVQLQQSGPELVKPGASVKISCKTSGYTFTEYTMHWVKQSHGKSLEWIGGINPNNGGTNYNQKFKGKATLTVDKSSSTAYMELRSLTSEDSAVYYCARRLITYYAMDYWGQGTSVTVSS

SEQ ID NO: 44 - DNA, 324 bp, Mus musculus
  misc_feature: SC27.108 Light Chain Variable Region
gaaattgtgctcacccagtctccagcactcatggctgcatctccaggggagaaggtcaccatcacctgcagtgtcagctcaagtataagttccagcaacttgcactggtaccagcagaagtcaggaacctcccccaaactctggatttatggcacatccaacctggcttctggagtccctgttcgcttcagtggcagtggatctgggacctcttattctctcacaatcagcaacatggaggctgaagatgctgccacttattactgtcaacagtggagtagttacccacacacgttcggaggggggaccaagctggaaataaaa

SEQ ID NO: 45 - protein, 108 aa, Mus musculus
  MISC_FEATURE: SC27.108 Light Chain Variable Region
EIVLTQSPALMAASPGEKVTITCSVSSSISSSNLHWYQQKSGTSPKLWIYGTSNLASGVPVRFSGSGSGTSYSLTISNMEAEDAATYYCQQWSSYPHTFGGGTKLEIK

SEQ ID NO: 46 - DNA, 378 bp, Mus musculus
  misc_feature: SC27.108 Heavy Chain Variable Region
caggtccaaatgcagcagtctggagctgagctggtaaggcctgggacttcagtgaaggtgtcctgcaaggcttctggatacgccttcactaattacttgatagagtgggtaaagcagaggcctggacagggccttgagtggattggactgattaatcctggaagtggtggtactaattacaatgagaagttcaagggcaaggcaacactgactgcagacaaatcctccaccactgcctacatgcagctcagcagcctgacatctgatgactctgcggtttatttctgtgcaagacggtcccctctagggagttggatctactatgcttacgacggtgttgcttactggggccaagggactctggtcactgtctctgca

SEQ ID NO: 47 - protein, 126 aa, Mus musculus
  MISC_FEATURE: SC27.108 Heavy Chain Variable Region
QVQMQQSGAELVRPGTSVKVSCKASGYAFTNYLIEWVKQRPGQGLEWIGLINPGSGGTNYNEKFKGKATLTADKSSTTAYMQLSSLTSDDSAVYFCARRSPLGSWIYYAYDGVAYWGQGTLVTVSA

SEQ ID NO: 48 - DNA, 321 bp, Mus musculus
  misc_feature: SC27.201 Light Chain Variable Region
gacatccagatgacacaatcttcatcctccttttctgtctctctgggagacagagtcactattacttgcaaggcaagtgaggacatctataatcggttagcctggtatcaacagaaaccaggaaatgctcctaggctcttaatatctggtgcaaccagtttggaagctggggttccttcaggattcagtggcagtggatctggaaaggattacactctcagcattaccagtcttcagactgaagatgttgctacttattactgtcaacagtattggagtactcctccgacgttcggtggaggcaccaagctggaactcaag

SEQ ID NO: 49 - protein, 107 aa, Mus musculus
  MISC_FEATURE: SC27.201 Light Chain Variable Region
DIQMTQSSSSFSVSLGDRVTITCKASEDIYNRLAWYQQKPGNAPRLLISGATSLEAGVPSGFSGSGSGKDYTLSITSLQTEDVATYYCQQYWSTPPTFGGGTKLELK

SEQ ID NO: 50 - DNA, 360 bp, Mus musculus
  misc_feature: SC27.201 Heavy Chain Variable Region
gaggtccagctgcaacagtctggacctgaactggtgaagcctggggcttcagtgaagatatcctgcaagacttctggatacacattcactgaaaacatcagacactgggtgaagcagagccgaggaaagagccttgagtggattggtactattaatcctaataatggtgagactaggtacaatcagaagttcaagggcaaggccacattgactgtagacaagtcctccagcacagcctacatggagctccgcagcctgacatctgaggattctgcagtctattactgtacaagggggattacaaagtccccttatggtatggactactggggtcaaggaacctcaatcaccgtctcctca

SEQ ID NO: 51 - protein, 120 aa, Mus musculus
  MISC_FEATURE: SC27.201 Heavy Chain Variable Region
EVQLQQSGPELVKPGASVKISCKTSGYTFTENIRHWVKQSRGKSLEWIGTINPNNGETRYNQKFKGKATLTVDKSSSTAYMELRSLTSEDSAVYYCTRGITKSPYGMDYWGQGTSITVSS

SEQ ID NO: 52 - DNA, 321 bp, Mus musculus
  misc_feature: SC27.203 Light Chain Variable Region
gacatccagatgacacaatcttcatcctccttttctgtatctctaggagacagagtcaccatcacttgcaaggcaagtgaggacatatataatcggttagcctggtatcagcagaatccaggaaatactcctaggctcttaatgtctggtgcaaccagtttggaaactggggttccttcaagattcagtggcagtggatctggaaaggattacactctcagcattaccagtcttcagattgaagatgtttctacttattactgtcaacaatattggagtactcctccgacgttcggtggaggcaccaggctggaaatcaaa

SEQ ID NO: 53 - protein, 107 aa, Mus musculus
  MISC_FEATURE: SC27.203 Light Chain Variable Region
DIQMTQSSSSFSVSLGDRVTITCKASEDIYNRLAWYQQNPGNTPRLLMSGATSLETGVPSRFSGSGSGKDYTLSITSLQIEDVSTYYCQQYWSTPPTFGGGTRLEIK

SEQ ID NO: 54 - DNA, 360 bp, Mus musculus
  misc_feature: SC27.203 Heavy Chain Variable Region
gaggtccagctgcaacagtctggacctgagctggtgaagcctggggcttcagtgaagatatcctgcaagacttctggatacacattcactgaaaacatcatacactgggtgaagcagagccatggaaagagccttgagtggattggaggtattaatcctatcaatggtggtactagctacaaccagaagttcaagggcaaggccacattgactgtagacaagtcctccagcacagcctacatggagctccgtagcctgacatctgaggattctgcagtctattactgtgcaagggggattactacgtccccttatgctatggactactggggtcaaggaacctcagtcaccgtctcctca

SEQ ID NO: 55 - protein, 120 aa, Mus musculus
  MISC_FEATURE: SC27.203 Heavy Chain Variable Region
EVQLQQSGPELVKPGASVKISCKTSGYTFTENIIHWVKQSHGKSLEWIGGINPINGGTSYNQKFKGKATLTVDKSSSTAYMELRSLTSEDSAVYYCARGITTSPYAMDYWGQGTSVTVSS

SEQ ID NO: 56 - DNA, 321 bp, Mus musculus
  misc_feature: SC27.204 Light Chain Variable Region
gacattgtgatgacccagtctcaaaaattcatgtccacatcagtaggagacagggtcagcgtcgcctgcaaggccggtcagaatgtgggtactagtgtagcctggtatcaacagaaaccaggacattctcctaaatcactgatttactcggcatcctaccggtacagtggagtccctaatcgcttcacaggcagtggatctgggacagatttcactctcaccatcagcaatgtgcagtctgaagacttggcagactatttctgtcagcaatatatcacctatccgtacacgttcggaggggggaccaagctggaaataata

SEQ ID NO: 57 - protein, 107 aa, Mus musculus
  MISC_FEATURE: SC27.204 Light Chain Variable Region
DIVMTQSQKFMSTSVGDRVSVACKAGQNVGTSVAWYQQKPGHSPKSLIYSASYRYSGVPNRFTGSGSGTDFTLTISNVQSEDLADYFCQQYITYPYTFGGGTKLEII

SEQ ID NO: 58 - DNA, 336 bp, Mus musculus
  misc_feature: SC27.204 Heavy Chain Variable Region
gaggtgaaggttctcgagtctggaggtggcctggtgcagcctggaggatccctgaaactctcctgtgcagcctcaggattcgattttagtagatactggatgagttgggtccggcaggctccagggaaaggcctagaatggattggagaaattaatccagatagcagtacgataaactatacgccatctctaaaggctaaattcatcatctccagagacaacgccaaaaatacgctgtacctgcaaatgagcaaagtgagatctgaggacacagccctttattactgtacaggaccagcttactggggccaagggactctggtcactgtctctgca

SEQ ID NO: 59 - protein, 112 aa, Mus musculus
  MISC_FEATURE: SC27.204 Heavy Chain Variable Region
EVKVLESGGGLVQPGGSLKLSCAASGFDFSRYWMSWVRQAPGKGLEWIGEINPDSSTINYTPSLKAKFIISRDNAKNTLYLQMSKVRSEDTALYYCTGPAYWGQGTLVTVSA

SEQ ID NO: 60 - DNA, 321 bp, Artificial Sequence
  hSC27.1 Light Chain Variable Region
gacatccagatgacccagtctccatcttccgtgtctgcatctgtaggagacagagtcaccatcacttgtaaggcgagtgaggatatttacaaccggttagcctggtatcagcagaaaccagggaaagcccctaagctcctgatctatggtgcaaccagtttggaaactggggtcccatcaaggttcagcggcagtggatctgggacagattacactctcaccatcagcagcctgcagcctgaagattttgcaacttactattgtcaacagtattggagtactccactcacgttcggtcaagggaccaagctggagattaaa

SEQ ID NO: 61 - protein, 107 aa, Artificial Sequence
  hSC27.1 Light Chain Variable Region
DIQMTQSPSSVSASVGDRVTITCKASEDIYNRLAWYQQKPGKAPKLLIYGATSLETGVPSRFSGSGSGTDYTLTISSLQPEDFATYYCQQYWSTPLTFGQGTKLEIK

SEQ ID NO: 62 - DNA, 357 bp, Artificial Sequence
  hSC27.1 Heavy Chain Variable Region
caggtccagcttgtgcagtctggggctgaggtgaagaagcctggggcctcagtgaaggtttcctgcaaggcttctggatacaccttcactgagtatactctgcattgggtgcgccaggcccccggacaaaggcttgagtggatgggagggatcaaccctaacaatggtgacacaatatataaccagaagttcaagggcagagtcaccattaccagggacacatccgcgagcacagcctacatggagctgagcagcctgagatctgaagacacggctgtgtattactgtgcgagaagggcgattacggtctatgctatggactactggggtcaaggtaccctagtcaccgtctcgagc

SEQ ID NO: 63 - protein, 119 aa, Artificial Sequence
  hSC27.1 Heavy Chain Variable Region
QVQLVQSGAEVKKPGASVKVSCKASGYTFTEYTLHWVRQAPGQRLEWMGGINPNNGDTIYNQKFKGRVTITRDTSASTAYMELSSLRSEDTAVYYCARRAITVYAMDYWGQGTLVTVSS

SEQ ID NO: 64 - DNA, 333 bp, Artificial Sequence
  hSC27.22 Light Chain Variable Region
gacattgtcatgacccagtcccctgacagtttggccgttagcttgggggagcgtgccaccatcaactgtagggctagtcaaactgtttctacatcctcctactcttacatgcattggtatcagcagaaacctggtcagcctccaaaactgctgatttatttcgcatctaacgtcgagtccggagttcctgaccggttcagcggatcaggaagcggtacagattttacacttaccatctcatctctgcaagcagaagatgtggccgtgtactattgtcagcattcctgggagatcccctggaccttcgggcagggaaccaagctcgagattaaa

SEQ ID NO: 65 - protein, 111 aa, Artificial Sequence
  hSC27.22 Light Chain Variable Region
DIVMTQSPDSLAVSLGERATINCRASQTVSTSSYSYMHWYQQKPGQPPKLLIYFASNVESGVPDRFSGSGSGTDFTLTISSLQAEDVAVYYCQHSWEIPWTFGQGTKLEIK

SEQ ID NO: 66 - DNA, 366 bp, Artificial Sequence
  hSC27.22 Heavy Chain Variable Region
caggtgcagttggtgcagagcggcgccgaagtcaagaaaccaggagcttctgtcaaagtctcctgtaaagcctccggatataccttcaccagctactggatgaattgggtaagacaggcccccggacagaggcttgagtggatgggaatgatccatccctctgacagcgagattcggctcaaccagaagtttaaagaccgagtgactatcacacgcgataccagtgctagcacagcctacatggagttgagttctcttcgtagcgaggacactgccgtgtattattgcgcccgcatcgactcatattatggttatctgttctacttcgactattggggccaggggaccaccgtgactgtgtcttcc

SEQ ID NO: 67 - protein, 122 aa, Artificial Sequence
  hSC27.22 Heavy Chain Variable Region
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSS

SEQ ID NO: 68 - DNA, 324 bp, Artificial Sequence
  hSC27.108 Light Chain Variable Region
gaaatcgtgcttacacaatcccctgccactctgagcctttctccaggcgagcgagcaaccctttcctgcagtgtttcctcttcaatcagttccagcaatttgcactggtaccagcagaagcctggtcaggcaccccgattgttgatctatggcacatctaacctggccagcggcatccctgctcggttcagtggatctggctccggaacagatttcactctcactatcagctcccttgagcctgaagattttgccgtgtactactgtcagcaatggagttcctacccccacacctttggcggcgggacaaaggtcgagataaaa

SEQ ID NO: 69 - protein, 108 aa, Artificial Sequence
  hSC27.108 Light Chain Variable Region
EIVLTQSPATLSLSPGERATLSCSVSSSISSSNLHWYQQKPGQAPRLLIYGTSNLASGIPARFSGSGSGTDFTLTISSLEPEDFAVYYCQQWSSYPHTFGGGTKVEIK

SEQ ID NO: 70 - DNA, 378 bp, Artificial Sequence
  hSC27.108 Heavy Chain Variable Region
caggtacagctggtccagtccggcgctgaggttaagaagcccggtgcctccgtgaaggtatcttgtaaggcctcaggttacacctttacaaattatctgatcgaatgggtgagacaggccccaggtcagggtctggaatggatgggactcatcaaccctgggagtggcgggaccaactacaacgaaaagtttaaggggagagtgacaatgaccacagataccagtacctccaccgcatatatggagctgcgaagcttgaggtccgatgacactgctgtgtactattgcgcccgtagaagcccactcgggtcttggatctattacgcatacgatggtgtggcctattggggccagggcaccctggtgacagtcagctcc

SEQ ID NO: 71 - protein, 126 aa, Artificial Sequence
  hSC27.108 Heavy Chain Variable Region
QVQLVQSGAEVKKPGASVKVSCKASGYTFTNYLIEWVRQAPGQGLEWMGLINPGSGGTNYNEKFKGRVTMTTDTSTSTAYMELRSLRSDDTAVYYCARRSPLGSWIYYAYDGVAYWGQGTLVTVSS

SEQ ID NO: 72 - DNA, 321 bp, Artificial Sequence
  hSC27.204 Light Chain Variable Region
gacatccagatgacccagtccccctccagcctgtctgcttccgtgggcgacagagtgaccatcacatgcaaggccggccagaacgtgggcacctctgtggcctggttccagcagaagcctggcaaggcccccaagtccctgatctactccgcctcctacagatactccggcgtgccctccagattctccggctctggctctggcaccgactttaccctgaccatcagctccctgcagcccgaggacttcgccacctactactgccagcagtacatcacctacccctacaccttcggcggaggcaccaaggtggaaatcaag

SEQ ID NO: 73 - protein, 107 aa, Artificial Sequence
  hSC27.204 Light Chain Variable Region
DIQMTQSPSSLSASVGDRVTITCKAGQNVGTSVAWFQQKPGKAPKSLIYSASYRYSGVPSRFSGSGSGTDFTLTISSLQPEDFATYYCQQYITYPYTFGGGTKVEIK

SEQ ID NO: 74 - DNA, 336 bp, Artificial Sequence
  hSC27.204 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatcaactacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 75 - protein, 112 aa, Artificial Sequence
  hSC27.204 Heavy Chain Variable Region
EVQLLESGGGLVQPGGSLRLSCAASGFTFSRYWMSWVRQAPGKGLEWVSEINPDSSTINYTPSLKARFTISRDNSKNTLYLQMNSLRAEDTAVYYCTGPAYWGQGTLVTVSS

SEQ ID NO: 76 - DNA, 336 bp, Artificial Sequence
  hSC27.204v2 Heavy Chain Variable Region
gaagtgcagctgctggaatctggcggcggactggtgcagcctggcggatctctgagactgtcttgtgccgcctccggcttcaccttctcccggtactggatgtcctgggtgcgacaggctcctggcaagggcctggaatgggtgtccgagatcaaccccgactcctccaccatccagtacacccccagcctgaaggcccggttcaccatctctcgggacaactccaagaacaccctgtacctgcagatgaactccctgcgggccgaggacaccgccgtgtactactgtaccggccctgcttattggggccagggcaccctcgtgaccgtgtcctct

SEQ ID NO: 77 - protein, 112 aa, Artificial Sequence
  hSC27.204v2 Heavy Chain Variable Region
EVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCTGPAYWGQGTLVTVSS

SEQ ID NO: 78 - protein, 214 aa, Artificial Sequence
  hSC27.1 Light Chain
DIQMTQSPSSVSASVGDRVTITCKASEDIYNRLAWYQQKPGKAPKLLIYGATSLETGVPSRFSGSGSGTDYTLTISSLQPEDFATYYCQQYWSTPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 448 aa, Artificial Sequence
  hSC27.1 Heavy Chain
QVQLVQSGAEVKKPGASVKVSCKASGYTFTEYTLHWVRQAPGQRLEWMGGINPNNGDTIYNQKFKGRVTITRDTSASTAYMELSSLRSEDTAVYYCARRAITVYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 218 aa, Artificial Sequence
  hSC27.22 Light Chain
DIVMTQSPDSLAVSLGERATINCRASQTVSTSSYSYMHWYQQKPGQPPKLLIYFASNVESGVPDRFSGSGSGTDFTLTISSLQAEDVAVYYCQHSWEIPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 451 aa, Artificial Sequence
  hSC27.22 Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 451 aa, Artificial Sequence
  hSC27.22ss1 Heavy Chain
QVQLVQSGAEVKKPGASVKVSCKASGYTFTSYWMNWVRQAPGQRLEWMGMIHPSDSEIRLNQKFKDRVTITRDTSASTAYMELSSLRSEDTAVYYCARIDSYYGYLFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 215 aa, Artificial Sequence
  hSC27.108 Light Chain
EIVLTQSPATLSLSPGERATLSCSVSSSISSSNLHWYQQKPGQAPRLLIYGTSNLASGIPARFSGSGSGTDFTLTISSLEPEDFAVYYCQQWSSYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 455 aa, Artificial Sequence
  hSC27.108 Heavy Chain
QVQLVQSGAEVKKPGASVKVSCKASGYTFTNYLIEWVRQAPGQGLEWMGLINPGSGGTNYNEKFKGRVTMTTDTSTSTAYMELRSLRSDDTAVYYCARRSPLGSWIYYAYDGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 455 aa, Artificial Sequence
  hSC27.108ss1 Heavy Chain
QVQLVQSGAEVKKPGASVKVSCKASGYTFTNYLIEWVRQAPGQGLEWMGLINPGSGGTNYNEKFKGRVTMTTDTSTSTAYMELRSLRSDDTAVYYCARRSPLGSWIYYAYDGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 214 aa, Artificial Sequence
  hSC27.204 Light Chain
DIQMTQSPSSLSASVGDRVTITCKAGQNVGTSVAWFQQKPGKAPKSLIYSASYRYSGVPSRFSGSGSGTDFTLTISSLQPEDFATYYCQQYITYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 441 aa, Artificial Sequence
  hSC27.204 Heavy Chain
EVQLLESGGGLVQPGGSLRLSCAASGFTFSRYWMSWVRQAPGKGLEWVSEINPDSSTINYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 441 aa, Artificial Sequence
  hSC27.204v2 Heavy Chain
EVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 441 aa, Artificial Sequence
  hSC27.204v2ss1 Heavy Chain
EVQLLESGGGLVQPGGSLRLSCAASGFTFSRYWMSWVRQAPGKGLEWVSEINPDSSTIQYTPSLKARFTISRDNSKNTLYLQMNSLRAEDTAVYYCTGPAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 11 aa, Artificial Sequence
  hSC27.1 CDRL1
KASEDIYNRLA

SEQ ID NO: 91 - protein, 7 aa, Artificial Sequence
  hSC27.1 CDRL2
GATSLET

SEQ ID NO: 92 - protein, 9 aa, Artificial Sequence
  hSC27.1 CDRL3
QQYWSTPLT

SEQ ID NO: 93 - protein, 5 aa, Artificial Sequence
  hSC27.1 CDRH1
EYTLH

SEQ ID NO: 94 - protein, 17 aa, Artificial Sequence
  hSC27.1 CDRH2
GINPNNGDTIYNQKFKG

SEQ ID NO: 95 - protein, 10 aa, Artificial Sequence
  hSC27.1 CDRH3
RAITVYAMDY

SEQ ID NO: 96 - protein, 15 aa, Artificial Sequence
  hSC27.22 CDRL1
RASQTVSTSSYSYMH

SEQ ID NO: 97 - protein, 7 aa, Artificial Sequence
  hSC27.22 CDRL2
FASNVES

SEQ ID NO: 98 - protein, 9 aa, Artificial Sequence
  hSC27.22 CDRL3
QHSWEIPWT

SEQ ID NO: 99 - protein, 5 aa, Artificial Sequence
  hSC27.22 CDRH1
SYWMN

SEQ ID NO: 100 - protein, 17 aa, Artificial Sequence
  hSC27.22 CDRH2
MIHPSDSEIRLNQKFKD

SEQ ID NO: 101 - protein, 13 aa, Artificial Sequence
  hSC27.22 CDRH3
IDSYYGYLFYFDY

SEQ ID NO: 102 - protein, 12 aa, Artificial Sequence
  hSC27.108 CDRL1
SVSSSISSSNLH

SEQ ID NO: 103 - protein, 7 aa, Artificial Sequence
  hSC27.108 CDRL2
GTSNLAS

SEQ ID NO: 104 - protein, 9 aa, Artificial Sequence
  hSC27.108 CDRL3
QQWSSYPHT

SEQ ID NO: 105 - protein, 5 aa, Artificial Sequence
  hSC27.108 CDRH1
NYLIE

SEQ ID NO: 106 - protein, 17 aa, Artificial Sequence
  hSC27.108 CDRH2
LINPGSGGTNYNEKFKG

SEQ ID NO: 107 - protein, 17 aa, Artificial Sequence
  hSC27.108 CDRH3
RSPLGSWIYYAYDGVAY

SEQ ID NO: 108 - skipped

SEQ ID NO: 109 - protein, 11 aa, Artificial Sequence
  hSC27.204 CDRL1
KAGQNVGTSVA

SEQ ID NO: 110 - protein, 7 aa, Artificial Sequence
  hSC27.204 CDRL2
SASYRYS

SEQ ID NO: 111 - protein, 9 aa, Artificial Sequence
  hSC27.204 CDRL3
QQYITYPYT

SEQ ID NO: 112 - protein, 5 aa, Artificial Sequence
  hSC27.204 CDRH1
RYWMS

SEQ ID NO: 113 - protein, 17 aa, Artificial Sequence
  hSC27.204 CDRH2
EINPDSSTINYTPSLKA

SEQ ID NO: 114 - protein, 3 aa, Artificial Sequence
  hSC27.204 CDRH3
PAY

SEQ ID NO: 115 - protein, 17 aa, Artificial Sequence
  hSC27.204v2 CDRH2
EINPDSSTIQYTPSLKA

(19) United States
(12) Patent Application Publication
(10) Pub. No.: US 2019/0083645 A1
(43) Pub. Date: Mar. 21, 2019

(54) NOVEL ANTI-CLAUDIN ANTIBODIES AND METHODS OF USE

(71) Applicant: ABBVIE STEMCENTRX LLC, NORTH CHICAGO, IL (US)

(72) Inventors: SARAH FONG, OAKLAND, CA (US); VIKRAM NATWARSINHJI SISODIYA, SAN FRANCISCO, CA (US); ROBERT A. STULL, ALAMEDA, CA (US); SAMUEL A. WILLIAMS, SAN MATEO, CA (US)

(73) Assignee: ABBVIE STEMCENTRX LLC, NORTH CHICAGO, IL (US)

(21) Appl. No.: 15/781,110

(22) PCT Filed: Dec. 2, 2016
(86) PCT No.: PCT/US2016/064617

§ 371 Date: Jun. 1, 2018

Related U.S. Application Data
(60) Provisional application No. 62/263,542, filed on Dec. 4, 2015.
(60) Provisional application No. 62/427,027, filed on Nov. 28, 2016.

(51) Int. Cl. A61K 47/68; A61P 35/00; A61K 31/551; C07K 16/28

(52) U.S. Cl. CPC A61K 47/6849; A61P 35/00; C07K 16/28; A61K 47/6803; A61K 31/551

(57) ABSTRACT

Provided herein are novel anti-CLDN antibodies and antibody drug conjugates (ADC), including derivatives thereof, and methods of using the same to treat proliferative disorders.

### CROSS REFERENCED APPLICATIONS

This application claims priority to U.S. Provisional Application No. 62/263,542 filed on Dec. 4, 2015 and U.S. Provisional Application No. 62/427,027 filed Nov. 28, 2016 each of which is incorporated herein by reference in its entirety.

### SEQUENCE LISTING

This application contains a sequence listing which has been submitted in ASCII format via EFS-Web and is hereby incorporated by reference in its entirety. Said ASCII copy, created on Dec. 1, 2016, is named sc2704WOO1_S69697_1330WO_SEQL_120116.txt and is 114,404 bytes in size.

### FIELD OF THE INVENTION

This application generally relates to novel anti-claudin (anti-CLDN) antibodies or immunoreactive fragments thereof and compositions, including antibody drug conjugates, comprising the same for the treatment, diagnosis or prophylaxis of cancer and any recurrence or metastasis thereof. Selected embodiments of the invention provide for the use of such anti-CLDN antibodies or antibody drug conjugates for the treatment of cancer comprising a reduction in tumorigenic cell frequency.

### BACKGROUND OF THE INVENTION

Claudins are integral membrane proteins comprising a major structural protein of tight junctions, the most apical cell-cell adhesion junction in polarized cell types such as those found in epithelial or endothelial cell sheets. Tight junctions are composed of strands of networked proteins that form continuous seals around cells to provide a physical but modulatable barrier to the transport of solutes and water in the paracellular space. The claudin family of proteins in humans is comprised of at least 23 members, ranging in size from 22-34 kDa. Although claudins are important in the function and homeostasis of normal tissues, tumor cells frequently exhibit abnormal tight junction function. This may be linked to disregulated expression and/or localization of claudins as a consequence of the dedifferentiation of tumor cells, or the requirement of rapidly growing cancerous tissues to efficiently absorb nutrients within a tumor mass with abnormal vascularization (Morin, 2005, PMID: 16266975). Individual claudin family members may be up-regulated in certain cancer types, yet down-regulated in others. Claudin proteins may be particularly good targets for antibody drug conjugates (ADCs) since it is known that claudins undergo endocytosis, turnover time of some claudins is short relative to other membrane proteins (Van Itallie et al., 2004, PMID: 15366421), claudin expression is disregulated in cancer cells and tight junctions structures among tumor cells are disrupted in cancer cells. These properties may afford more opportunities for antibodies to bind claudin proteins in neoplastic but not in normal tissues. Although antibodies specific to individual claudins may be useful, it is also possible that polyreactive claudin antibody drug conjugates would be more likely to facilitate the delivery of cytotoxins to a broader patient population.

Conventional therapeutic treatments for cancer such as chemotherapy and radiotherapy are often ineffective and surgical resection may not provide a viable clinical alternative. Limitations in the current standard of care are particularly evident in those cases where patients undergo first line treatments and subsequently relapse. In such cases refractory tumors, often aggressive and incurable, frequently arise. There remains therefore a great need to develop more targeted and potent therapies for proliferative disorders. The current invention addresses this need.

### SUMMARY OF THE INVENTION

In a broad aspect the present invention provides isolated antibodies, and corresponding antibody drug or diagnostic conjugates, or compositions thereof, which specifically bind to human CLDN determinants. In certain embodiments the CLDN determinant is a CLDN protein expressed on tumor cells while in other embodiments the CLDN determinant is expressed on tumor initiating cells. In other embodiments the antibodies or ADCs of the invention bind to a CLDN protein and compete for binding with an antibody that binds to an epitope on human CLDN protein

Selected aspects of the invention are directed to antibody drug conjugates (ADC) comprising an antibody that specifically binds to one or more of the claudin (CLDN) family of proteins. In certain embodiments the ADCs of the invention comprise the formula M-[L-D]n wherein: M comprises an anti-CLDN antibody; L comprises an optional linker; D comprises a pyrrolobenzodiazepine (PBD) warhead selected from the group consisting of:

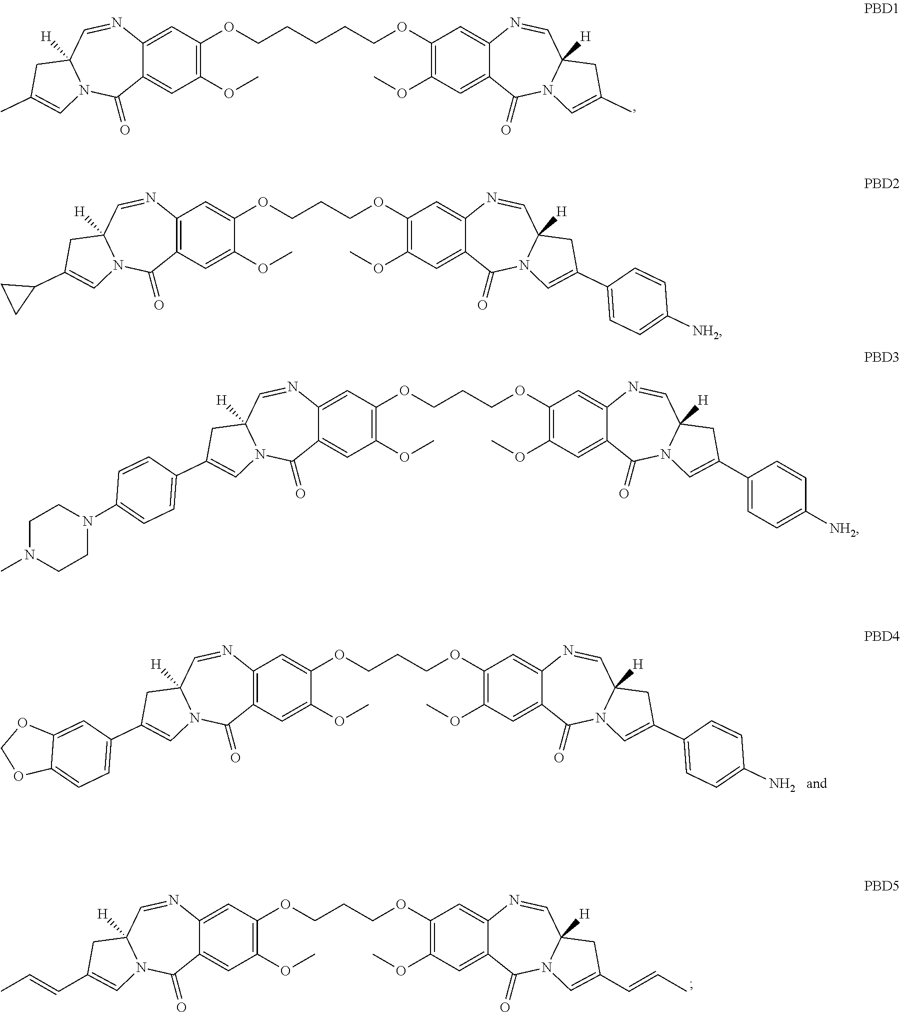

and n comprises and integer from 1 to 20.

In certain aspects the ADCs of the invention comprise an anti-CLDN antibody that is a monoclonal antibody. In a further embodiment the anti-CLDN antibodies comprising the ADCs of the invention are selected from the group consisting of a chimeric antibody, CDR-grafted antibody, humanized antibody, human antibody, primatized antibody, multispecific antibody, bispecific antibody, monovalent antibody, multivalent antibody, anti-idiotypic antibody, diabody, Fab fragment, F(ab′)₂ fragment, Fv fragment, and ScFv fragment; or an immunoreactive fragment thereof. In another embodiment the ADC is comprised of an anti-CLDN antibody that is an internalizing antibody. In a further embodiment the ADCs of the invention bind to cancer stem cells.

In certain aspects the ADCs of the invention comprise an anti-CLDN antibody that comprises or competes for binding to a human CLDN protein with an antibody comprising a light chain variable region (VL) set forth as SEQ ID NO: 21 and a heavy chain variable region (VH) set forth as SEQ ID NO: 23 (SC27.1); or a VL set forth as SEQ ID NO: 25 and a VH set forth as SEQ ID NO: 27 (SC27.22); or a VL set forth as SEQ ID NO: 29 and a VH set forth as SEQ ID NO: 31 (SC27.103); or a VL set forth as SEQ ID NO: 33 and a VH set forth as SEQ ID NO: 35 (SC27.104); or a VL set forth as SEQ ID NO: 37 and a VH set forth as SEQ ID NO: 39 (SC27.105); or a VL set forth as SEQ ID NO: 41 and a VH set forth as SEQ ID NO: 43 (SC27.106); or a VL set forth as SEQ ID NO: 45 and a VH set forth as SEQ ID NO: 47 (SC27.108); or a VL set forth as SEQ ID NO: 49 and a VH set forth as SEQ ID NO: 51 (SC27.201); or a VL set forth as SEQ ID NO: 53 and a VH set forth as SEQ ID NO: 55 (SC27.203); or a VL set forth as SEQ ID NO: 57 and a VH set forth as SEQ ID NO: 59 (SC27.204).

In further aspects ADCs of the invention comprise an anti-CLDN antibody that comprises or competes for binding to a human CLDN protein with an antibody comprising a light chain variable region (VL) set forth as SEQ ID NO: 61 and a heavy chain variable region (VH) set forth as SEQ ID NO: 63 (hSC27.1); or a VL set forth as SEQ ID NO: 65 and a VH set forth as SEQ ID NO: 67 (hSC27.22); or a VL set forth as SEQ ID NO: 69 and a VH set forth as SEQ ID NO: 71 (hSC27.108); or a VL set forth as SEQ ID NO: 73 and a VH set forth as SEQ ID NO: 75 (hSC27.204); or a VL set forth as SEQ ID NO: 73 and a VH set forth as SEQ ID NO: 77 (hSC27.204v2).

In some embodiments the ADCs of the invention comprise an anti-CLDN antibody that comprises or competes for binding to a human CLDN protein with an antibody that comprises a VL having three complimentary determining regions (CDRL): CDRL1 having SEQ ID NO: 109, CDRL2 having SEQ ID NO: 110 and CDRL3 having SEQ ID NO: 111, and a VH having three complimentary determining regions (CDRH): CDRH1 having SEQ ID NO: 112, CDRH2 having SEQ ID NO: 115 and CDRH3 having SEQ ID NO: 114 (hSC27.204v2).

In other embodiments the ADC of the invention comprises an anti-CLDN antibody that comprises or competes for binding to a human CLDN protein with an antibody that comprises a light chain having SEQ ID NO: 78 and a heavy chain having SEQ ID NO: 79 (hSC27.1); or an antibody that comprises a light chain having SEQ ID NO: 80 and a heavy chain having SEQ ID NO: 81 (hSC27.22); or an antibody that comprises a light chain having SEQ ID NO: 80 and a heavy chain having SEQ ID NO: 82 (hSC27.22ss1); or an antibody that comprises a light chain having SEQ ID NO: 83 and a heavy chain having SEQ ID NO: 84 (hSC27.108) or an antibody that comprises a light chain having SEQ ID NO: 83 and a heavy chain having SEQ ID NO: 85 (hSC27.108ss1) or an antibody that comprises a light chain having SEQ ID NO: 86 and a heavy chain having SEQ ID NO: 87 (hSC27.204); or an antibody that comprises a light chain having SEQ ID NO: 86 and a heavy chain having SEQ ID NO: 88 (hSC27.204v2); or an antibody that comprises a light chain having SEQ ID NO: 86 and a heavy chain having SEQ ID NO: 89 (hSC27.204v2ss1).

Certain embodiments of the invention comprise a pharmaceutical composition comprising an ADC as disclosed herein. Other embodiments of the invention comprise a method of treating cancer, for example, ovarian cancer (e.g. ovarian serous carcinoma or ovarian endometrioid adenocarcinoma) or lung cancer (e.g. lung squamous cell carcinoma) or endometrial cancer (e.g. uterine corpus endometrial carcinoma) comprising administering a pharmaceutical composition comprising any of the ADCs of the invention to a subject in need thereof. Another embodiment of the invention is a method of treating cancer with one of the ADCs of the invention and at least one additional therapeutic moiety.

In a further aspect the invention comprises a method of reducing cancer stem cells in a tumor cell population, wherein the method comprises contacting a tumor cell population comprising cancer stem cells and tumor cells other than cancer stem cells, with an anti-CLDN ADC of the invention; whereby the frequency of cancer stem cells is reduced.

In a further embodiment the invention comprise a method of delivering a cytotoxin to a cell comprising contacting the cell with any of the ADCs of the invention.

In a similar vein the present invention also provides kits or devices and associated methods that are useful in the diagnosis, monitoring or treatment of CLDN associated disorders such as cancer. To this end the present invention preferably provides an article of manufacture useful for detecting, diagnosing or treating CLDN associated disorders comprising a receptacle containing a CLDN ADC and instructional materials for using said CLDN ADC to treat, monitor or diagnose the CLDN associated disorder or provide a dosing regimen for the same. In selected embodiments the devices and associated methods will comprise the step of contacting at least one circulating tumor cell. In other embodiments the disclosed kits will comprise instructions, labels, inserts, readers or the like indicating that the kit or device is used for the diagnosis, monitoring or treatment of a CLDN associated cancer or provide a dosing regimen for the same.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the detailed description.

### BRIEF DESCRIPTION OF THE FIGURES

FIGS. 1A and 1B show the sequence relationships between the CLDN proteins. FIG. 1A is a dendrogram generated using an alignment algorithm and the protein sequences derived from the 23 human CLDN genes, showing the close sequence relationship between CLDN6 and CLDN9; FIG. 1B is an amino acid sequence alignment of the CLDN6 protein with the CLDN9 protein, showing identically conserved residues (vertical hash) and an overlay of topological domains (cytoplasmic residues, lower case; transmembrane helices, boxed and upper case; extracellular residues, bold upper case).

FIGS. 2A-2H provide amino acid and nucleic acid sequences of mouse and humanized anti-CLDN antibodies. FIGS. 2A and 2B show light chain (FIG. 2A) and heavy chain (FIG. 2B) variable region amino acid sequences of exemplary mouse and humanized anti-CLDN antibodies and a variant of hSC27.204 (SEQ ID NOS: 21-77, odd numbers). FIG. 2C shows the nucleic acid sequences of the same light and heavy chain variable regions of such exemplary mouse and humanized anti-CLDN antibodies and a variant of hSC27.204 (SEQ ID NOS: 20-76, even numbers). FIG. 2D shows the amino acid sequences of the full length light and heavy chains of humanized antibodies hSC27.1, hSC27.22, hSC27.108 and hSC27.204 and variants of hSC27.22, hSC27.108 and hSC27.204 (SEQ ID NOS: 78-89). FIGS. 2E-2H show annotated amino acid sequences of the light and heavy chain variable regions of the anti-CLDN antibodies, SC27.1 (FIG. 2E), SC27.22 (FIG. 2F), SC27.108 (FIG. 2G), and SC27.204 (FIG. 2H), wherein the CDRs are set forth using Kabat, Chothia, ABM and Contact methodology.

FIG. 3A shows the ability of anti-CLDN antibodies SC27.1 and SC27.22 to bind HEK293T cells overexpressing human CLDN4, CLDN6 and CLDN9 as detected by flow cytometry, where results are shown as change in mean fluorescence intensity (ΔMFI) and a histogram, with the solid black line indicating the binding of the indicated antibody to cells overexpressing the indicated CLDN protein compared to fluorescence minus one (FMO) isotype-control (gray-fill).

FIG. 3B shows the ability of anti-CLDN antibodies to bind HEK293T cells overexpressing CLDN4, CLDN6 and CLDN9 as detected by flow cytometry, where the results are shown as mean fluorescence intensity (MFI) for each antibody binding to each cell line;

FIG. 3C shows the apparent binding affinity of an exemplary anti-CLDN antibody for CLDN6 and CLDN9 as determined by a titration of the amount of antibody versus a fixed number of cells expressing the antigen of interest.

FIG. 4A show that anti-CLDN antibodies SC27.1 and SC27.22 are able to internalize into cells overexpressing human CLDN4, CLDN6 and CLDN9 and mediate the delivery of saporin cytotoxin.

FIG. 4B shows the apparent 1050 of various antibodies for CLDN4, CLDN6 and CLDN9.

FIGS. 5A and 5B show the ability of anti-CLDN ADCs to reduce the volume of ovarian and lung tumors in vivo.

FIG. 6A shows expression of CLDN4, CLDN6, and CLDN9 proteins in human CSC (solid black line) compared to non-tumorigenic (dashed line) ovarian, pancreatic and lung tumor cell populations and FMO isotype controls (gray-fill).

FIG. 6B shows the growth of tumors in mice transplanted with CLDN⁺ (closed circles) or CLDN⁻ (open circles) ovarian tumor cells where CLDN⁺ tumor cells exhibit enhanced tumorigenicity compared to CLDN⁻ ovarian tumor cells.

FIG. 7 shows the results of a limiting dilution assay; tumors treated with anti-CLDN ADC, SC27.22PBD1, showed a reduction in tumor initiating cells of approximately 4-fold compared to tumors treated with control ADC IgG1 PBD1.

FIGS. 8A-8D show, respectively, relative mRNA expression of CLDN6 (FIG. 8A) and of CLDN9 (FIG. 8B) across a series of tumors and normal tissue as derived from The Cancer Genome Atlas while FIG. 8C shows the relative mRNA expression of CLDN family members in uterine corpus endometrial carcinoma as subdivided by tumor stage and FIG. 8D shows the relative mRNA expression of CLDN6 versus hormone receptor expression in stage III and stage IV uterine corpus endometrial carcinoma.

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

CLDN expression has surprisingly been found to be a biological marker of a number of tumor types and this association may be exploited in the treatment of such tumors. It has also unexpectedly been found that CLDN expression is associated with tumorigenic cells and, as such, may be effectively exploited to inhibit or eliminate such cells. Tumorigenic cells, which will be described in more detail below, are known to exhibit resistance to many conventional treatments. In contrast to the teachings of the prior art, the disclosed compounds and methods effectively overcome this inherent resistance.

Thus, it is particularly remarkable that CLDN conjugates such as those disclosed herein may advantageously be used in the treatment and/or prevention of selected proliferative (e.g., neoplastic) disorders or progression or recurrence thereof. It will be appreciated that, while preferred embodiments of the invention will be discussed extensively below, particularly in terms of particular domains, regions or epitopes or in the context of cancer stem cells or tumors comprising neuroendocrine features and their interactions with the disclosed antibody drug conjugates, those skilled in the art will appreciate that the scope of the instant invention is not limited by such exemplary embodiments. Rather, the most expansive embodiments of the present invention and the appended claims are broadly and expressly directed to anti-CLDN antibodies and conjugates, including those disclosed herein, and their use in the treatment and/or prevention of a variety of CLDN associated or mediated disorders, including neoplastic or cell proliferative disorders, regardless of any particular mechanism of action or specifically targeted tumor, cellular or molecular component.

### I. CLAUDIN (CLDN) PHYSIOLOGY

Claudins are integral membrane proteins comprising a major structural protein of tight junctions, the most apical cell-cell adhesion junction in polarized cell types such as those found in epithelial or endothelial cell sheets. Tight junctions are composed of strands of networked proteins that form continuous seals around cells to provide a physical but modulatable barrier to the transport of solutes and water in the paracellular space. The claudin family of proteins in humans is comprised of at least 23 members, ranging in size from 22-34 kDa. All claudins possess a tetraspanin topology in which both protein termini are located on the intracellular face of the membrane, resulting in the formation of two extracellular (EC) loops, EC1 and EC2. The EC loops mediate head-to-head homophilic, and for certain combinations of claudins, heterophilic interactions that lead to formation of tight junctions. The specific claudin-claudin interactions and claudin EC sequences are a key determinant of ion selectivity and tight junction strength (for example, see Nakano et al., 2009, PMID: 19696885). Typically, EC1 is about 50-60 amino acids in size, contains a conserved disulfide bond within a larger W-X(17-22)-W-X(2)-C-X(8-10)-C motif, and numerous charged residues that participate in ion channel formation (Turksen and Troy, 2004, PMID: 15159449). EC2 is smaller than EC1, being approximately 25 amino acids. Due to its helix-turn-helix conformation, it has been suggested that EC2 contributes to dimer or multimer formation of claudins on opposing cell membranes, although mutations in both loops may perturb complex formation. Claudin-claudin complexes in vitro may range in size from dimers to hexamers, depending upon the specific claudins involved (Krause et al., 2008, PMID: 18036336). Individual claudins show a range of tissue specific expression patterns, as well as developmentally regulated expression as determined by PCR analyses (Krause et al., 2008, PMID:18036336; Turksen, 2011, PMID:21526417).

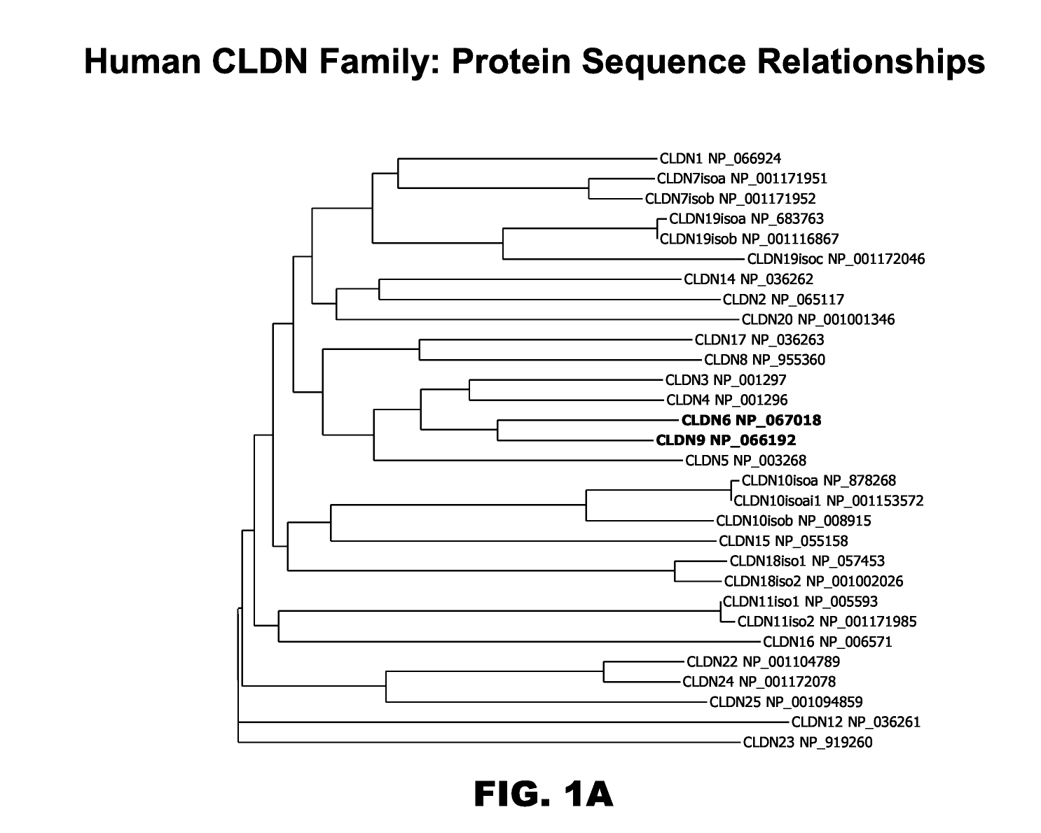

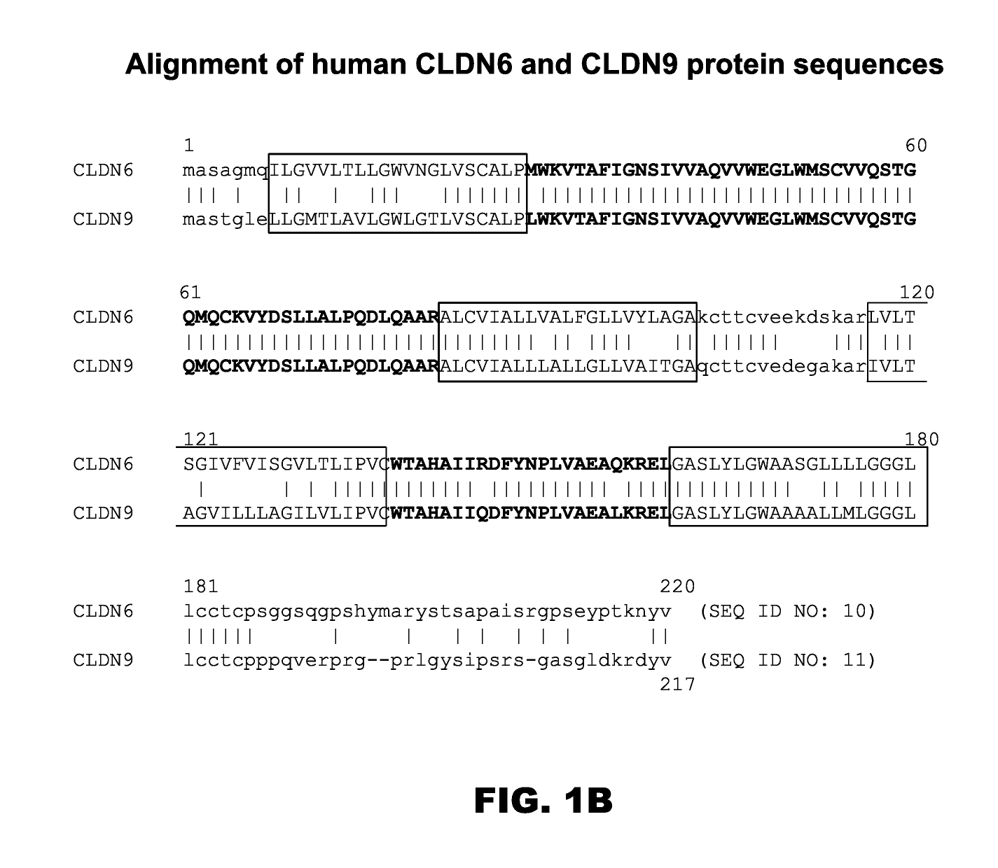

Sequence analysis can be used to construct phylogenetic trees for the claudin family members, indicating the relationship and degrees of relatedness of the protein sequences (FIG. 1A). For instance, it can be seen that the CLDN6 and CLDN9 proteins are closely related which, given the adjacent head-to-head location of their genes at the chromosomal location 16p3.3, is suggestive of an ancestral gene duplication. These similarities likely translate to an ability of these family members to interact heterotypically. Similarly, the CLDN3 and CLDN4 proteins are closely related by sequence analysis, and their genes can be found in tandem at the chromosomal location 7r11.23. High homology in the EC1 or EC2 loops between certain family members (e.g. FIG. 1B) provides opportunity to develop antibodies that are multi-reactive with various claudin family members.

CLDN6, also known as skullin, is a developmentally regulated claudin. Representative CLDN6 protein orthologs include, but are not limited to, human (NP_067018), chimpanzee (XP_523276), rhesus monkey (NP_001180762), mouse (NP_061247), and rat (NP_001095834). In humans, the CLDN6 gene consists of 2 exons spanning approximately 3.5 kBp at the chromosomal location 16p13.3. Transcription of the CLDN6 locus yields a mature 1.4 kB mRNA transcript (NM_021195), encoding a 219 amino acid protein (NP_061247). CLDN6 is expressed in ES cell derivatives committed to an epithelial fate (Turksen and Troy, 2001, PMID: 11668606), in the periderm (Morita et al., 2002, PMID: 12060405), and in the suprabasal level of the epidermis (Turkson and Troy, 2002, PMID: 11923212). It is also expressed in developing mouse kidney (Abuazza et al., 2006, PMID: 16774906), although expression is not detected in adult kidney (Reyes et al., 2002, PMID: 12110008). CLDN6 is also a coreceptor for hepatitis C virus, along with CLDN1 and CLDN9 (Zheng et al., 2007, PMID: 17804490).

CLDN9 is the most closely related family member to CLDN6. Representative CLDN9 protein orthologs include, but are not limited to, human (NP_066192), chimpanzee (XP_003314989), rhesus monkey (NP_001180758), mouse (NP_064689), and rat (NP_001011889). In humans, the CLDN9 gene consists of a single exon spanning approximately 2.1 kBp at the chromosomal locus 16p13.3. Transcription of the intronless CLDN9 locus yields a 2.1 kB mRNA transcript (NM_020982), encoding a 217 amino acid protein (NP_0066192). CLDN9 is expressed in various structures of the inner ear (Kitarjiri et al., 2004, PMID:14698084; Nankano et al., 2009, PMID: 19696885), the cornea (Ban et al., 2003, PMID:12742348), the liver (Zheng et al., 2007, PMID:17804490) and developing kidney (Abuazza et al., 2006, PMID:16774906). Consistent with its expression in the cochlea, animals expressing a CLDN9 protein with a missense mutation show defects in hearing likely due to altered paracellular K⁺ permeability with consequent perturbation of ion currents critical for depolarization of hair cells involved in sound detection. Expression of CLDN9 in cells of the inner ear is specifically localized to a subdomain underneath more apical tight-junction strands formed by other claudins, indicating that not all claudins in normal tissues are found in the most apical and accessible tight junctions (Nankano et al., 2009, PMID: 19696885). In contrast to the results in the cochlea, mice expressing missense CLDN9 showed no signs of hepatic or renal defects (Nankano et al., 2009, PMID: 19696885).

CLDN4 is also known as the Clostridium perfringens enterotoxin receptor, due to its high affinity binding of this toxin responsible for food poisoning and other gastrointestinal illnesses. Representative CLDN4 protein orthologs include, but are not limited to, human (NP_001296), chimpanzee (XP_519142), rhesus monkey (NP_001181493), mouse (NP_034033), and rat (NP_001012022). In humans, the intronless CLDN4 gene spans approximately 1.82 kBp at the chromosomal location 17q11.23. Transcription of the CLDN4 locus yields a 1.82 kB mRNA transcript (NM_001305), encoding a 209 amino acid protein (NP_001296). Consistent with the ability of CLDN4 to bind a toxin produced by a gastrointestinal pathogen, CDLN4 expression can be detected throughout the GI tract as well as in prostate, bladder, breast, and lung (Rahner et al., 2001, PMID:11159882; Tamagawa et al., 2003, PMID:12861044; Wang et al., 2003, PMID:12600828; Nichols et al., 2004, PMID:14983936).

Although claudins are important in the function and homeostasis of normal tissues, tumor cells frequently exhibit abnormal tight junction function. This may be linked to disregulated expression and/or localization of claudins as a consequence of the dedifferentiation of tumor cells, or the requirement of rapidly growing cancerous tissues to efficiently absorb nutrients within a tumor mass with abnormal vascularization (Morin, 2005, PMID: 16266975). Individual claudin family members may be up-regulated in certain cancer types, yet down-regulated in others. For example, CLDN3 and CLDN4 expression is elevated in certain pancreatic, breast and ovarian cancers, yet may be lower in other breast (e.g., “claudin-low”) carcinomas. Claudin proteins may be particularly good targets for antibody drug conjugates (ADCs) since it is known that claudins undergo endocytosis, turnover time of some claudins is short relative to other membrane proteins (Van Itallie et al., 2004, PMID: 15366421), claudin expression is disregulated in cancer cells and tight junctions structures among tumor cells are disrupted in cancer cells. These properties may afford more opportunities for antibodies to bind claudin proteins in neoplastic but not in normal tissues. Although antibodies specific to individual claudins may be useful, it is also possible that polyreactive claudin antibodies would be more likely to facilitate the delivery of payloads to a broader patient population. Specifically, polyreactive claudin antibodies may permit more efficient targeting of cells expressing multiple claudin proteins due to higher aggregate antigen density, reduce the likelihood of escape of tumor cells with low levels of antigen expression of any individual claudin, and as can be seen in the expression examples below, expand the number of therapeutic indications for a single ADC.

### II. CANCER STEM CELLS

According to current models, a tumor comprises non-tumorigenic cells and tumorigenic cells. Non-tumorigenic cells do not have the capacity to self-renew and are incapable of reproducibly forming tumors, even when transplanted into immunocompromised mice in excess cell numbers. Tumorigenic cells, also referred to herein as “tumor initiating cells” (TICs), which typically make up a fraction of the tumor's cell population of 0.01-10%, have the ability to form tumors. For hematopoietic malignancies TICs can be very rare ranging from 1:10⁴ to 1:10⁷ in particular in Acute Myeloid Malignancies (AML) or very abundant for example in lymphoma of the B cell lineage. Tumorigenic cells encompass both tumor perpetuating cells (TPCs), referred to interchangeably as cancer stem cells (CSCs), and tumor progenitor cells (TProgs).

CSCs, like normal stem cells that support cellular hierarchies in normal tissue, are able to self-replicate indefinitely while maintaining the capacity for multilineage differentiation. In this regard CSCs are able to generate both tumorigenic progeny and non-tumorigenic progeny and are able to completely recapitulate the heterogeneous cellular composition of the parental tumor as demonstrated by serial isolation and transplantation of low numbers of isolated CSCs into immunocompromised mice. Evidence indicates that unless these “seed cells” are eliminated tumors are much more likely to metastasize or reoccur leading to relapse and ultimate progression of the disease.

TProgs, like CSCs have the ability to fuel tumor growth in a primary transplant. However, unlike CSCs, they are not able to recapitulate the cellular heterogeneity of the parental tumor and are less efficient at reinitiating tumorigenesis in subsequent transplants because TProgs are typically only capable of a finite number of cell divisions as demonstrated by serial transplantation of low numbers of highly purified TProg into immunocompromised mice. TProgs may further be divided into early TProgs and late TProgs, which may be distinguished by phenotype (e.g., cell surface markers) and their different capacities to recapitulate tumor cell architecture. While neither can recapitulate a tumor to the same extent as CSCs, early TProgs have a greater capacity to recapitulate the parental tumor's characteristics than late TProgs. Notwithstanding the foregoing distinctions, it has been shown that some TProg populations can, on rare occasion, gain self-renewal capabilities normally attributed to CSCs and can themselves become CSCs.

CSCs exhibit higher tumorigenicity and are often relatively more quiescent than: (i) TProgs (both early and late TProgs); and (ii) non-tumorigenic cells such as terminally differentiated tumor cells and tumor-infiltrating cells, for example, fibroblasts/stroma, endothelial and hematopoietic cells that may be derived from CSCs and typically comprise the bulk of a tumor. Given that conventional therapies and regimens have, in large part, been designed to debulk tumors and attack rapidly proliferating cells, CSCs are therefore more resistant to conventional therapies and regimens than the faster proliferating TProgs and other bulk tumor cell populations such as non-tumorigenic cells. Other characteristics that may make CSCs relatively chemoresistant to conventional therapies are increased expression of multi-drug resistance transporters, enhanced DNA repair mechanisms and anti-apoptotic gene expression. Such CSC properties have been implicated in the failure of standard treatment regimens to provide a lasting response in patients with advanced stage neoplasia as standard chemotherapy does not effectively target the CSCs that actually fuel continued tumor growth and recurrence.

It has surprisingly been discovered that CLDN expression is associated with various tumorigenic cell subpopulations in a manner which renders them susceptible to treatment as set forth herein. The invention provides anti-CLDN antibodies that may be particularly useful for targeting tumorigenic cells and may be used to silence, sensitize, neutralize, reduce the frequency, block, abrogate, interfere with, decrease, hinder, restrain, control, deplete, moderate, mediate, diminish, reprogram, eliminate, kill or otherwise inhibit (collectively, “inhibit”) tumorigenic cells, thereby facilitating the treatment, management and/or prevention of proliferative disorders (e.g. cancer). Advantageously, the anti-CLDN antibodies of the invention may be selected so they preferably reduce the frequency or tumorigenicity of tumorigenic cells upon administration to a subject regardless of the form of the CLDN determinant (e.g., phenotypic or genotypic). The reduction in tumorigenic cell frequency may occur as a result of (i) inhibition or eradication of tumorigenic cells; (ii) controlling the growth, expansion or recurrence of tumorigenic cells; (iii) interrupting the initiation, propagation, maintenance, or proliferation of tumorigenic cells; or (iv) by otherwise hindering the survival, regeneration and/or metastasis of the tumorigenic cells. In some embodiments, the inhibition of tumorigenic cells may occur as a result of a change in one or more physiological pathways. The change in the pathway, whether by inhibition or elimination of the tumorigenic cells, modification of their potential (for example, by induced differentiation or niche disruption) or otherwise interfering with the ability of tumorigenic cells to influence the tumor environment or other cells, allows for the more effective treatment of CLDN associated disorders by inhibiting tumorigenesis, tumor maintenance and/or metastasis and recurrence. It will further be appreciated that the same characteristics of the disclosed antibodies make them particularly effective at treating recurrent tumors which have proved resistant or refractory to standard treatment regimens.

Methods that can be used to assess the reduction in the frequency of tumorigenic cells, include but are not limited to, cytometric or immunohistochemical analysis, preferably by in vitro or in vivo limiting dilution analysis (Dylla et al. 2008, PMID: PMC2413402 and Hoey et al. 2009, PMID: 19664991).

In vitro limiting dilution analysis may be performed by culturing fractionated or unfractionated tumor cells (e.g. from treated and untreated tumors, respectively) on solid medium that fosters colony formation and counting and characterizing the colonies that grow. Alternatively, the tumor cells can be serially diluted onto plates with wells containing liquid medium and each well can be scored as either positive or negative for colony formation at any time after inoculation but preferably more than 10 days after inoculation.

In vivo limiting dilution is performed by transplanting tumor cells, from either untreated controls or from tumors exposed to selected therapeutic agents, into immunocompromised mice in serial dilutions and subsequently scoring each mouse as either positive or negative for tumor formation. The scoring may occur at any time after the implanted tumors are detectable but is preferably done 60 or more days after the transplant. The analysis of the results of limiting dilution experiments to determine the frequency of tumorigenic cells is preferably done using Poisson distribution statistics or assessing the frequency of predefined definitive events such as the ability to generate tumors in vivo or not (Fazekas et al., 1982, PMID: 7040548).

Flow cytometry and immunohistochemistry may also be used to determine tumorigenic cell frequency. Both techniques employ one or more antibodies or reagents that bind art recognized cell surface proteins or markers known to enrich for tumorigenic cells (see WO 2012/031280). As known in the art, flow cytometry (e.g. florescence activated cell sorting (FACS)) can also be used to characterize, isolate, purify, enrich or sort for various cell populations including tumorigenic cells. Flow cytometry measures tumorigenic cell levels by passing a stream of fluid, in which a mixed population of cells is suspended, through an electronic detection apparatus which is able to measure the physical and/or chemical characteristics of up to thousands of particles per second. Immunohistochemistry provides additional information in that it enables visualization of tumorigenic cells in situ (e.g., in a tissue section) by staining the tissue sample with labeled antibodies or reagents which bind to tumorigenic cell markers.

As such, the antibodies of the invention may be useful for identifying, characterizing, monitoring, isolating, sectioning or enriching populations or subpopulations of tumorigenic cells through methods such as, for example, flow cytometry, magnetic activated cell sorting (MACS), laser mediated sectioning or FACS. FACS is a reliable method used to isolate cell subpopulations at more than 99.5% purity based on specific cell surface markers. Other compatible techniques for the characterization and manipulation of tumorigenic cells including CSCs can be seen, for example, in U.S. patent Ser. Nos. 12/686,359, 12/669,136 and 12/757,649.

Listed below are markers that have been associated with CSC populations and have been used to isolate or characterize CSCs: ABCA1, ABCA3, ABCB5, ABCG2, ADAMS, ADCY9, ADORA2A, ALDH, AFP, AXIN1, B7H3, BCL9, Bmi-1, BMP-4, C20orf52, C4.4A, carboxypeptidase M, CAV1, CAV2, CD105, CD117, CD123, CD133, CD14, CD16, CD166, CD16a, CD16b, CD2, CD20, CD24, CD29, CD3, CD31, CD324, CD325, CD33, CD34, CD38, CD44, CD45, CD46, CD49b, CD49f, CD56, CD64, CD74, CD9, CD90, CD96, CEACAM6, CELSR1, CLEC12A, CPD, CRIM1, CX3CL1, CXCR4, DAF, decorin, easyh1, easyh2, EDG3, EGFR, ENPP1, EPCAM, EPHA1, EPHA2, FLJ10052, FLVCR, FZD1, FZD10, FZD2, FZD3, FZD4, FZD6, FZD7, FZD8, FZD9, GD2, GJA1, GLI1, GL12, GPNMB, GPR54, GPRCSB, HAVCR2, IL1R1, IL1RAP, JAMS, Lgr5, Lgr6, LRP3, LY6E, MCP, mf2, mllt3, MPZL1, MUC1, MUC16, MYC, N33, NANOG, NB84, NES, NID2, NMA, NPC1, OSM, OCT4, OPN3, PCDH7, PCDHA10, PCDHB2, PPAP2C, PTPN3, PTS, RARRES1, SEMA4B, SLC19A2, SLC1A1, SLC39A1, SLC4A11, SLC6A14, SLC7A8, SMARCA3, SMARCD3, SMARCE1, SMARCAS, SOX1, STAT3, STEAP, TCF4, TEM8, TGFBR3, TMEPAI, TMPRSS4, TFRC, TRKA, WNT10B, WNT16, WNT2, WNT2B, WNT3, WNTSA, YY1 and CTNNB1. See, for example, Schulenburg et al., 2010, PMID: 20185329, U.S. Pat. No. 7,632,678 and U.S.P.N.s. 2007/0292414, 2008/0175870, 2010/0275280, 2010/0162416 and 2011/0020221.

Similarly, non-limiting examples of cell surface phenotypes associated with CSCs of certain tumor types include CD44^{hi}CD24^{low}, ALDH⁺, CD133⁺, CD123⁺, CD34⁺CD38⁻, CD44⁺CD24⁻, CD46^{hi}CD324⁺CD66c⁻, CD133⁺CD34⁺CD10⁻CD19⁻, CD138⁻CD34⁻CD19⁺, CD133±RC2⁺, CD44⁺α₂β₁^{hi}CD133⁺, CD44⁺CD24⁺ESA⁺, CD271⁺, ABCB5⁺ as well as other CSC surface phenotypes that are known in the art. See, for example, Schulenburg et al., 2010, supra, Visvader et al., 2008, PMID: 18784658 and U.S.P.N. 2008/0138313. Of particular interest with respect to the instant invention are CSC preparations comprising CD46^{hi}CD324⁺ phenotypes in solid tumors and CD34⁺CD38⁻ in leukemias.

“Positive,” “low” and “negative” expression levels as they apply to markers or marker phenotypes are defined as follows. Cells with negative expression (i.e. “−”) are herein defined as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one”, or “FMO”, staining. Cells with expression greater than the 95th percentile of expression observed with an isotype control antibody using the FMO staining procedure described above are herein defined as “positive” (i.e. “+”). As defined herein there are various populations of cells broadly defined as “positive.” A cell is defined as positive if the mean observed expression of the antigen is above the 95th percentile determined using FMO staining with an isotype control antibody as described above. The positive cells may be termed cells with low expression (i.e. “lo”) if the mean observed expression is above the 95^{th} percentile determined by FMO staining and is within one standard deviation of the 95^{th} percentile. Alternatively, the positive cells may be termed cells with high expression (i.e. “hi”) if the mean observed expression is above the 95^{th} percentile determined by FMO staining and greater than one standard deviation above the 95^{th} percentile. In other embodiments the 99th percentile may preferably be used as a demarcation point between negative and positive FMO staining and in some embodiments the percentile may be greater than 99%.

The CD46^{hi}CD324⁺ or CD34⁺CD38⁻ marker phenotype and those exemplified immediately above may be used in conjunction with standard flow cytometric analysis and cell sorting techniques to characterize, isolate, purify or enrich TIC and/or TPC cells or cell populations for further analysis.

The ability of the antibodies of the current invention to reduce the frequency of tumorigenic cells can therefore be determined using the techniques and markers described above. In some instances, the anti-CLDN antibodies may reduce the frequency of tumorigenic cells by 10%, 15%, 20%, 25%, 30% or even by 35%. In other embodiments, the reduction in frequency of tumorigenic cells may be in the order of 40%, 45%, 50%, 55%, 60% or 65%. In certain embodiments, the disclosed compounds my reduce the frequency of tumorigenic cells by 70%, 75%, 80%, 85%, 90% or even 95%. It will be appreciated that any reduction of the frequency of tumorigenic cells is likely to result in a corresponding reduction in the tumorigenicity, persistence, recurrence and aggressiveness of the neoplasia.

### III. ANTIBODIES

A. Antibody Structure

Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

An “antibody” or “intact antibody” typically refers to a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As used herein the term “antibody” includes polyclonal antibodies, multiclonal antibodies, monoclonal antibodies, chimeric antibodies, humanized and primatized antibodies, CDR grafted antibodies, human antibodies (including recombinantly produced human antibodies), recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). Non-covalent association between the VH and the VL region forms the Fv fragment (for “fragment variable”) which contains one of the two antigen-binding sites of the antibody.

As used herein, the assignment of amino acids to each domain, framework region and CDR may be in accordance with one of the schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. As is well known in the art variable region residue numbering is typically as set forth in Chothia or Kabat. Amino acid residues which comprise CDRs as defined by Kabat, Chothia, MacCallum (also known as Contact) and AbM as obtained from the Abysis website database (infra.) are set out below in Table 1. Note that MacCallum uses the Chothia numbering system.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005).

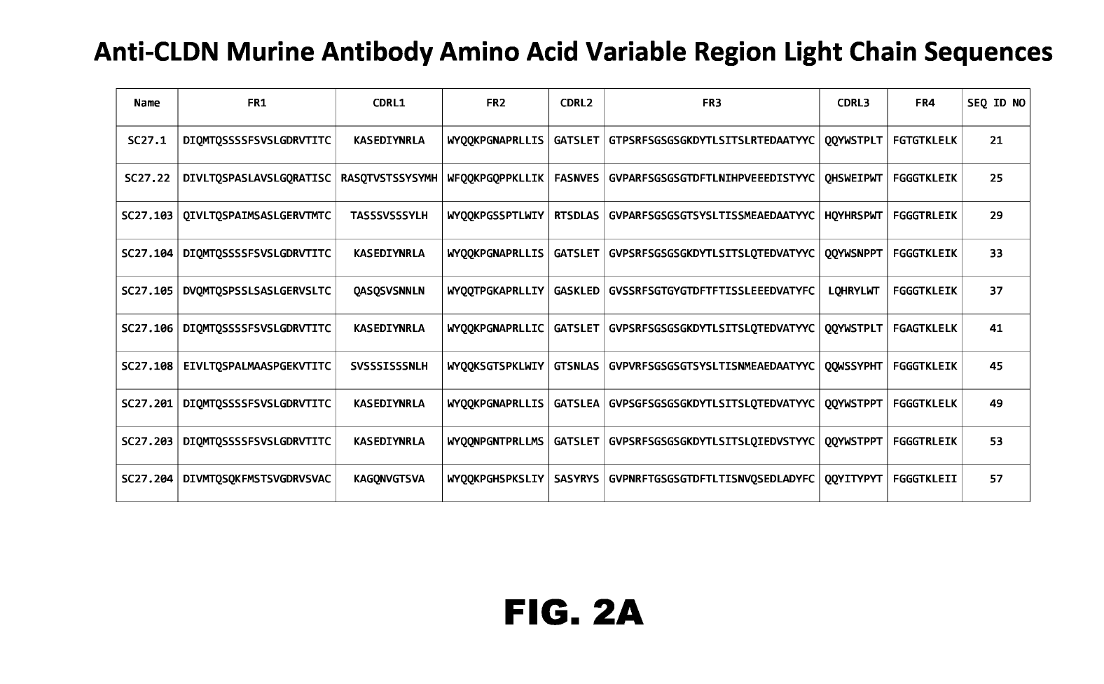

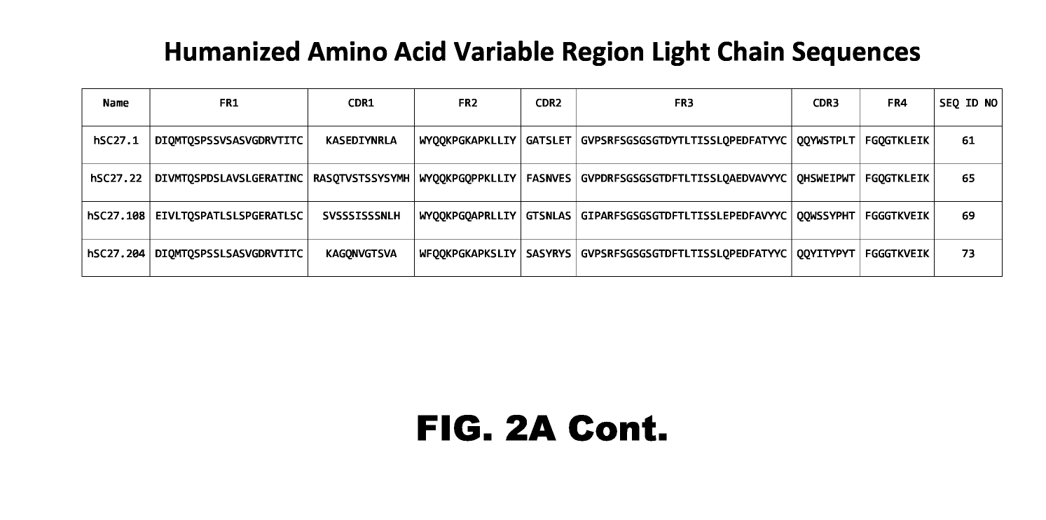

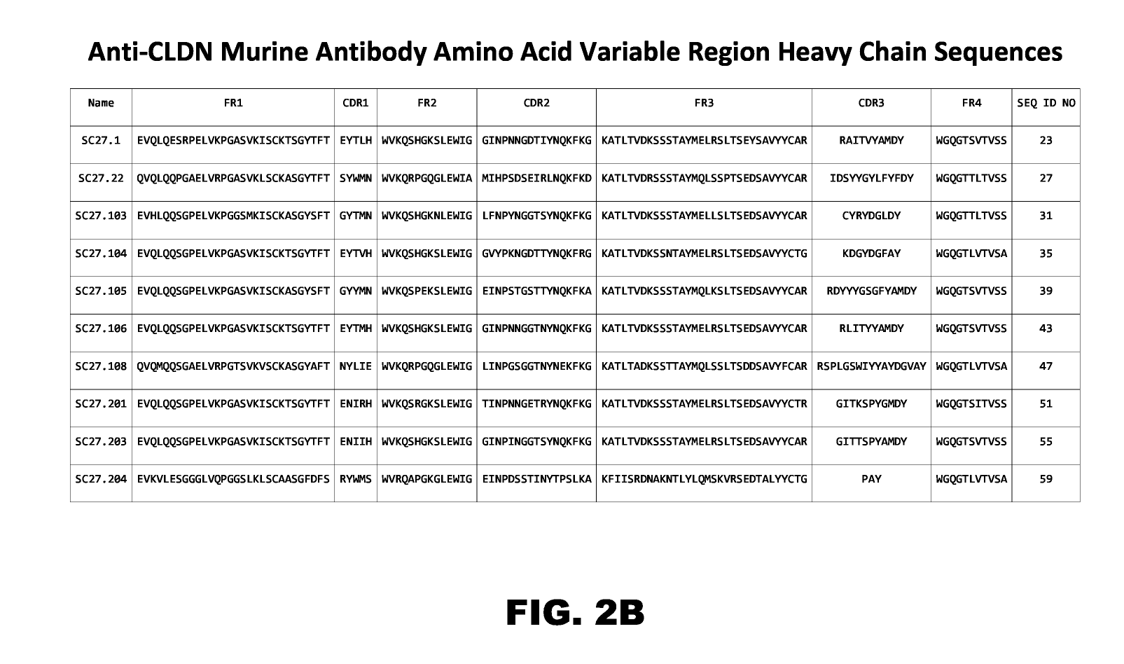

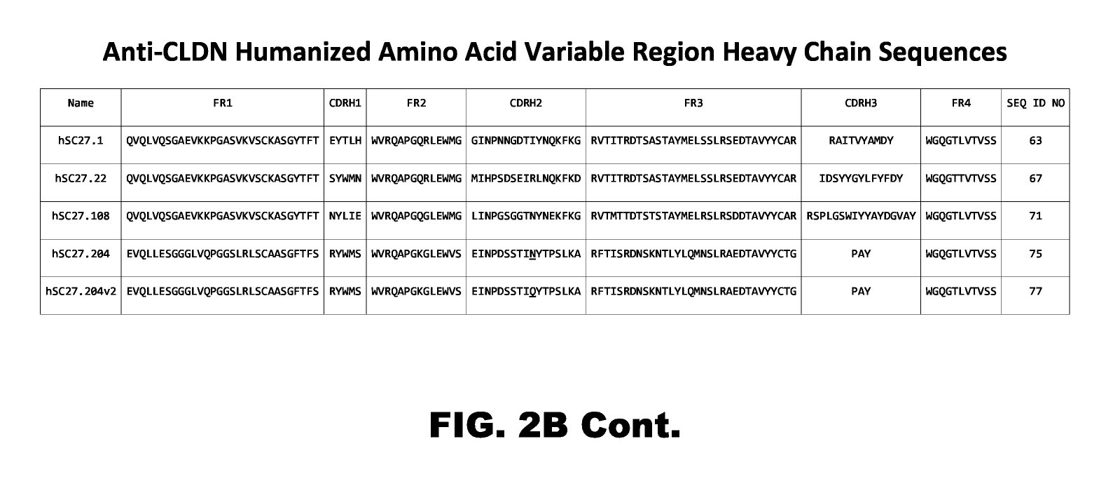

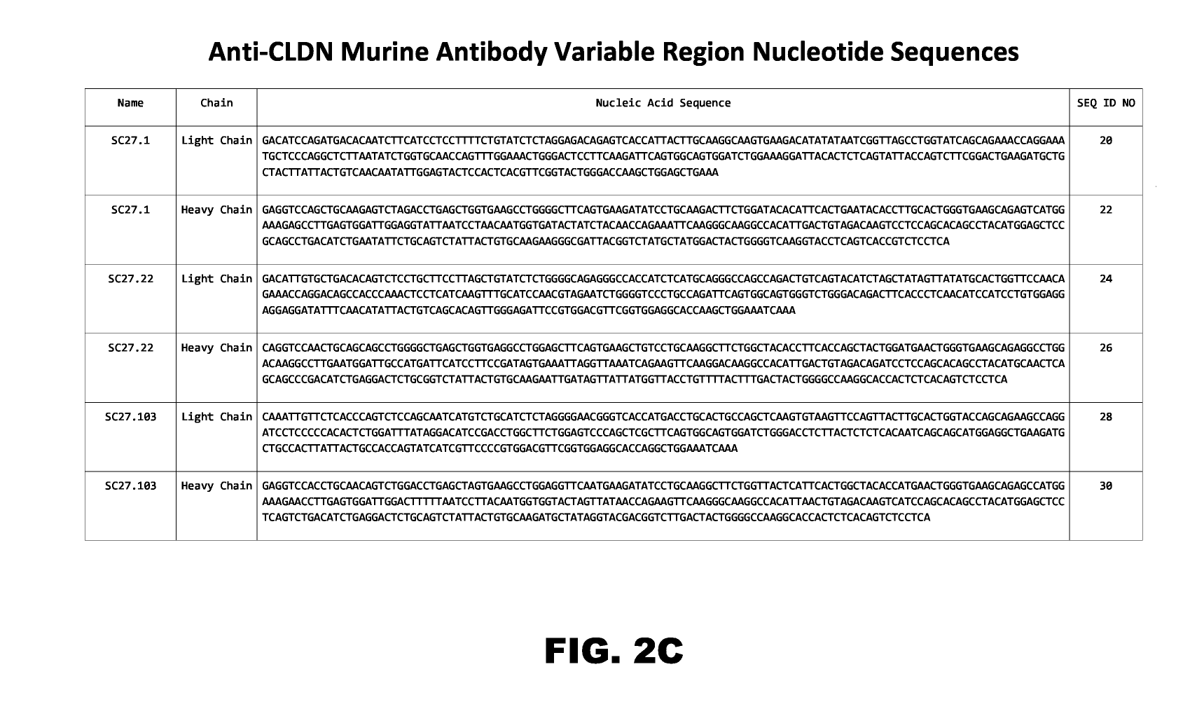

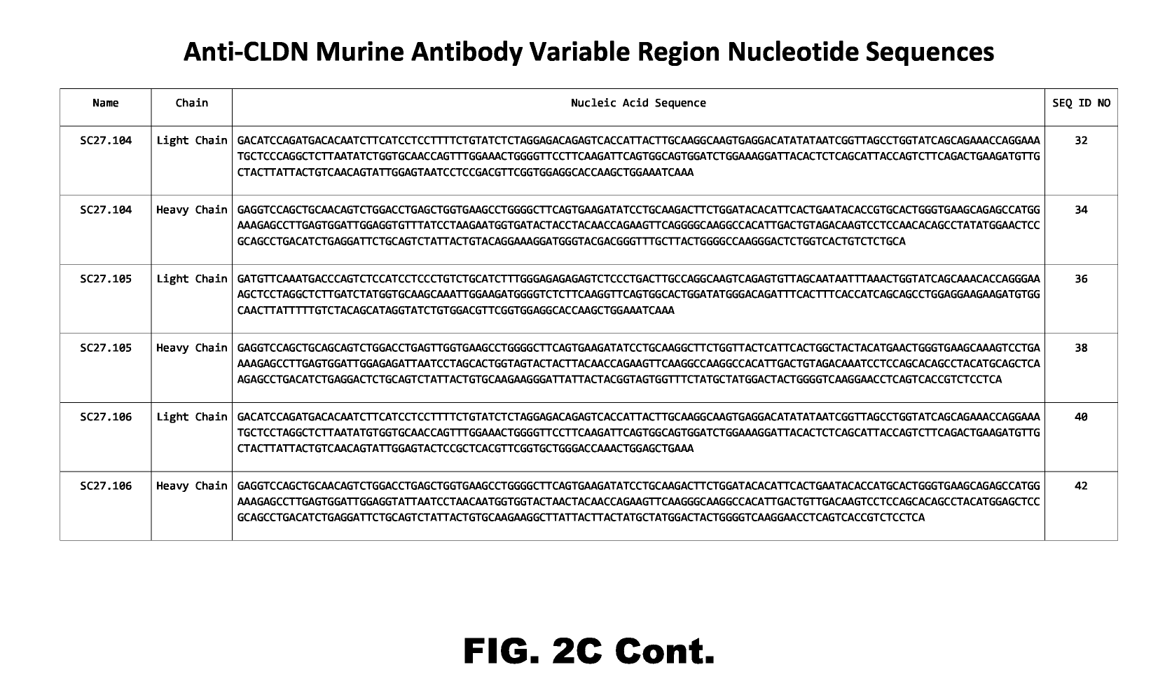

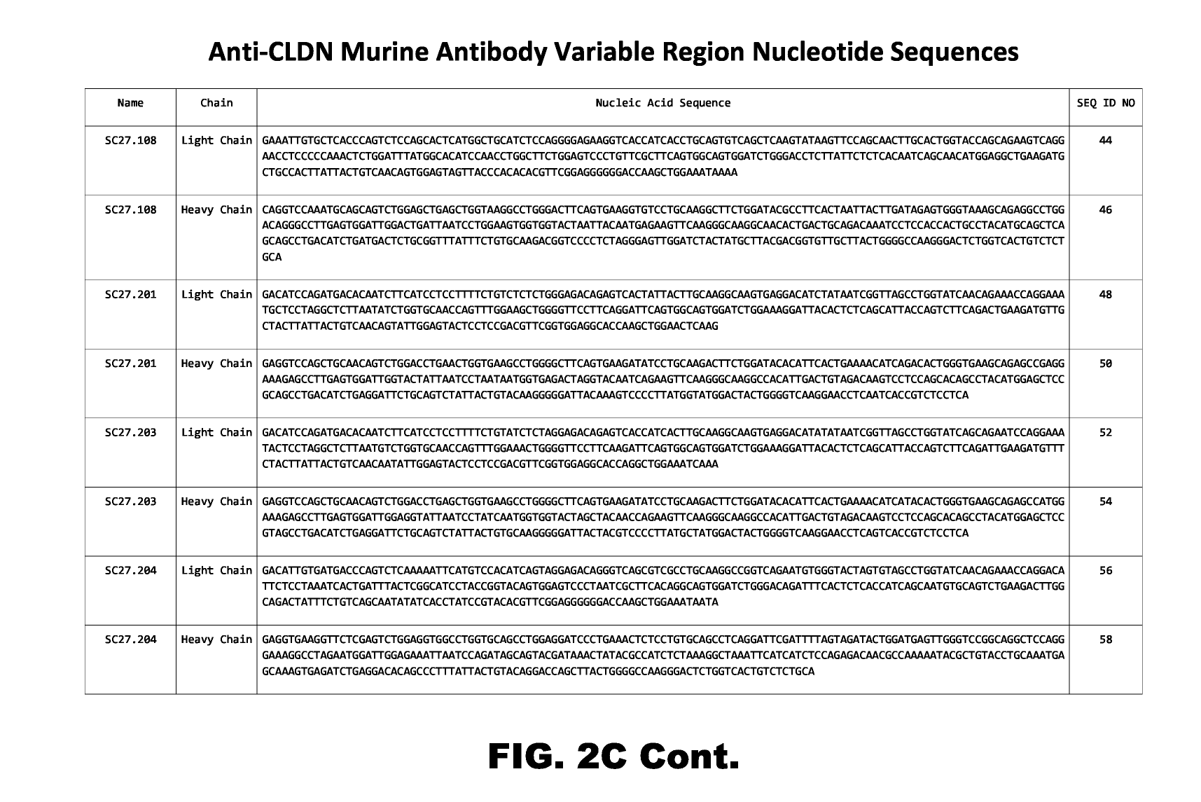

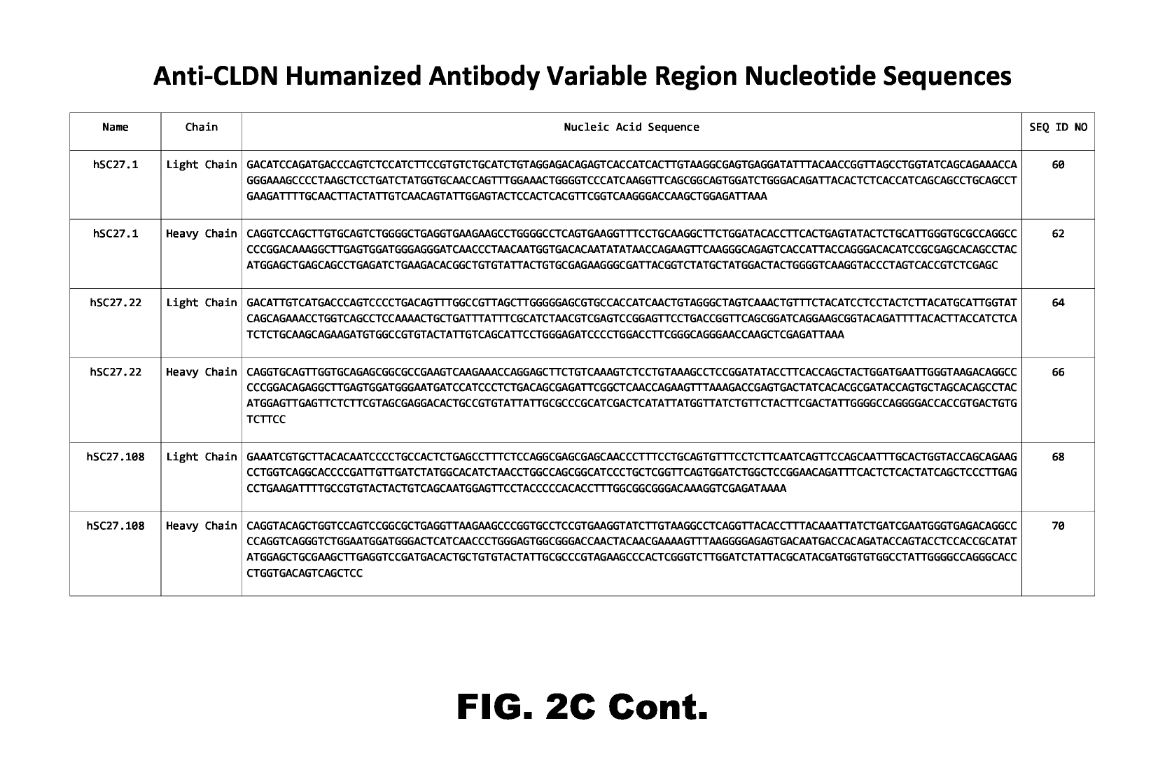

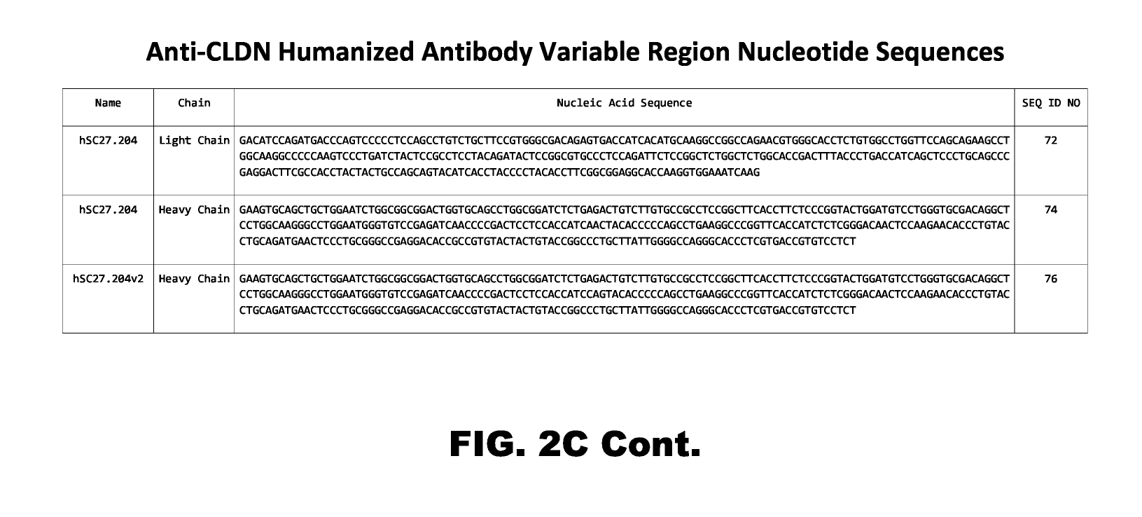

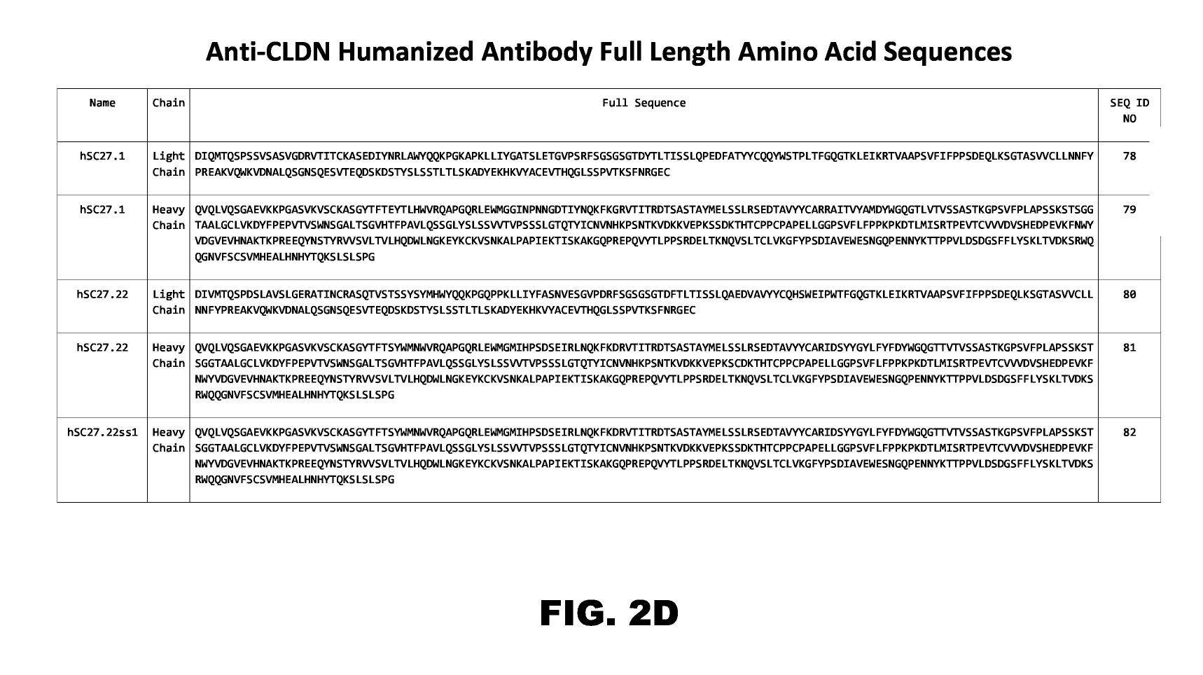

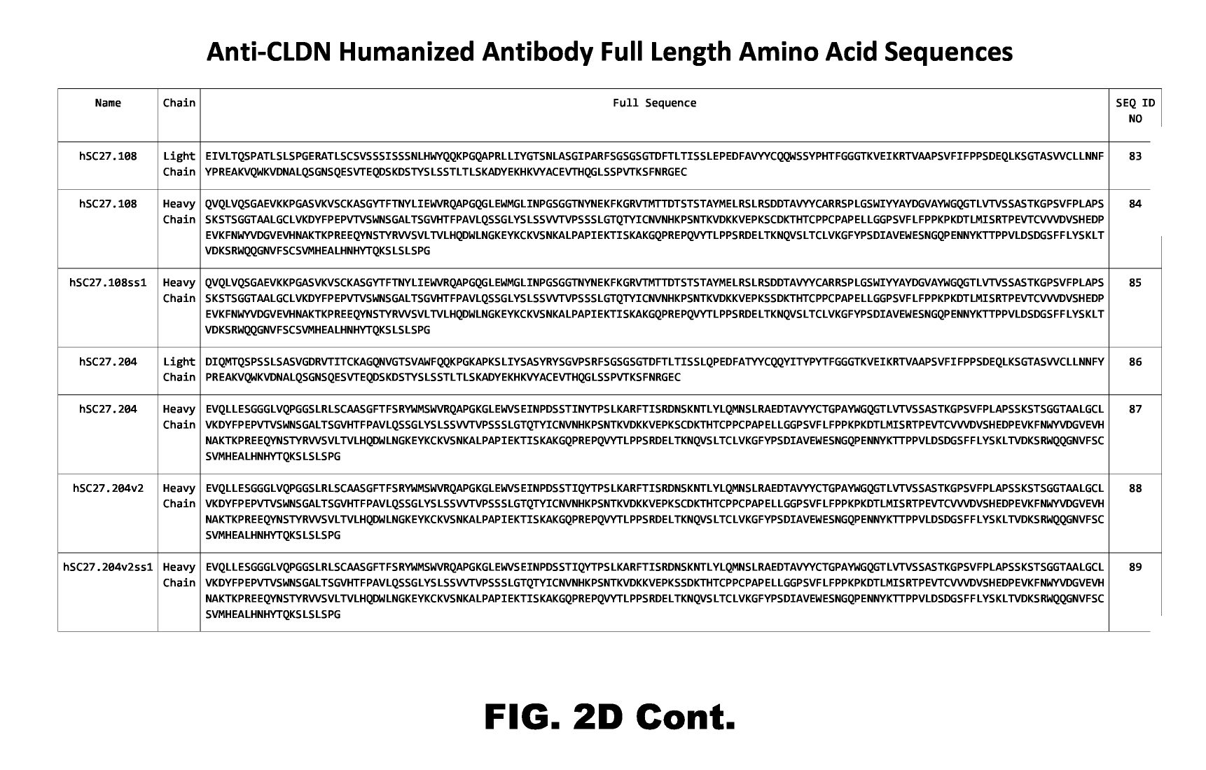

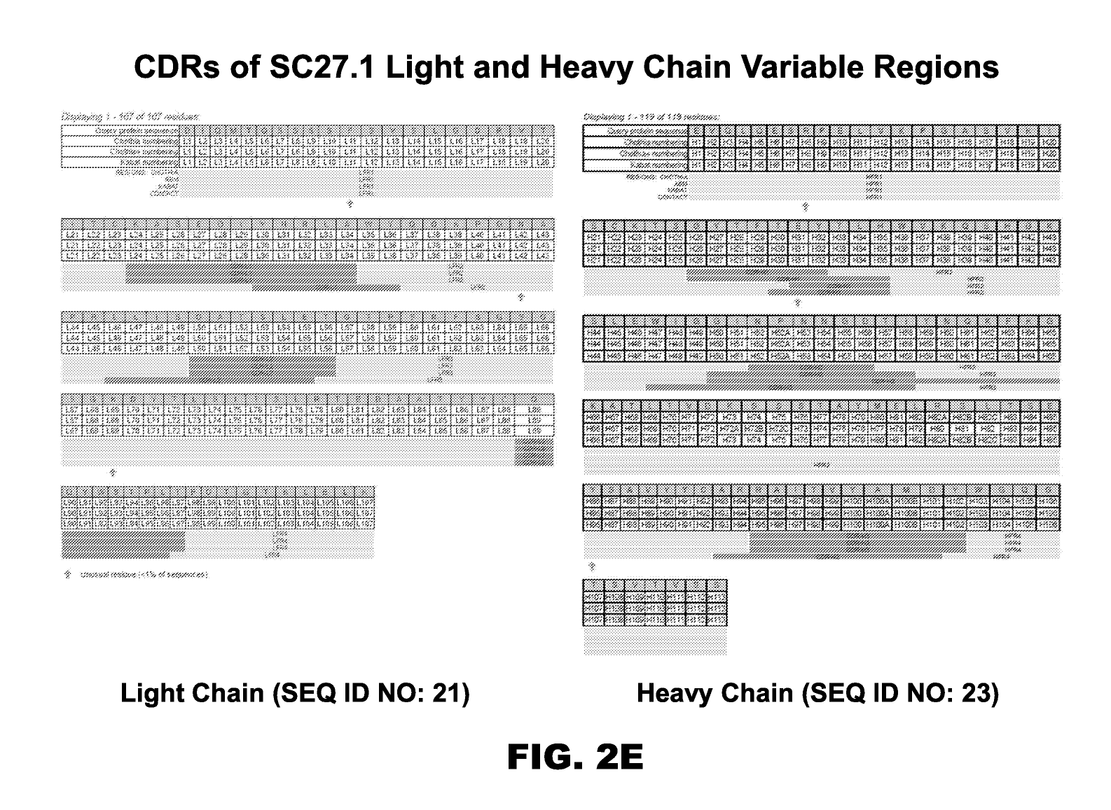

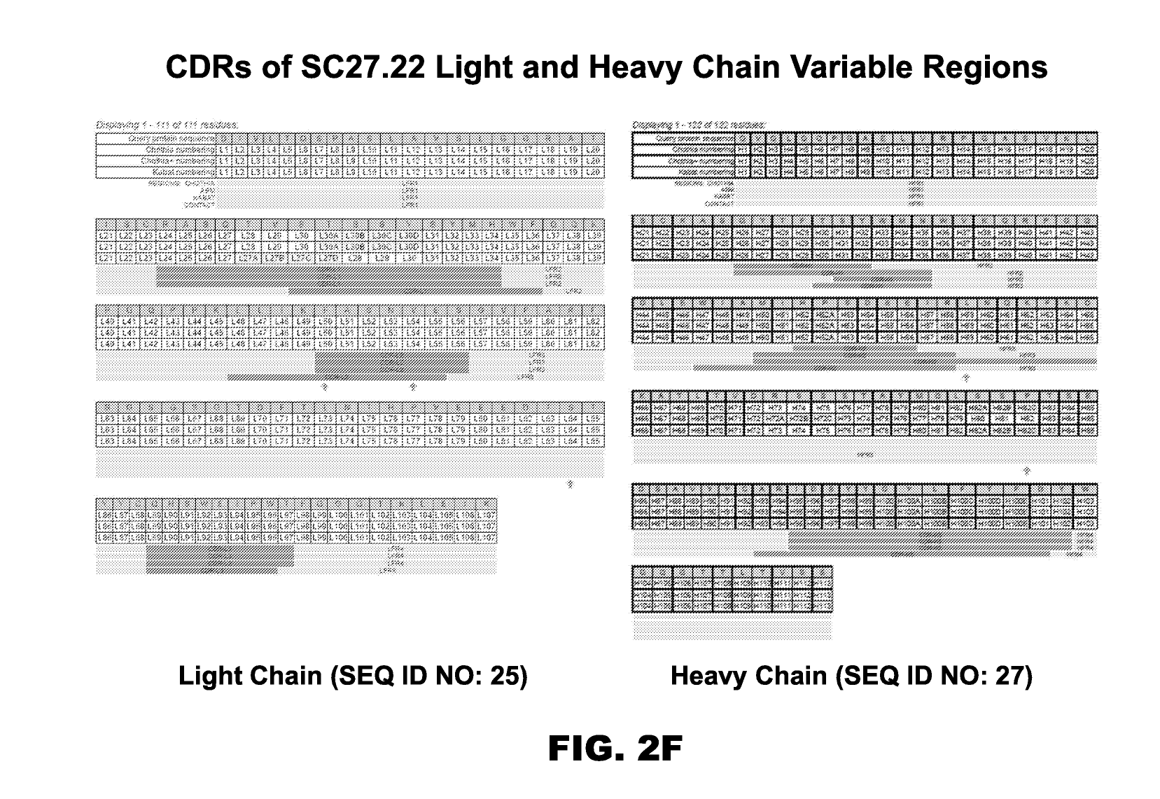

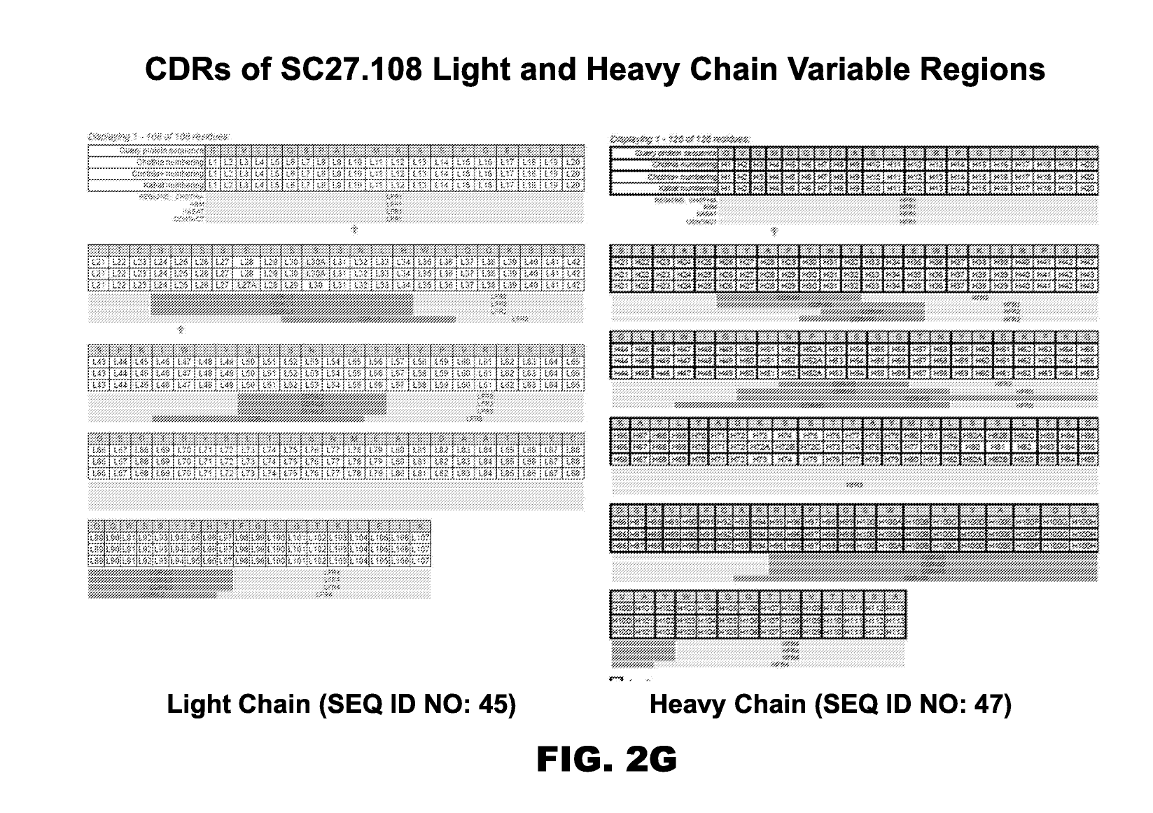

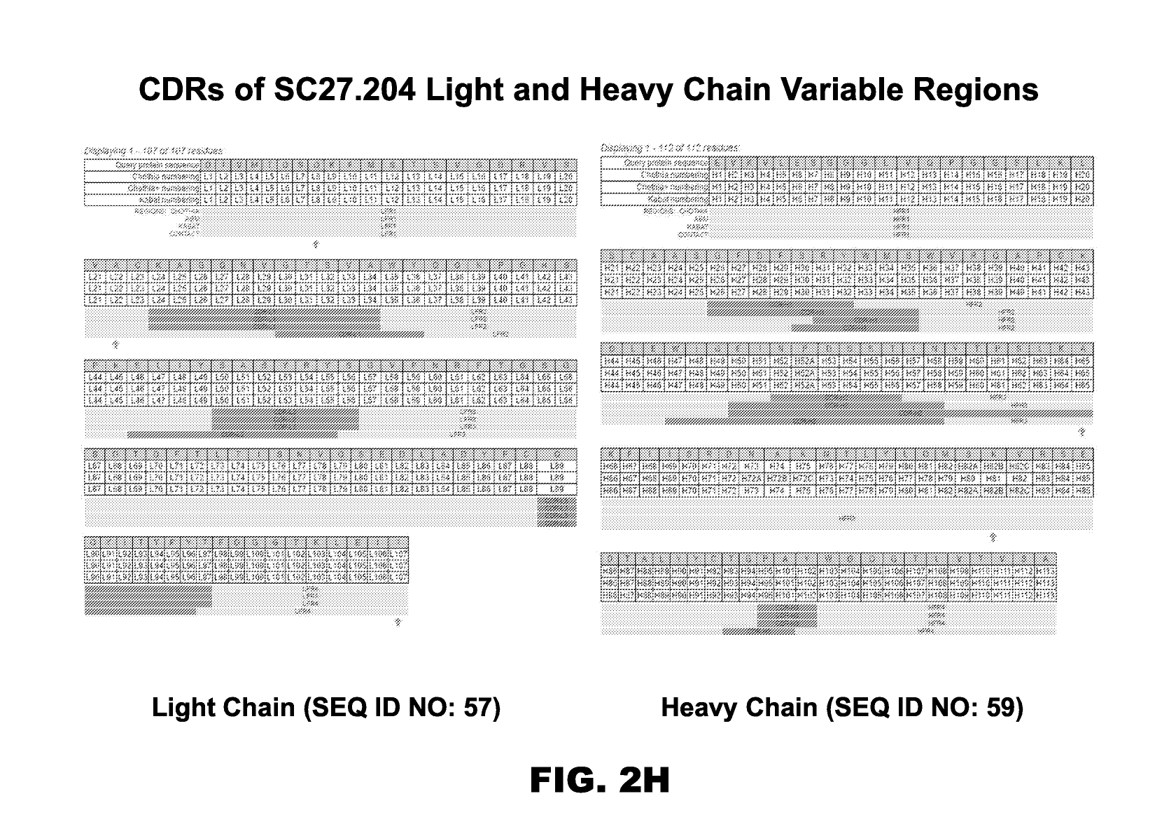

Preferably the sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. FIGS. 2E-2H appended hereto show the results of such analysis in the annotation of exemplary heavy and light chain variable regions for the SC27.1, SC27.22 and SC27.108 and SC27.204 murine antibodies. Unless otherwise indicated, all CDRs set forth herein are derived according to the Abysis database website as per Kabat et al.

For heavy chain constant region amino acid positions discussed in the invention, numbering is according to the Eu index first described in Edelman et al., 1969, Proc. Natl. Acad. Sci. USA 63(1): 78-85 describing the amino acid sequence of the myeloma protein Eu, which reportedly was the first human IgG1 sequenced. The Eu index of Edelman is also set forth in Kabat et al., 1991 (supra.). Thus, the terms “Eu index as set forth in Kabat” or “Eu index of Kabat” or “Eu index” or “Eu numbering” in the context of the heavy chain refers to the residue numbering system based on the human IgG1 Eu antibody of Edelman et al. as set forth in Kabat et al., 1991 (supra.) The numbering system used for the light chain constant region amino acid sequence is similarly set forth in Kabat et al., (supra.) An exemplary kappa light chain constant region amino acid sequence compatible with the present invention is set forth as SEQ ID NO: 4 and an exemplary lambda light chain constant region amino acid sequence compatible with the present invention is set forth as SEQ ID NO: 7. Similarly, an exemplary IgG1 heavy chain constant region amino acid sequence compatible with the present invention is set forth as SEQ ID NO: 1.

The disclosed constant region sequences, or variations or derivatives thereof, may be operably associated with the disclosed heavy and light chain variable regions using standard molecular biology techniques to provide full-length antibodies that may be used as such or incorporated in the anti-CLDN ADCs of the invention.

There are two types of disulfide bridges or bonds in immunoglobulin molecules: interchain and intrachain disulfide bonds. As is well known in the art the location and number of interchain disulfide bonds vary according to the immunoglobulin class and species. While the invention is not limited to any particular class or subclass of antibody, the IgG1 immunoglobulin shall be used throughout the instant disclosure for illustrative purposes. In wild-type IgG1 molecules there are twelve intrachain disulfide bonds (four on each heavy chain and two on each light chain) and four interchain disulfide bonds. Intrachain disulfide bonds are generally somewhat protected and relatively less susceptible to reduction than interchain bonds. Conversely, interchain disulfide bonds are located on the surface of the immunoglobulin, are accessible to solvent and are usually relatively easy to reduce. Two interchain disulfide bonds exist between the heavy chains and one from each heavy chain to its respective light chain. It has been demonstrated that interchain disulfide bonds are not essential for chain association. The IgG1 hinge region contain the cysteines in the heavy chain that form the interchain disulfide bonds, which provide structural support along with the flexibility that facilitates Fab movement. The heavy/heavy IgG1 interchain disulfide bonds are located at residues C226 and C229 (Eu numbering) while the IgG1 interchain disulfide bond between the light and heavy chain of IgG1 (heavy/light) are formed between C214 of the kappa or lambda light chain and C220 in the upper hinge region of the heavy chain.

B. Antibody Generation and Production

Antibodies of the invention can be produced using a variety of methods known in the art.

1. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

In this regard antibodies of the invention may be generated from any CLDN determinant that induces an immune response in an immunocompetent animal. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a CLDN protein, or any of its splice variants, isoforms, homologs or family members, or specific domains, regions or epitopes thereof. An “antigen”, “immunogenic determinant”, “antigenic determinant” or “immunogen” means any CLDN protein or any fragment, region or domain thereof that can stimulate an immune response when introduced into an immunocompetent animal and is recognized by the antibodies produced by the immune response. The presence or absence of the CLDN determinants contemplated herein may be used to identify a cell, cell subpopulation or tissue (e.g., tumors, tumorigenic cells or CSCs).

Any form of antigen, or cells or preparations containing the antigen, can be used to generate an antibody that is specific for the CLDN determinant. As set forth herein the term “antigen” is used in a broad sense and may comprise any immunogenic fragment or determinant of the selected target including a single epitope, multiple epitopes, single or multiple domains or the entire extracellular domain (ECD) or protein. The antigen may be an isolated full-length protein, a cell surface protein (e.g., immunizing with cells expressing at least a portion of the antigen on their surface), or a soluble protein (e.g., immunizing with only the ECD portion of the protein) or protein construct (e.g., Fc-antigen). The antigen may be produced in a genetically modified cell. Any of the aforementioned antigens may be used alone or in combination with one or more immunogenicity enhancing adjuvants known in the art. DNA encoding the antigen may be genomic or non-genomic (e.g., cDNA) and may encode at least a portion of the ECD, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

2. Monoclonal Antibodies

In selected embodiments, the invention contemplates use of monoclonal antibodies. As known in the art, the term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following production of multiple monoclonal antibodies that bind specifically to a determinant, particularly effective antibodies may be selected through various screening processes, based on, for example, its affinity for the determinant or rate of internalization. Antibodies produced as described herein may be used as “source” antibodies and further modified to, for example, improve affinity for the target, improve its production in cell culture, reduce immunogenicity in vivo, create multispecific constructs, etc. A more detailed description of monoclonal antibody production and screening is set out below and in the appended Examples.

3. Human Antibodies

In an antibody” refers to an antibody which possesses an amino acid sequence that corresponds to that of an antibody produced by a human and/or has been made using any of the techniques for making human antibodies described below.

Human antibodies can be produced using various techniques known in the art. One technique is phage display in which a library of (preferably human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; and Barbas et al., Proc. Natl. Acad. Sci. USA 88:7978-7982 (1991)).

In one embodiment, recombinant human antibodies may be isolated by screening a recombinant combinatorial antibody library prepared as above. In one embodiment, the library is a scFv phage display library, generated using human VL and VH cDNAs prepared from mRNA isolated from B-cells.

The antibodies produced by naive libraries (either natural or synthetic) can be of moderate affinity (Kₐ of about 10⁶ to 10⁷ M⁻¹), but affinity maturation can also be mimicked in vitro by constructing and reselecting from secondary libraries as described in the art. For example, mutation can be introduced at random in vitro by using error-prone polymerase (reported in Leung et al., Technique, 1: 11-15 (1989)). Additionally, affinity maturation can be performed by randomly mutating one or more CDRs, e.g. using PCR with primers carrying random sequence spanning the CDR of interest, in selected individual Fv clones and screening for higher-affinity clones. WO 9607754 described a method for inducing mutagenesis in a CDR of an immunoglobulin light chain to create a library of light chain genes. Another effective approach is to recombine the VH or VL domains selected by phage display with repertoires of naturally occurring V domain variants obtained from unimmunized donors and to screen for higher affinity in several rounds of chain reshuffling as described in Marks et al., Biotechnol., 10: 779-783 (1992). This technique allows the production of antibodies and antibody fragments with a dissociation constant K_{D} (k_{off}/kₒₙ) of about 10⁻⁹ M or less.

In other embodiments, similar procedures may be employed using libraries comprising eukaryotic cells (e.g., yeast) that express binding pairs on their surface. See, for example, U.S. Pat. No. 7,700,302 and U.S. Ser. No. 12/404,059. In one embodiment, the human antibody is selected from a phage library, where that phage library expresses human antibodies (Vaughan et al. Nature Biotechnology 14:309-314 (1996): Sheets et al. Proc. Natl. Acad. Sci. USA 95:6157-6162 (1998). In other embodiments, human binding pairs may be isolated from combinatorial antibody libraries generated in eukaryotic cells such as yeast. See e.g., U.S. Pat. No. 7,700,302. Such techniques advantageously allow for the screening of large numbers of candidate modulators and provide for relatively easy manipulation of candidate sequences (e.g., by affinity maturation or recombinant shuffling).

Human antibodies can also be made by introducing human immunoglobulin loci into transgenic animals, e.g., mice in which the endogenous immunoglobulin genes have been partially or completely inactivated and human immunoglobulin genes have been introduced. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and U.S. Pat. Nos. 6,075,181 and 6,150,584 regarding XenoMouse® technology; and Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., J. Immunol, 147 (I):86-95 (1991); and U.S. Pat. No. 5,750,373.

Whatever the source it will be appreciated that the human antibody sequence may be fabricated using art-known molecular engineering techniques and introduced into expression systems and host cells as described herein. Such non-natural recombinantly produced human antibodies (and subject compositions) are entirely compatible with the teachings of this disclosure and are expressly held to be within the scope of the instant invention. In certain select aspects the CLDN ADCs of the invention will comprise a recombinantly produced human antibody acting as a cell binding agent.

4. Derived Antibodies:

Once source antibodies have been generated, selected and isolated as described above they may be further altered to provide anti-CLDN antibodies having improved pharmaceutical characteristics. Preferably the source antibodies are modified or altered using known molecular engineering techniques to provide derived antibodies having the desired therapeutic properties.

4.1. Chimeric and Humanized Antibodies

Selected embodiments of the invention comprise murine monoclonal antibodies that immunospecifically bind to CLDN and which can be considered “source” antibodies. In selected embodiments, antibodies of the invention can be derived from such “source” antibodies through optional modification of the constant region and/or the epitope-binding amino acid sequences of the source antibody. In certain embodiments an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or domains or the entire heavy and light chain variable regions) are combined with or incorporated into an acceptor antibody sequence to provide the derivative antibody (e.g. chimeric, CDR grafted or humanized antibodies). These “derived” antibodies can be generated using genetic material from the antibody producing cell and standard molecular biological techniques as described below, such as, for example, to improve affinity for the determinant; to improve antibody stability; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification.

In one embodiment, the antibodies of the invention comprise chimeric antibodies that are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567). In some embodiments chimeric antibodies of the instant invention may comprise all or most of the selected murine heavy and light chain variable regions operably linked to human light and heavy chain constant regions. In other selected embodiments, anti-CLDN antibodies may be “derived” from the mouse antibodies disclosed herein and comprise less than the entire heavy and light chain variable regions.

In other embodiments, chimeric antibodies of the invention are “CDR-grafted” antibodies, where the CDRs (as defined using Kabat, Chothia, McCallum, etc.) are derived from a particular species or belonging to a particular antibody class or subclass, while the remainder of the antibody is largely derived from an antibody from another species or belonging to another antibody class or subclass. For use in humans, one or more selected rodent CDRs (e.g., mouse CDRs) may be grafted into a human acceptor antibody, replacing one or more of the naturally occurring CDRs of the human antibody. These constructs generally have the advantages of providing full strength human antibody functions, e.g., complement dependent cytotoxicity (CDC) and antibody-dependent cell-mediated cytotoxicity (ADCC) while reducing unwanted immune responses to the antibody by the subject. In one embodiment the CDR grafted antibodies will comprise one or more CDRs obtained from a mouse incorporated in a human framework sequence.

Similar to the CDR-grafted antibody is a “humanized” antibody. As used herein, a “humanized” antibody is a human antibody (acceptor antibody) comprising one or more amino acid sequences (e.g. CDR sequences) derived from one or more non-human antibodies (donor or source antibody). In certain embodiments, “back mutations” can be introduced into the humanized antibody, in which residues in one or more FRs of the variable region of the recipient human antibody are replaced by corresponding residues from the non-human species donor antibody. Such back mutations may to help maintain the appropriate three-dimensional configuration of the grafted CDR(s) and thereby improve affinity and antibody stability. Antibodies from various donor species may be used including, without limitation, mouse, rat, rabbit, or non-human primate. Furthermore, humanized antibodies may comprise new residues that are not found in the recipient antibody or in the donor antibody to, for example, further refine antibody performance. CDR grafted and humanized antibodies compatible with the instant invention comprising murine components from source antibodies and human components from acceptor antibodies may be provided as set forth in the Examples below.

Various art-recognized techniques can be used to determine which human sequences to use as acceptor antibodies to provide humanized constructs in accordance with the instant invention. Compilations of compatible human germline sequences and methods of determining their suitability as acceptor sequences are disclosed, for example, in Dubel and Reichert (Eds.) (2014) Handbook of Therapeutic Antibodies, 2^{nd} Edition, Wiley-Blackwell GmbH; Tomlinson, I. A. et al. (1992) J. Mol. Biol. 227:776-798; Cook, G. P. et al. (1995) Immunol. Today 16: 237-242; Chothia, D. et al. (1992) J. Mol. Biol. 227:799-817; and Tomlinson et al. (1995) EMBO J 14:4628-4638). The V-BASE directory (VBASE2—Retter et al., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, (PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

It will be appreciated that the annotated CDRs and framework sequences as provided in the appended FIGS. 2A and 2B are defined as per Kabat et al. using a proprietary Abysis database. However, as discussed herein and shown in FIGS. 2E-2H, one skilled in the art could readily identify CDRs in accordance with definitions provided by Chothia et al., ABM or MacCallum et al as well as Kabat et al. As such, anti-CLDN humanized antibodies comprising one or more CDRs derived according to any of the aforementioned systems are explicitly held to be within the scope of the instant invention.

4.2. Site-Specific Antibodies

The antibodies of the instant invention may be engineered to facilitate conjugation to a cytotoxin or other anti-cancer agent (as discussed in more detail below). It is advantageous for the antibody drug conjugate (ADC) preparation to comprise a homogenous population of ADC molecules in terms of the position of the cytotoxin on the antibody and the drug to antibody ratio (DAR). Based on the instant disclosure one skilled in the art could readily fabricate site-specific engineered constructs as described herein. As used herein a “site-specific antibody” or “site-specific construct” means an antibody, or immunoreactive fragment thereof, wherein at least one amino acid in either the heavy or light chain is deleted, altered or substituted (preferably with another amino acid) to provide at least one free cysteine. Similarly, a “site-specific conjugate” shall be held to mean an ADC comprising a site-specific antibody and at least one cytotoxin or other compound (e.g., a reporter molecule) conjugated to the unpaired or free cysteine(s). In certain embodiments the unpaired cysteine residue will comprise an unpaired intrachain cysteine residue. In other embodiments the free cysteine residue will comprise an unpaired interchain cysteine residue. In still other embodiments the free cysteine may be engineered into the amino acid sequence of the antibody (e.g., in the CH3 domain). In any event the site-specific antibody can be of various isotypes, for example, IgG, IgE, IgA or IgD; and within those classes the antibody can be of various subclasses, for example, IgG1, IgG2, IgG3 or IgG4. For IgG constructs the light chain of the antibody can comprise either a kappa or lambda isotype each incorporating a C214 that, in selected embodiments, may be unpaired due to a lack of a C220 residue in the IgG1 heavy chain.

Thus, as used herein, the terms “free cysteine” or “unpaired cysteine” may be used interchangeably unless otherwise dictated by context and shall mean any cysteine (or thiol containing) constituent (e.g., a cysteine residue) of an antibody, whether naturally present or specifically incorporated in a selected residue position using molecular engineering techniques, that is not part of a naturally occurring (or “native”) disulfide bond under physiological conditions. In certain selected embodiments the free cysteine may comprise a naturally occurring cysteine whose native interchain or intrachain disulfide bridge partner has been substituted, eliminated or otherwise altered to disrupt the naturally occurring disulfide bridge under physiological conditions thereby rendering the unpaired cysteine suitable for site-specific conjugation. In other preferred embodiments the free or unpaired cysteine will comprise a cysteine residue that is selectively placed at a predetermined site within the antibody heavy or light chain amino acid sequences. It will be appreciated that, prior to conjugation, free or unpaired cysteines may be present as a thiol (reduced cysteine), as a capped cysteine (oxidized) or as part of a non-native intra- or intermolecular disulfide bond (oxidized) with another cysteine or thiol group on the same or different molecule depending on the oxidation state of the system. As discussed in more detail below, mild reduction of the appropriately engineered antibody construct will provide thiols available for site-specific conjugation. Accordingly, in particularly preferred embodiments the free or unpaired cysteines (whether naturally occurring or incorporated) will be subject to selective reduction and subsequent conjugation to provide homogenous DAR compositions.

It will be appreciated that the favorable properties exhibited by the disclosed engineered conjugate preparations is predicated, at least in part, on the ability to specifically direct the conjugation and largely limit the fabricated conjugates in terms of conjugation position and the absolute DAR value of the composition. Unlike most conventional ADC preparations the present invention need not rely entirely on partial or total reduction of the antibody to provide random conjugation sites and relatively uncontrolled generation of DAR species. Rather, in certain aspects the present invention preferably provides one or more predetermined unpaired (or free) cysteine sites by engineering the targeting antibody to disrupt one or more of the naturally occurring (i.e., “native”) interchain or intrachain disulfide bridges or to introduce a cysteine residue at any position. To this end it will be appreciated that, in selected embodiments, a cysteine residue may be incorporated anywhere along the antibody (or immunoreactive fragment thereof) heavy or light chain or appended thereto using standard molecular engineering techniques. In other preferred embodiments disruption of native disulfide bonds may be effected in combination with the introduction of a non-native cysteine (which will then comprise the free cysteine) that may then be used as a conjugation site.

In certain embodiments the engineered antibody comprises at least one amino acid deletion or substitution of an intrachain or interchain cysteine residue. As used herein “interchain cysteine residue” means a cysteine residue that is involved in a native disulfide bond either between the light and heavy chain of an antibody or between the two heavy chains of an antibody while an “intrachain cysteine residue” is one naturally paired with another cysteine in the same heavy or light chain. In one embodiment the deleted or substituted interchain cysteine residue is involved in the formation of a disulfide bond between the light and heavy chain. In another embodiment the deleted or substituted cysteine residue is involved in a disulfide bond between the two heavy chains. In a typical embodiment, due to the complementary structure of an antibody, in which the light chain is paired with the VH and CH1 domains of the heavy chain and wherein the CH2 and CH3 domains of one heavy chain are paired with the CH2 and CH3 domains of the complementary heavy chain, a mutation or deletion of a single cysteine in either the light chain or in the heavy chain would result in two unpaired cysteine residues in the engineered antibody.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In selected embodiments an interchain cysteine is replaced with a serine.

In some embodiments contemplated by the invention the deleted or substituted cysteine residue is on the light chain (either kappa or lambda) thereby leaving a free cysteine on the heavy chain. In other embodiments the deleted or substituted cysteine residue is on the heavy chain leaving the free cysteine on the light chain constant region. Upon assembly it will be appreciated that deletion or substitution of a single cysteine in either the light or heavy chain of an intact antibody results in a site-specific antibody having two unpaired cysteine residues.

In one embodiment the cysteine at position 214 (C214) of the IgG light chain (kappa or lambda) is deleted or substituted. In another embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. In another embodiment C214 in the light chain is substituted with serine (C214S) to provide the desired free cysteine in the heavy chain. Such site-specific constructs are described in more detail in the Examples below. A summary of compatible site-specific constructs is shown in Table 2 immediately below where numbering is generally according to the Eu index as set forth in Kabat, WT stands for “wild-type” or native constant region sequences without alterations and delta (4) designates the deletion of an amino acid residue (e.g., C2144 indicates that the cysteine residue at position 214 has been deleted).

TABLE 2

| | Antibody | | |
|---|---|---|---|
| Designation | Component | Alteration | SEQ ID NO: |
| ss1 | Heavy Chain | C220S | 2 |
| | Light Chain | WT | 4 and 7 |
| ss2 | Heavy Chain | C220Δ | 3 |
| | Light Chain | WT | 4 and 7 |
| ss3 | Heavy Chain | WT | 1 |
| | Light Chain | C214Δ | 6 and 9 |
| ss4 | Heavy Chain | WT | 1 |
| | Light Chain | C214S | 5 and 8 |

Exemplary engineered light and heavy chain constant regions compatible with site specific constructs of the instant invention are set forth immediately below where SEQ ID NOS: 2 and 3 comprise, respectively, C220S IgG1 and C2204 IgG1 heavy chain constant regions, SEQ ID NOS: 5 and 6 comprise, respectively, C214S and C2144 kappa light chain constant regions and SEQ ID NOS: 8 and 9 comprise, respectively, exemplary C214S and 02144 lambda light chain constant regions. In each case the site of the altered or deleted amino acid (along with the flanking residues) is underlined.

(SEQ ID NO: 2)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTIPPVLDSDGSFFLYSKLTVDKSRW
QQGNVFSCSVMHEALHNHYTQKSLSLSPG
(SEQ ID NO: 3)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTIPPVLDSDGSFFLYSKLTVDKSRWQ
QGNVFSCSVMHEALHNHYTQKSLSLSPG
(SEQ ID NO: 5)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGES
(SEQ ID NO: 6)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGE
(SEQ ID NO: 8)
QPKANPTVTLFPPSSEELQANKATLVCLISDFYPGAVTVAWKADGSPVKA
GVETTKPSKQSNNKYAASSYLSLTPEQWKSHRSYSCQVTHEGSTVEKTVA
PTESS
(SEQ ID NO: 9)
QPKANPTVTLFPPSSEELQANKATLVCLISDFYPGAVTVAWKADGSPVKA
GVETTKPSKQSNNKYAASSYLSLTPEQWKSHRSYSCQVTHEGSTVEKTVA
PTES

As discussed above each of the heavy and light chain variants may be operably associated with the disclosed heavy and light chain variable regions (or derivatives thereof such as humanized or CDR grafted constructs) to provide site-specific anti-CLDN antibodies as disclosed herein. Such engineered antibodies are particularly compatible for use in the disclosed ADCs.

With regard to the introduction or addition of a cysteine residue or residues to provide a free cysteine (as opposed to disrupting a native disulfide bond) compatible position(s) on the antibody or antibody fragment may readily be discerned by one skilled in the art. Accordingly, in selected embodiments the cysteine(s) may be introduced in the CH1 domain, the CH2 domain or the CH3 domain or any combination thereof depending on the desired DAR, the antibody construct, the selected payload and the antibody target. In other preferred embodiments the cysteines may be introduced into a kappa or lambda CL domain and, in particularly preferred embodiments, in the c-terminal region of the CL domain. In each case other amino acid residues proximal to the site of cysteine insertion may be altered, removed or substituted to facilitate molecular stability, conjugation efficiency or provide a protective environment for the payload once it is attached. In particular embodiments, the substituted residues occur at any accessible sites of the antibody. By substituting such surface residues with cysteine, reactive thiol groups are thereby positioned at readily accessible sites on the antibody and may be selectively reduced as described further herein. In particular embodiments, the substituted residues occur at accessible sites of the antibody. By substituting those residues with cysteine, reactive thiol groups are thereby positioned at accessible sites of the antibody and may be used to selectively conjugate the antibody. In certain embodiments, any one or more of the following residues may be substituted with cysteine: V205 (Kabat numbering) of the light chain; A118 (Eu numbering) of the heavy chain; and S400 (Eu numbering) of the heavy chain Fc region. Additional substitution positions and methods of fabricating compatible site-specific antibodies are set forth in U.S. Pat. No. 7,521,541 which is incorporated herein in its entirety.

The strategy for generating antibody drug conjugates with defined sites and stoichiometries of drug loading, as disclosed herein, is broadly applicable to all anti-CLDN antibodies as it primarily involves engineering of the conserved constant domains of the antibody. As the amino acid sequences and native disulfide bridges of each class and subclass of antibody are well documented, one skilled in the art could readily fabricate engineered constructs of various antibodies without undue experimentation and, accordingly, such constructs are expressly contemplated as being within the scope of the instant invention. This is particularly true of site-specific constructs comprising all or part of the heavy and light chain variable region amino acid sequences as set forth in the instant disclosure.

4.3. Constant Region Modifications and Altered Glycosylation

Selected embodiments of the present invention may also comprise substitutions or modifications of the constant region (i.e. the Fc region), including without limitation, amino acid residue substitutions, mutations and/or modifications, which result in a compound with preferred characteristics including, but not limited to: altered pharmacokinetics, increased serum half-life, increase binding affinity, reduced immunogenicity, increased production, altered Fc ligand binding to an Fc receptor (FcR), enhanced or reduced ADCC or CDC, altered glycosylation and/or disulfide bonds and modified binding specificity.

Compounds with improved Fc effector functions can be generated, for example, through changes in amino acid residues involved in the interaction between the Fc domain and an Fc receptor (e.g., FcγRI, FcγRIIA and B, FcγRIII and FcRn), which may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311). With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001).

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

Still other embodiments comprise one or more engineered glycoforms, e.g., a site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the antibody for a target or facilitating production of the antibody. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTIII)), by expressing a molecule comprising an Fc region in various organisms or cell lines from various organisms or by modifying carbohydrate(s) after the molecule comprising Fc region has been expressed (see, for example, WO 2012/117002).

4.4. Fragments

Regardless of which form of antibody (e.g. chimeric, humanized, etc.) is selected to practice the invention it will be appreciated that immunoreactive fragments, either by themselves or as part of an antibody drug conjugate, of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody that immunospecifically binds or reacts with a selected antigen or immunogenic determinant thereof or competes with the intact antibody from which the fragments were derived for specific antigen binding.

Exemplary site-specific fragments include: variable light chain fragments (VL), an variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency). Such antibody fragments may further be engineered to comprise one or more free cysteines as described herein.

In other embodiments, an antibody fragment is one that comprises the Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

4.5. Multivalent Constructs

In other embodiments, the antibodies and conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

Multivalent antibodies may immunospecifically bind to different epitopes of the desired target molecule or may immunospecifically bind to both the target molecule as well as a heterologous epitope, such as a heterologous polypeptide or solid support material. While selected embodiments may only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

In yet other embodiments, antibody variable domains with the desired binding specificities (antibody-antigen combining sites) are fused to immunoglobulin constant domain sequences, such as an immunoglobulin heavy chain constant domain comprising at least part of the hinge, CH2, and/or CH3 regions, using methods well known to those of ordinary skill in the art.

5. Recombinant Production of Antibodies

Antibodies and fragments thereof may be produced or modified using genetic material obtained from antibody producing cells and recombinant technology (see, for example; Dubel and Reichert (Eds.) (2014) Handbook of Therapeutic Antibodies, 2^{nd} Edition, Wiley-Blackwell GmbH; Sambrook and Russell (Eds.) (2000) Molecular Cloning: A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611).

Another aspect of the invention pertains to nucleic acid molecules that encode the antibodies of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In selected embodiments the nucleic acid is a cDNA molecule.

Nucleic acids of the invention can be obtained using standard molecular biology techniques. For antibodies expressed by hybridomas (e.g., hybridomas prepared as described in the Examples below), cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

DNA fragments encoding VH and VL segments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, means that the two DNA fragments are joined such that the amino acid sequences encoded by the two DNA fragments remain in-frame.

The isolated DNA encoding the VH region can be converted to a full-length heavy chain gene by operatively linking (or operatively associating) the VH-encoding DNA to another DNA molecule encoding heavy chain constant regions (CH1, CH2 and CH3 in the case of IgG1). The sequences of human heavy chain constant region genes are known in the art (see e.g., Kabat, et al. (1991) (supra)) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary kappa light chain constant region amino acid sequence compatible with the present invention is set forth immediately below:

(SEQ ID NO: 4)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

An exemplary lambda light chain constant region amino acid sequence compatible with the present invention is set forth immediately below:

(SEQ ID NO: 7)
QPKANPTVTLFPPSSEELQANKATLVCLISDFYPGAVTVAWKADGSPVKA
GVETTKPSKQSNNKYAASSYLSLTPEQWKSHRSYSCQVTHEGSTVEKTVA
PTECS

Similarly, an exemplary IgG1 heavy chain constant region amino acid sequence compatible with the present invention is set forth immediately below:

(SEQ ID NO: 1)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTIPPVLDSDGSFFLYSKLTVDKSRW
QQGNVFSCSVMHEALHNHYTQKSLSLSPG.

For a Fab fragment heavy chain gene, the VH-encoding DNA can be operatively linked to another DNA molecule encoding only the heavy chain CH1 constant region.

Isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region.

Contemplated herein are certain polypeptides (e.g. antigens or antibodies) that exhibit “sequence identity”, sequence similarity” or “sequence homology” to the polypeptides of the invention. For example, a derived humanized antibody VH or VL domain may exhibit a sequence similarity with the source (e.g., murine) or acceptor (e.g., human) VH or VL domain. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any kind of cellular system that can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO-S, HEK293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under selected conditions. The GS system is discussed in whole or part in connection with EP 0 216 846, EP 0 256 055, EP 0 323 997 and EP 0 338 841 and U.S. Pat. Nos. 5,591,639 and 5,879,936. Another compatible expression system for the development of stable cell lines is the Freedom™ CHO-S Kit (Life Technologies).

Once an antibody of the invention has been produced by recombinant expression or any other of the disclosed techniques, it may be purified or isolated by methods known in the art in that it is identified and separated and/or recovered from its natural environment and separated from contaminants that would interfere with diagnostic or therapeutic uses for the antibody or related ADC. Isolated antibodies include antibodies in situ within recombinant cells.

These isolated preparations may be purified using various art-recognized techniques, such as, for example, ion exchange and size exclusion chromatography, dialysis, diafiltration, and affinity chromatography, particularly Protein A or Protein G affinity chromatography. Compatible methods are discussed more fully in the Examples below.

6. Post-Production Selection

No matter how obtained, antibody-producing cells (e.g., hybridomas, yeast colonies, etc.) may be selected, cloned and further screened for desirable characteristics including, for example, robust growth, high antibody production and desirable antibody characteristics such as high affinity for the antigen of interest. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M⁻¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

### IV. CHARACTERISTICS OF ANTIBODIES

In certain embodiments, antibody-producing cells (e.g., hybridomas or yeast colonies) may be selected, cloned and further screened for favorable properties including, for example, robust growth, high antibody production and, as discussed in more detail below, desirable site-specific antibody characteristics. In other cases characteristics of the antibody may be imparted by selecting a particular antigen (e.g., a specific CLDN isoform) or immunoreactive fragment of the target antigen for inoculation of the animal. In still other embodiments the selected antibodies may be engineered as described above to enhance or refine immunochemical characteristics such as affinity or pharmacokinetics.

A. Neutralizing Antibodies

In certain embodiments, the antibodies or antibody conjugates will comprise “neutralizing” antibodies or derivatives or fragments thereof. That is, the present invention may comprise antibody molecules that bind specific domains or epitopes and are capable of blocking, reducing or inhibiting the biological activity of CLDN6. More generally the term “neutralizing antibody” refers to an antibody that binds to or interacts with a target molecule or ligand and prevents binding or association of the target molecule to a binding partner such as a receptor or substrate, thereby interrupting a biological response that otherwise would result from the interaction of the molecules.

It will be appreciated that competitive binding assays known in the art may be used to assess the binding and specificity of an antibody or immunologically functional fragment or derivative thereof. With regard to the instant invention an antibody or fragment will be held to inhibit or reduce binding of CLDN to a binding partner or substrate when an excess of antibody reduces the quantity of binding partner bound to CLDN by at least about 20%, 30%, 40%, 50%, 60%, 70%, 80%, 85%, 90%, 95%, 97%, 99% or more as measured, for example, by target molecule activity or in an in vitro competitive binding assay. In the case of antibodies to CLDN for example, a neutralizing antibody or antagonist will preferably alter target molecule activity by at least about 20%, 30%, 40%, 50%, 60%, 70%, 80%, 85%, 90%, 95%, 97%, 99% or more. It will be appreciated that this modified activity may be measured directly using art-recognized techniques or may be measured by the impact the altered activity has downstream (e.g., oncogenesis or cell survival).

B. Internalizing Antibodies

In certain embodiments the antibodies may comprise internalizing antibodies such that the antibody will bind to a determinant and will be internalized (along with any conjugated pharmaceutically active moiety) into a selected target cell including tumorigenic cells. The number of antibody molecules internalized may be sufficient to kill an antigen-expressing cell, especially an antigen-expressing tumorigenic cell. Depending on the potency of the antibody or, in some instances, antibody drug conjugate, the uptake of a single antibody molecule into the cell may be sufficient to kill the target cell to which the antibody binds. With regard to the instant invention there is evidence that a substantial portion of expressed CLDN protein remains associated with the tumorigenic cell surface, thereby allowing for localization and internalization of the disclosed antibodies or ADCs. In selected embodiments such antibodies will be associated with, or conjugated to, one or more drugs that kill the cell upon internalization. In some embodiments the ADCs of the instant invention will comprise an internalizing site-specific ADC.

As used herein, an antibody that “internalizes” is one that is taken up (along with any conjugated cytotoxin) by a target cell upon binding to an associated determinant. The number of such ADCs internalized will preferably be sufficient to kill the determinant-expressing cell, especially a determinant expressing cancer stem cell. Depending on the potency of the cytotoxin or ADC as a whole, in some instances the uptake of a few antibody molecules into the cell is sufficient to kill the target cell to which the antibody binds. For example, certain drugs such as PBDs or calicheamicin are so potent that the internalization of a few molecules of the toxin conjugated to the antibody is sufficient to kill the target cell. Whether an antibody internalizes upon binding to a mammalian cell can be determined by various art-recognized assays (e.g., saporin assays such as Mab-Zap and Fab-Zap; Advanced Targeting Systems) including those described in the Examples below. Methods of detecting whether an antibody internalizes into a cell are also described in U.S. Pat. No. 7,619,068.

C. Depleting Antibodies

In other embodiments the antibodies of the invention are depleting antibodies. The term “depleting” antibody refers to an antibody that preferably binds to an antigen on or near the cell surface and induces, promotes or causes the death of the cell (e.g., by CDC, ADCC or introduction of a cytotoxic agent). In embodiments, the selected depleting antibodies will be conjugated to a cytotoxin.

Preferably a depleting antibody will be able to kill at least 20%, 30%, 40%, 50%, 60%, 70%, 80%, 85%, 90%, 95%, 97%, or 99% of CLDN-expressing cells in a defined cell population. The term “apparent 1050”, as used herein, refers to the concentration at which a primary antibody linked to a toxin kills 50 percent of the cells expressing the antigen(s) recognized by the primary antibody. The toxin can be directly conjugated to the primary antibody, or can be associated with the primary antibody via a secondary antibody or antibody fragment that recognizes the primary antibody, and which secondary antibody or antibody fragment is directly conjugated to a toxin. Preferably a depleting antibody will have an 1050 of less than 5 μM. less than 1 μM, less than 100 nM, less than 50 nM, less than 30 nM, less than 20 nM, less than 10 nM, less than 5 nM, less than 2 nM or less than 1 nM. In some embodiments the cell population may comprise enriched, sectioned, purified or isolated tumorigenic cells, including cancer stem cells. In other embodiments the cell population may comprise whole tumor samples or heterogeneous tumor extracts that comprise cancer stem cells. Standard biochemical techniques may be used to monitor and quantify the depletion of tumorigenic cells in accordance with the teachings herein.

D. Binding Affinity

Disclosed herein are antibodies that have a high binding affinity for a specific determinant e.g. CLDN. The term “K_{D}” refers to the dissociation constant of a particular antibody-antigen interaction. An antibody of the invention can immunospecifically bind its target antigen when the dissociation constant K_{D} (k_{off}/kₒₙ) is ≤10⁻⁷ M. The antibody specifically binds antigen with high affinity when the K_{D} is ≤5×10⁻⁹ M, and with very high affinity when the K_{D} is ≤5×10⁻¹⁰ M. In one embodiment of the invention, the antibody has a K_{D} of ≤10⁻⁹M and an off-rate of about 1×10⁻⁴/sec. In one embodiment of the invention, the off-rate is <1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≤2×10⁻¹⁰ M. Still other selected embodiments of the invention comprise antibodies that have a K_{D} (k_{off}/kₒₙ) of less than 10⁻⁶ M, less than 5×10⁻⁶M, less than 10⁻⁷M, less than 5×10⁻⁷M, less than 10⁻⁸ M, less than 5×10⁻⁸ M, less than 10⁻⁹M, less than 5×10⁻⁹M, less than 10⁻¹⁰M, less than 5×10⁻¹⁰ M, less than 10⁻¹¹ M, less than 5×10⁻¹¹ M, less than 10⁻¹⁴ M, less than 5×10⁻¹² M, less than 10⁻¹³ M, less than 5×10⁻¹³ M, less than 10⁻¹⁴M, less than 5×10⁻¹⁴ M, less than 10⁻¹⁵M or less than 5×10⁻¹⁵M.

In certain embodiments, an antibody of the invention that immunospecifically binds to a determinant e.g. CLDN may have an association rate constant or kₒₙ (or kₐ) rate (antibody+antigen (Ag)^{k}ₒₙ←antibody-Ag) of at least 10⁵ M⁻¹s⁻¹, at least 2×10⁵ M⁻¹s⁻¹, at least 5×10⁵ M⁻¹s⁻¹, at least 10⁶M⁻¹s⁻¹, at least 5×10⁶M⁻¹s⁻¹, at least 10⁷M⁻¹s⁻¹, at least 5×10⁷M⁻¹s⁻¹, or at least 10⁸ M⁻¹s⁻¹.

In another embodiment, an antibody of the invention that immunospecifically binds to a determinant e.g. CLDN may have a disassociation rate constant or k_{off} (or k_{d}) rate (antibody+antigen (Ag)^{k}_{off}←antibody-Ag) of less than 10⁻¹ s⁻¹, less than 5×10¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁴ s⁻¹, less than 10⁻⁵ s⁻¹, less than 5×10⁻⁵ s⁻¹, less than 10⁻⁶ s⁻¹, less than 5×10⁻⁶ s⁻¹ less than 10⁻⁷ s⁻¹, less than 5×10⁻⁷ s⁻¹, less than 10⁻⁸ s⁻¹, less than 5×10⁻⁸ s⁻¹, less than 10⁻⁶ s⁻¹, less than 5×10⁻⁶ s⁻¹ or less than 10⁻¹⁰ s⁻¹.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry.

The term “apparent binding affinity” as used herein, refers to the apparent binding of an antibody to its target antigen when the antigen is overexpressed on the surface of a cell. The apparent binding affinity of an antibody for an antigen is described herein as an “apparent EC50”, which is the concentration of antibody at which 50% maximal binding to cells overexpressing the antigen occurs. In one embodiment, two antibodies can be said to have “substantially the same” apparent binding affinity for an antigen, with >99% confidence, if they have apparent EC50 values that do not differ from one another by more than 45%, by more than 40%, by more than 35%, by more than 30%, by more than 25%, by more than 20%, by more than 10% or by more than 5%. In another embodiment an antibody that binds multiple target antigens, e.g. is multireactive towards one or more CLDN proteins, can be said to have “substantially the same” apparent binding affinity for the multiple antigens, with >99% confidence, if the apparent EC50 values of the antibody for each of the antigens do not differ from one another by more than 45%, by more than 40%, by more than 35%, by more than 30%, by more than 25%, by more than 20%, by more than 10% or by more than 5%. Since the assays used to determine the apparent binding affinity of an antibody for an antigen typically utilize cells overexpressing the antigen and which are exposed to antibodies under presumed equilibrium or near equilibrium conditions, the apparent EC50 value is reflective of the avidity, or combined or accumulated strength of multiple apparent binding affinities. Thus, in a related embodiment two antibodies will share substantially the same avidity for a target cell line expressing the antigen, with >99% confidence, if their apparent binding affinities for the cell line, expressed as apparent EC50 values, do not differ from one another by more than 45%, by more than 40%, by more than 35%, by more than 30%, by more than 25%, by more than 20%, by more than 10% or by more than 5%. Similarly an antibody that binds multiple target antigens, e.g. is multireactive towards one or more CLDN proteins, can be said to have substantially the same avidity for multiple antigens, with >99% confidence, if the apparent EC50 values for each of the antigens do not differ from one another by more than 45%, by more than 40%, by more than 35%, by more than 30%, by more than 25%, by more than 20%, by more than 10% or by more than 5%.

E. Binning and Epitope Mapping

As used herein, the term “binning” refers to methods used to group antibodies into “bins” based on their antigen binding characteristics and whether they compete with each other. The initial determination of bins may be further refined and confirmed by epitope mapping and other techniques as described herein. However it will be appreciated that empirical assignment of antibodies to individual bins provides information that may be indicative of the therapeutic potential of the disclosed antibodies.

One can determine whether a selected reference antibody (or fragment thereof) competes for binding with a second test antibody (i.e., is in the same bin) by using methods known in the art and set forth in the Examples herein. In one embodiment, a reference antibody is associated with the CLDN antigen under saturating conditions and then the ability of a secondary or test antibody to bind to CLDN is determined using standard immunochemical techniques. If the test antibody is able to substantially bind to CLDN at the same time as the reference anti-CLDN antibody, then the secondary or test antibody binds to a different epitope than the primary or reference antibody. However, if the test antibody is not able to substantially bind to CLDN at the same time, then the test antibody binds to the same epitope, an overlapping epitope, or an epitope that is in close proximity (at least sterically) to the epitope bound by the primary antibody. That is, the test antibody competes for antigen binding and is in the same bin as the reference antibody.

The term “compete” or “competing antibody” when used in the context of the disclosed antibodies means competition between antibodies as determined by an assay in which a test antibody or immunologically functional fragment being tested inhibits specific binding of a reference antibody to a common antigen. Typically, such an assay involves the use of purified antigen (e.g., CLDN or a domain or fragment thereof) bound to a solid surface or cells, an unlabeled test antibody and a labeled reference antibody. Competitive inhibition is measured by determining the amount of label bound to the solid surface or cells in the presence of the test antibody. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more. Conversely, when the reference antibody is bound it will preferably inhibit binding of a subsequently added test antibody (i.e., an anti-CLDN antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

Similarly binning techniques comprising surface plasmon resonance are compatible with the instant invention. As used herein “surface plasmon resonance,” refers to an optical phenomenon that allows for the analysis of real-time specific interactions by detection of alterations in protein concentrations within a biosensor matrix. Using commercially available equipment such as the BIAcore™ 2000 system it may readily be determined if selected antibodies compete with each other for binding to a defined antigen.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In some embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which that group of antibodies binds. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds. Antibodies disclosed herein may be characterized in terms of the discrete epitope with which they associate. An “epitope” is the portion(s) of a determinant to which the antibody or immunoreactive fragment specifically binds. Immunospecific binding can be confirmed and defined based on binding affinity, as described above, or by the preferential recognition by the antibody of its target antigen in a complex mixture of proteins and/or macromolecules (e.g. in competition assays). A “linear epitope”, is formed by contiguous amino acids in the antigen that allow for immunospecific binding of the antibody. The ability to preferentially bind linear epitopes is typically maintained even when the antigen is denatured. Conversely, a “conformational epitope”, usually comprises non-contiguous amino acids in the antigen's amino acid sequence but, in the context of the antigen's secondary, tertiary or quaternary structure, are sufficiently proximate to be bound concomitantly by a single antibody. When antigens with conformational epitopes are denatured, the antibody will typically no longer recognize the antigen. An epitope (contiguous or non-contiguous) typically includes at least 3, and more usually, at least 5 or 8-10 or 12-20 amino acids in a unique spatial conformation.

In certain embodiments fine epitope mapping can be performed using phage or yeast display. Other compatible epitope mapping techniques include alanine scanning mutants, peptide blots (Reineke, 2004, PMID: 14970513), or peptide cleavage analysis. In addition, methods such as epitope excision, epitope extraction and chemical modification of antigens can be employed (Tomer, 2000, PMID: 10752610) using enzymes such as proteolytic enzymes (e.g., trypsin, endoproteinase Glu-C, endoproteinase Asp-N, chymotrypsin, etc.); chemical agents such as succinimidyl esters and their derivatives, primary amine-containing compounds, hydrazines and carbohydrazines, free amino acids, etc. In another embodiment Modification-Assisted Profiling, also known as Antigen Structure-based Antibody Profiling (ASAP) can be used to categorize large numbers of monoclonal antibodies directed against the same antigen according to the similarities of the binding profile of each antibody to chemically or enzymatically modified antigen surfaces (U.S.P.N. 2004/0101920).

Once a desired epitope on an antigen is determined, it is possible to generate additional antibodies to that epitope, e.g., by immunizing with a peptide comprising the selected epitope using techniques described herein.

### V. ANTIBODY CONJUGATES

In some embodiments the antibodies of the invention may be conjugated with pharmaceutically active or diagnostic moieties to form an “antibody drug conjugate” (ADC) or “antibody conjugate”. The term “conjugate” is used broadly and means the covalent or non-covalent association of any pharmaceutically active or diagnostic moiety with an antibody of the instant invention regardless of the method of association. In certain embodiments the association is effected through a lysine or cysteine residue of the antibody. In some embodiments the pharmaceutically active or diagnostic moieties may be conjugated to the antibody via one or more site-specific free cysteine(s). The disclosed ADCs may be used for therapeutic and diagnostic purposes.

The ADCs of the instant invention may be used to deliver cytotoxins or other payloads to the target location (e.g., tumorigenic cells expressing CLDN). As set forth herein the terms “drug” or “warhead” may be used interchangeably and will mean a biologically active or detectable molecule or drug, including anti-cancer agents or cytotoxins as described below. A “payload” may comprise a “drug” or “warhead” in combination with an optional linker compound. The warhead on the conjugate may comprise peptides, proteins or prodrugs which are metabolized to an active agent in vivo, polymers, nucleic acid molecules, small molecules, binding agents, mimetic agents, synthetic drugs, inorganic molecules, organic molecules and radioisotopes. In a preferred embodiment, the disclosed ADCs will direct the bound payload to the target site in a relatively unreactive, non-toxic state before releasing and activating the warhead (e.g., PBDS 1-5 as disclosed herein). This targeted release of the warhead is preferably achieved through stable conjugation of the payloads (e.g., via one or more cysteines on the antibody) and the relatively homogeneous composition of the ADC preparations which minimize over-conjugated toxic ADC species. Coupled with drug linkers that are designed to largely release the warhead once it has been delivered to the tumor site, the conjugates of the instant invention can substantially reduce undesirable non-specific toxicity. This advantageously provides for relatively high levels of the active cytotoxin at the tumor site while minimizing exposure of non-targeted cells and tissue thereby providing an enhanced therapeutic index.

It will be appreciated that, while some embodiments of the invention comprise payloads incorporating therapeutic moieties (e.g., cytotoxins), other payloads incorporating diagnostic agents and biocompatible modifiers may benefit from the targeted release provided by the disclosed conjugates. Accordingly, any disclosure directed to exemplary therapeutic payloads is also applicable to payloads comprising diagnostic agents or biocompatible modifiers as discussed herein unless otherwise dictated by context. The selected payload may be covalently or non-covalently linked to, the antibody and exhibit various stoichiometric molar ratios depending, at least in part, on the method used to effect the conjugation.

Conjugates of the instant invention may be generally represented by the formula:

Ab-[L-D]n or a pharmaceutically acceptable salt thereof wherein:
a) Ab comprises an anti-CLDN antibody;
b) L comprises an optional linker;
c) D comprises a drug; and
d) n is an integer from about 1 to about 20.

Those of skill in the art will appreciate that conjugates according to the aforementioned formula may be fabricated using a number of different linkers and drugs and that conjugation methodology will vary depending on the selection of components. As such, any drug or drug linker compound that associates with a reactive residue (e.g., cysteine or lysine) of the disclosed antibodies are compatible with the teachings herein. Similarly, any reaction conditions that allow for conjugation (including site-specific conjugation) of the selected drug to an antibody are within the scope of the present invention. Notwithstanding the foregoing, some preferred embodiments of the instant invention comprise selective conjugation of the drug or drug linker to free cysteines using stabilization agents in combination with mild reducing agents as described herein. Such reaction conditions tend to provide more homogeneous preparations with less non-specific conjugation and contaminants and correspondingly less toxicity.

A. Warheads

1. Therapeutic Agents

The antibodies of the invention may be conjugated, linked or fused to or otherwise associated with a pharmaceutically active moiety which is a therapeutic moiety or a drug such as an anti-cancer agent including, but not limited to, cytotoxic agents (or cytotoxins), cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Exemplary anti-cancer agents or cytotoxins (including homologs and derivatives thereof) comprise 1-dehydrotestosterone, anthramycins, actinomycin D, bleomycin, calicheamicins (including n-acetyl calicheamicin), colchicin, cyclophosphamide, cytochalasin B, dactinomycin (formerly actinomycin), dihydroxy anthracin, dione, duocarmycin, emetine, epirubicin, ethidium bromide, etoposide, glucocorticoids, gramicidin D, lidocaine, maytansinoids such as DM-1 and DM-4 (Immunogen), benzodiazepine derivatives (Immunogen), mithramycin, mitomycin, mitoxantrone, paclitaxel, procaine, propranolol, puromycin, tenoposide, tetracaine and pharmaceutically acceptable salts or solvates, acids or derivatives of any of the above.

Additional compatible cytotoxins comprise dolastatins and auristatins, including monomethyl auristatin E (MMAE) and monomethyl auristatin F (MMAF) (Seattle Genetics), amanitins such as alpha-amanitin, beta-amanitin, gamma-amanitin or epsilon-amanitin (Heidelberg Pharma), DNA minor groove binding agents such as duocarmycin derivatives (Syntarga), alkylating agents such as modified or dimeric pyrrolobenzodiazepines (PBD), mechlorethamine, thioepa, chlorambucil, melphalan, carmustine (BCNU), lomustine (CCNU), cyclothosphamide, busulfan, dibromomannitol, streptozotocin, mitomycin C and cisdichlorodiamine platinum (II) (DDP) cisplatin, splicing inhibitors such as meayamycin analogs or derivatives (e.g., FR901464 as set forth in U.S. Pat. No. 7,825,267), tubular binding agents such as epothilone analogs and tubulysins, paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (formerly daunomycin) and doxorubicin and pharmaceutically acceptable salts or solvates, acids or derivatives of any of the above.

In selected embodiments the antibodies of the instant invention may be associated with anti-CD3 binding molecules to recruit cytotoxic T-cells and have them target tumorigenic cells (BiTE technology; see e.g., Fuhrmann et. al. (2010) Annual Meeting of AACR Abstract No. 5625).

In further embodiments ADCs of the invention may comprise cytotoxins comprising therapeutic radioisotopes conjugated using appropriate linkers. Exemplary radioisotopes that may be compatible with such embodiments include, but are not limited to, iodine (¹³¹I, ¹²⁵I, ¹²³I, ¹²¹I), carbon (¹⁴C), copper (⁶²Cu, ⁶⁴Cu, ⁶⁷Cu), sulfur (³⁵S), radium (²²³R), tritium (³H), indium (¹¹⁵In, ¹¹³In, ¹¹²In, ¹¹¹In), bismuth (²¹²Bi, ²¹³ Bi), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷SC, ¹⁸⁶Re, ¹⁸⁵Re, ¹⁴² Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, ¹¹⁷Sn, ⁷⁶Br, ²¹¹At and ²²⁵Ac. Other radionuclides are also available as diagnostic and therapeutic agents, especially those in the energy range of 60 to 4,000 keV.

In other selected embodiments the ADCs of the instant invention will be conjugated to a cytotoxic benzodiazepine derivative warhead. Compatible benzodiazepine derivatives (and optional linkers) that may be conjugated to the disclosed antibodies are described, for example, in U.S. Pat. No. 8,426,402 and PCT filings WO2012/128868 and WO2014/031566. As with PBDs, compatible benzodiazepine derivatives are believed to bind in the minor grove of DNA and inhibit nucleic acid synthesis. Such compounds reportedly have potent antitumor properties and, as such, are particularly suitable for use in the ADCs of the instant invention.

In some embodiments, the ADCs of the invention may comprise PBDs, and pharmaceutically acceptable salts or solvates, acids or derivatives thereof, as warheads. PBDs are alkylating agents that exert antitumor activity by covalently binding to DNA in the minor groove and inhibiting nucleic acid synthesis. PBDs have been shown to have potent antitumor properties while exhibiting minimal bone marrow depression. PBDs compatible with the invention may be linked to an antibody using several types of linkers (e.g., a peptidyl linker comprising a maleimido moiety with a free sulfhydryl), and in certain embodiments are dimeric in form (i.e., PBD dimers). Compatible PBDs (and optional linkers) that may be conjugated to the disclosed antibodies are described, for example, in U.S. Pat. Nos. 6,362,331, 7,049,311, 7,189,710, 7,429,658, 7,407,951, 7,741,319, 7,557,099, 8,034,808, 8,163,736, 2011/0256157 and PCT filings WO2011/130613, WO2011/128650, WO2011/130616, WO2014/057073 and WO2014/057074. Examples of PBD compounds compatible with the instant invention are discussed in more detail immediately below.

With regard to the instant invention PBDs have been shown to have potent antitumor properties while exhibiting minimal bone marrow depression. PBDs compatible with the present invention may be linked to the CLDN targeting agent using any one of several types of linker (e.g., a peptidyl linker comprising a maleimido moiety with a free sulfhydryl) and, in certain embodiments are dimeric in form (i.e., PBD dimers), PBDs are of the general structure:

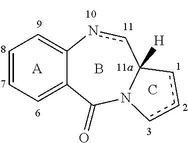

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic center responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove enables them to interfere with DNA processing and act as cytotoxic agents. As alluded to above, in order to increase their potency PBDs are often used in a dimeric form which may be conjugated to anti-CLDN antibodies as described herein.

In certain embodiments of the instant invention compatible PBDs that may be conjugated to the disclosed modulators are described in U.S.P.N. 2011/0256157. This disclosure provides PBD dimers, (i.e. those comprising two PBD moieties) that are shown to have certain advantageous properties. In this regard selected ADCs of the present invention comprise PBD toxins having the formula (AB) or (AC):

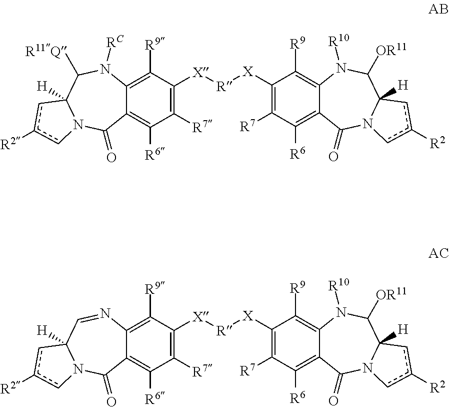

wherein:
the dotted lines indicate the optional presence of a double bond between C1 and C2 or C2 and C3;
R² is independently selected from H, OH, ═O, ═CH₂, CN, R, OR, ═CH—R^{D}, ═C(R^{D})₂, O—SO₂—R, CO₂R and COR, and optionally further selected from halo or dihalo;
where R^{D} is independently selected from R, CO₂R, COR, CHO, CO₂H, and halo;
R⁶ and R⁹ are independently selected from H, R, OH, OR, SH, SR, NH₂, NHR, NRR′, NO₂, Me₃Sn and halo;
R⁷ is independently selected from H, R, OH, OR, SH, SR, NH₂, NHR, NRR′, NO₂, Me₃Sn and halo;
R¹⁰ is a linker connected to a CLDN antibody or fragment or derivative thereof, as described herein;
Q is independently selected from O, S and NH;
R¹¹ is either H, or R or, where Q is O, R¹¹ may be SO₃M, where M is a metal cation;
X is selected from O, S, or N(H) and in selected embodiments comprises O;
R″ is a C₃₋₁₂ alkylene group, which chain may be interrupted by one or more heteroatoms (e.g., O, S, N(H), NMe and/or aromatic rings, e.g. benzene or pyridine, which rings are optionally substituted);
R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring; and

wherein R^{2″}, R^{6″}, R^{7″}, R^{9″}, X″, Q″ and R^{11″} (where present) are as defined according to R², R⁶, R⁷, R⁹, X, Q and R¹¹ respectively, and R^{C} is a capping group.

Selected embodiments comprising the aforementioned structures are described in more detail immediately below.

Double Bond

In one embodiment, there is no double bond present between C1 and C2, and C2 and C3.

In one embodiment, the dotted lines indicate the optional presence of a double bond between C2 and C3, as shown below:

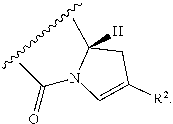

In one embodiment, a double bond is present between C2 and C3 when R² is C₅₋₂₀ aryl or C₁₋₁₂ alkyl. In a preferred embodiment R² comprises a methyl group.

In one embodiment, the dotted lines indicate the optional presence of a double bond between C1 and C2, as shown below:

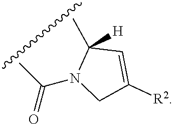

In one embodiment, a double bond is present between C1 and C2 when R² is C₅₋₂₀ aryl or C₁₋₁₂ alkyl. In a preferred embodiment R² comprises a methyl group.

R²

In one embodiment, R² is independently selected from H, OH, ═O, ═CH₂, CN, R, OR, ═CH—R^{D}, ═C(R^{D})₂, O—SO₂—R, CO₂R and COR, and optionally further selected from halo or dihalo.

In one embodiment, R² is independently selected from H, OH, ═O, ═CH₂, CN, R, OR, ═CH—R^{D}, ═C(R^{D})₂, O—SO₂-R, CO₂R and COR.

In one embodiment, R² is independently selected from H, ═O, ═CH₂, R, ═CH—R^{D}, and ═C(R^{D})₂.

In one embodiment, R² is independently H.

In one embodiment R² is independently R wherein R comprises CH₃.

In one embodiment, R² is independently ═O.

In one embodiment, R² is independently ═CH₂.

In one embodiment, R² is independently ═CH—R^{D}. Within the PBD compound, the group ═CH—R^{D} may have either configuration shown below:

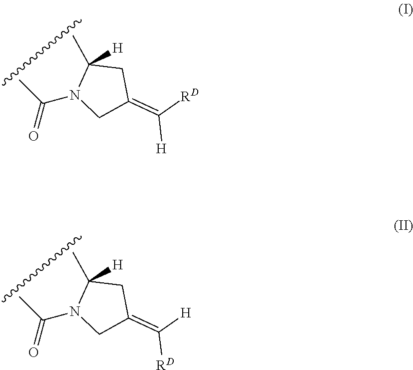

In one embodiment, the configuration is configuration (I).

In one embodiment, R² is independently ═C(R^{D})₂.

In one embodiment, R² is independently ═CF₂.

In one embodiment, R² is independently R.

In one embodiment, R² is independently optionally substituted C₅₋₂₀ aryl.

In one embodiment, R² is independently optionally substituted C₁₋₁₂ alkyl.

In one embodiment, R² is independently optionally substituted C₅₋₂₀ aryl.

In one embodiment, R² is independently optionally substituted C₅₋₇ aryl.

In one embodiment, R² is independently optionally substituted C₈₋₁₀ aryl.

In one embodiment, R² is independently optionally substituted phenyl.

In one embodiment, R² is independently optionally substituted napthyl.

In one embodiment, R² is independently optionally substituted pyridyl.

In one embodiment, R² is independently optionally substituted quinolinyl or isoquinolinyl.

In one embodiment, R² bears one to three substituent groups, with 1 and 2 being more preferred, and singly substituted groups being most preferred. The substituents may be any position.

Where R² is a C₅₋₇ aryl group, a single substituent is preferably on a ring atom that is not adjacent the bond to the remainder of the compound, i.e. it is preferably β or γ to the bond to the remainder of the compound. Therefore, where the C₅₋₇ aryl group is phenyl, the substituent is preferably in the meta- or para-positions, and more preferably is in the para-position.

In one embodiment, R² is selected from:

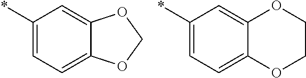

where the asterisk indicates the point of attachment.

Where R² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

In one embodiment, where R² is optionally substituted, the substituents are selected from those substituents given in the substituent section below.

Where R is optionally substituted, the substituents are preferably selected from:
Halo, Hydroxyl, Ether, Formyl, Acyl, Carboxy, Ester, Acyloxy, Amino, Amido, Acylamido, Aminocarbonyloxy, Ureido, Nitro, Cyano and Thioether.

In one embodiment, where R or R² is optionally substituted, the substituents are selected from the group consisting of R, OR, SR, NRR′, NO₂, halo, CO₂R, COR, CONH₂, CONHR, and CONRR′.

Where R² is C₁₋₁₂ alkyl, the optional substituent may additionally include C₃₋₂₀ heterocyclyl and C₅₋₂₀ aryl groups.

Where R² is C₃₋₂₀ heterocyclyl, the optional substituent may additionally include C₁₋₁₂ alkyl and C₅₋₂₀ aryl groups.

Where R² is C₅₋₂₀ aryl groups, the optional substituent may additionally include C₃₋₂₀ heterocyclyl and C₁₋₁₂ alkyl groups.

It is understood that the term “alkyl” encompasses the sub-classes alkenyl and alkynyl as well as cycloalkyl. Thus, where R² is optionally substituted C₁₋₁₂ alkyl, it is understood that the alkyl group optionally contains one or more carbon-carbon double or triple bonds, which may form part of a conjugated system. In one embodiment, the optionally substituted C₁₋₁₂ alkyl group contains at least one carbon-carbon double or triple bond, and this bond is conjugated with a double bond present between C1 and C2, or C2 and C3. In one embodiment, the C₁₋₁₂ alkyl group is a group selected from saturated C₁₋₁₂ alkyl, C₂₋₁₂ alkenyl, C₂₋₁₂ alkynyl and C₃₋₁₂ cycloalkyl.

If a substituent on R² is halo, it is preferably F or Cl, more preferably Cl.

If a substituent on R² is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy).

If a substituent on R² is C₁₋₇ alkyl, it may preferably be a C₁₋₄ alkyl group (e.g. methyl, ethyl, propyl, butyl).

If a substituent on R² is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups.

If a substituent on R² is bis-oxy-C₁₋₃ alkylene, this is preferably bis-oxy-methylene or bis-oxy-ethylene.

Particularly preferred substituents for R² include methoxy, ethoxy, fluoro, chloro, cyano, bis-oxy-methylene, methyl-piperazinyl, morpholino and methyl-thienyl.

Particularly preferred substituted R² groups include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthienyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl.

In one embodiment, R² is halo or dihalo. In one embodiment, R² is —F or —F₂, which substituents are illustrated below as (III) and (IV) respectively:

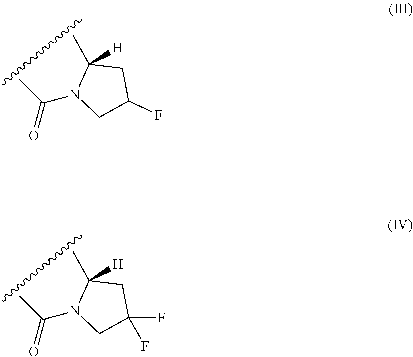

R^{D}

In one embodiment, R^{D} is independently selected from R, CO₂R, COR, CHO, CO₂H, and halo.

In one embodiment, R^{D} is independently R.

In one embodiment, R^{D} is independently halo.

R⁶

In one embodiment, R⁶ is independently selected from H, R, OH, OR, SH, SR, NH₂, NHR, NRR′, NO₂, Me₃Sn— and Halo.

In one embodiment, R⁶ is independently selected from H, OH, OR, SH, NH₂, NO₂ and Halo.

In one embodiment, R⁶ is independently selected from H and Halo.

In one embodiment, R⁶ is independently H.

In one embodiment, R⁶ and R⁷ together form a group —O—(CH₂)ₚ—O—, where p is 1 or 2.

R⁷

R⁷ is independently selected from H, R, OH, OR, SH, SR, NH₂, NHR, NRR′, NO₂, Me₃Sn and halo.

In one embodiment, R⁷ is independently OR.

In one embodiment, R⁷ is independently OR^{7A}, where R^{7A} is independently optionally substituted C₁₋₆ alkyl.

In one embodiment, R^{7A} is independently optionally substituted saturated C₁₋₆ alkyl.

In one embodiment, R^{7A} is independently optionally substituted C₂₋₄ alkenyl.

In one embodiment, R^{7A} is independently Me.

In one embodiment, R^{7A} is independently CH₂Ph.

In one embodiment, R^{7A} is independently allyl.

In one embodiment, the compound is a dimer where the R⁷ groups of each monomer form together a dimer bridge having the formula X—R″—X linking the monomers.

R⁹

In one embodiment, R⁹ is independently selected from H, R, OH, OR, SH, SR, NH₂, NHR, NRR′, NO₂, Me₃Sn— and Halo.

In one embodiment, R⁹ is independently H.

In one embodiment, R⁹ is independently R or OR.

R¹⁰

Preferably compatible linkers such as those described herein attach the CLDN antibody to the PBD drug moiety through covalent bond(s) at the R¹⁰ position (i.e., N10).

Q

In certain embodiments Q is independently selected from O, S and NH.

In one embodiment, Q is independently O.

In one embodiment, Q is independently S.

In one embodiment, Q is independently NH.

R¹¹

In selected embodiments R¹¹ is either H, or R or, where Q is O, may be SO₃M where M is a metal cation. The cation may be Na⁺.

In certain embodiments R¹¹ is H.

In certain embodiments R¹¹ is R.

In certain embodiments, where Q is O, R¹¹ is SO₃M where M is a metal cation. The cation may be Nat.

In certain embodiments where Q is O, R¹¹ is H.

In certain embodiments where Q is O, R¹¹ is R.

X

In one embodiment, X is selected from O, S, or N(H).

Preferably, X is O.

R″

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted.

In one embodiment, R″ is a C₃₋₁₂ alkylene group, which chain may be interrupted by one or more heteroatoms and/or aromatic rings, e.g. benzene or pyridine.

In one embodiment, the alkylene group is optionally interrupted by one or more heteroatoms selected from O, S, and NMe and/or aromatic rings, which rings are optionally substituted.

In one embodiment, the aromatic ring is a C₅₋₂₀ arylene group, where arylene pertains to a divalent moiety obtained by removing two hydrogen atoms from two aromatic ring atoms of an aromatic compound, which moiety has from 5 to 20 ring atoms.

In one embodiment, R″ is a C₃₋₁₂ alkylene group, which chain may be interrupted by one or more heteroatoms, e.g. O, S, N(H), NMe and/or aromatic rings, e.g. benzene or pyridine, which rings are optionally substituted by NH₂.

In one embodiment, R″ is a C₃₋₁₂ alkylene group.

In one embodiment, R″ is selected from a C₃, C₅, C₇, C₉ and a C₁₁ alkylene group.

In one embodiment, R″ is selected from a C₃, C₅ and a C₇ alkylene group.

In one embodiment, R″ is selected from a C₃ and a C₅ alkylene group.

In one embodiment, R″ is a C₃ alkylene group.

In one embodiment, R″ is a C₅ alkylene group.

The alkylene groups listed above may be optionally interrupted by one or more heteroatoms and/or aromatic rings, e.g. benzene or pyridine, which rings are optionally substituted.

The alkylene groups listed above may be optionally interrupted by one or more heteroatoms and/or aromatic rings, e.g. benzene or pyridine.

The alkylene groups listed above may be unsubstituted linear aliphatic alkylene groups.

R and R′

In one embodiment, R is independently selected from optionally substituted C₁₋₁₂ alkyl, C₃₋₂₀ heterocyclyl and C₅₋₂₀ aryl groups.

In one embodiment, R is independently optionally substituted C₁₋₁₂ alkyl.

In one embodiment, R is independently optionally substituted C₃₋₂₀ heterocyclyl.

In one embodiment, R is independently optionally substituted C₅₋₂₀ aryl.

Described above in relation to R² are various embodiments relating to preferred alkyl and aryl groups and the identity and number of optional substituents. The preferences set out for R² as it applies to R are applicable, where appropriate, to all other groups R, for examples where R⁶, R⁷, R⁸ or R⁹ is R.

The preferences for R apply also to R′.

In some embodiments of the invention there is provided a compound having a substituent group —NRR′. In one embodiment, R and R′ together with the nitrogen atom to which they are attached form an optionally substituted 4-, 5-, 6- or 7-membered heterocyclic ring. The ring may contain a further heteroatom, for example N, O or S.

In one embodiment, the heterocyclic ring is itself substituted with a group R. Where a further N heteroatom is present, the substituent may be on the N heteroatom.

In addition to the aforementioned PBDs certain dimeric PBDs have been shown to be particularly active and may be used in conjunction with the instant invention. To this end antibody drug conjugates (i.e., ADCs 1-6 as disclosed herein) of the instant invention may comprise a PBD compound set forth immediately below as PBD 1-5. Note that PBDs 1-5 below comprise the cytotoxic warhead released following separation of a linker such as those described in more detail herein. The synthesis of each of PBD 1-5 as a component of drug-linker compounds is presented in great detail in WO 2014/130879 which is hereby incorporated by reference as to such synthesis. In view of WO 2014/130879 cytotoxic compounds that may comprise selected warheads of the ADCs of the present invention could readily be generated and employed as set forth herein. Accordingly, selected PBD compounds that may be released from the disclosed ADCs upon separation from a linker are set forth immediately below:

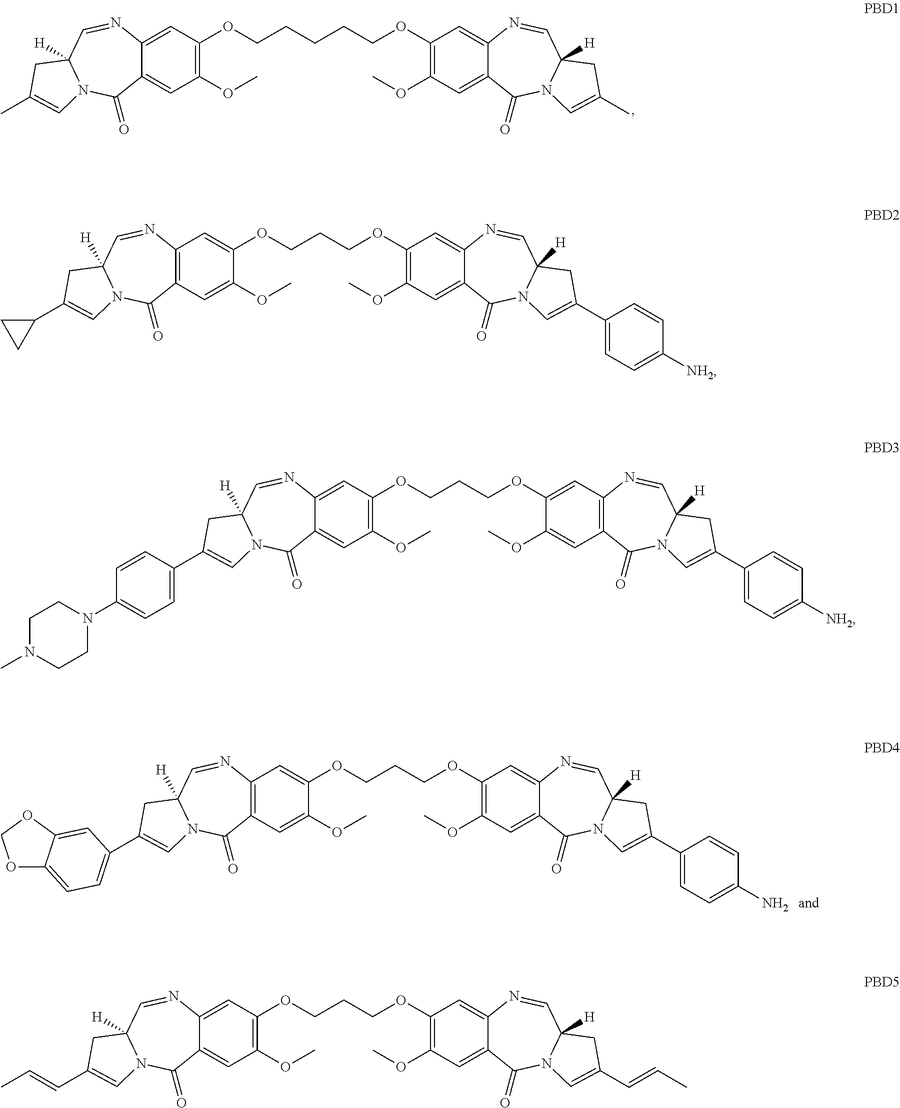

It will be appreciated that each of the aforementioned dimeric PBD warheads will preferably be released upon internalization by the target cell and destruction of the linker. As described in more detail below, certain linkers will comprise cleavable linkers which may incorporate a self-immolation moiety that allows release of the active PBD warhead without retention of any part of the linker. Upon release the PBD warhead will then bind and cross-link with the target cell's DNA. Such binding reportedly blocks division of the target cancer cell without distorting its DNA helix, thus potentially avoiding the common phenomenon of emergent drug resistance. In other preferred embodiments the warhead may be attached to the CLDN targeting moiety through a cleavable linker that does not comprise a self-immolating moiety.

Delivery and release of such compounds at the tumor site(s) may prove clinically effective in treating or managing proliferative disorders in accordance with the instant disclosure. With regard to the compounds it will be appreciated that each of the disclosed PBDs have two sp² centers in each C-ring, which may allow for stronger binding in the minor groove of DNA (and hence greater toxicity), than for compounds with only one sp² center in each C-ring. Thus, when used in CLDN ADCs as set forth herein the disclosed PBDs may prove to be particularly effective for the treatment of proliferative disorders.

The foregoing provides exemplary PBD compounds that are compatible with the instant invention and is in no way meant to be limiting as to other PBDs that may be successfully incorporated in anti-CLDN conjugates according to the teachings herein. Rather, any PBD that may be conjugated to an antibody as described herein and set forth in the Examples below is compatible with the disclosed conjugates and expressly within the metes and bounds of the invention.

In addition to the aforementioned agents the antibodies of the present invention may also be conjugated to biological response modifiers. In certain embodiments the biological response modifier will comprise interleukin 2, interferons, or various types of colony-stimulating factors (e.g., CSF, GM-CSF, G-CSF).

More generally, the associated drug moiety can be a polypeptide possessing a desired biological activity. Such proteins may include, for example, a toxin such as abrin, ricin A, Onconase (or another cytotoxic RNase), pseudomonas exotoxin, cholera toxin, diphtheria toxin; an apoptotic agent such as tumor necrosis factor e.g. TNF-α or TNF-β, α-interferon, β-interferon, nerve growth factor, platelet derived growth factor, tissue plasminogen activator, AIM I (WO 97/33899), AIM II (WO 97/34911), Fas Ligand (Takahashi et al., 1994, PMID: 7826947), and VEGI (WO 99/23105), a thrombotic agent, an anti-angiogenic agent, e.g., angiostatin or endostatin, a lymphokine, for example, interleukin-1 (IL-1), interleukin-2 (IL-2), interleukin-6 (IL-6), granulocyte macrophage colony stimulating factor (GM-CSF), and granulocyte colony stimulating factor (G-CSF), or a growth factor e.g., growth hormone (GH).

2. Diagnostic or Detection Agents

In other embodiments, the antibodies of the invention, or fragments or derivatives thereof, are conjugated to a diagnostic or detectable agent, marker or reporter which may be, for example, a biological molecule (e.g., a peptide or nucleotide), a small molecule, fluorophore, or radioisotope. Labeled antibodies can be useful for monitoring the development or progression of a hyperproliferative disorder or as part of a clinical testing procedure to determine the efficacy of a particular therapy including the disclosed antibodies (i.e. theragnostics) or to determine a future course of treatment. Such markers or reporters may also be useful in purifying the selected antibody, for use in antibody analytics (e.g., epitope binding or antibody binning), separating or isolating tumorigenic cells or in preclinical procedures or toxicology studies.

Such diagnosis, analysis and/or detection can be accomplished by coupling the antibody to detectable substances including, but not limited to, various enzymes comprising for example horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I), carbon (¹⁴C), sulfur (³⁵S), tritium (³H), indium (¹¹⁵In, ¹¹³In, ¹¹²In, ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ⁸⁹Zr, ¹⁵³Gd, ¹⁶⁹Yb, ⁵¹Cr, ⁵⁴Mn, ⁷⁵Se, ¹¹³Sn, and ¹¹⁷Tin; positron emitting metals using various positron emission tomographies, non-radioactive paramagnetic metal ions, and molecules that are radiolabeled or conjugated to specific radioisotopes. In such embodiments appropriate detection methodology is well known in the art and readily available from numerous commercial sources.

In other embodiments the antibodies or fragments thereof can be fused or conjugated to marker sequences or compounds, such as a peptide or fluorophore to facilitate purification or diagnostic or analytic procedures such as immunohistochemistry, bio-layer interferometry, surface plasmon resonance, flow cytometry, competitive ELISA, FACs, etc. In some embodiments, the marker comprises a histidine tag such as that provided by the pQE vector (Qiagen), among others, many of which are commercially available. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag (U.S. Pat. No. 4,703,004).

3. Biocompatible Modifiers

In selected embodiments the antibodies of the invention may be conjugated with biocompatible modifiers that may be used to adjust, alter, improve or moderate antibody characteristics as desired. For example, antibodies or fusion constructs with increased in vivo half-lives can be generated by attaching relatively high molecular weight polymer molecules such as commercially available polyethylene glycol (PEG) or similar biocompatible polymers. Those skilled in the art will appreciate that PEG may be obtained in many different molecular weights and molecular configurations that can be selected to impart specific properties to the antibody (e.g. the half-life may be tailored). PEG can be attached to antibodies or antibody fragments or derivatives with or without a multifunctional linker either through conjugation of the PEG to the N- or C-terminus of said antibodies or antibody fragments or via epsilon-amino groups present on lysine residues. Linear or branched polymer derivatization that results in minimal loss of biological activity may be used. The degree of conjugation can be closely monitored by SDS-PAGE and mass spectrometry to ensure optimal conjugation of PEG molecules to antibody molecules. Unreacted PEG can be separated from antibody-PEG conjugates by, e.g., size exclusion or ion-exchange chromatography. In a similar manner, the disclosed antibodies can be conjugated to albumin in order to make the antibody or antibody fragment more stable in vivo or have a longer half-life in vivo. The techniques are well known in the art, see e.g., WO 93/15199, WO 93/15200, and WO 01/77137; and EP 0 413, 622. Other biocompatible conjugates are evident to those of ordinary skill and may readily be identified in accordance with the teachings herein.

B. Linker Compounds

As indicated above payloads compatible with the instant invention comprise one or more warheads and, optionally, a linker associating the warheads with the antibody targeting agent. Numerous linker compounds can be used to conjugate the antibodies of the invention to the relevant warhead. The linkers merely need to covalently bind with the reactive residue on the antibody (preferably a cysteine or lysine) and the selected drug compound. Accordingly, any linker that reacts with the selected antibody residue and may be used to provide the relatively stable conjugates (site-specific or otherwise) of the instant invention is compatible with the teachings herein.

Compatible linkers can advantageously bind to reduced cysteines and lysines, which are nucleophilic. Conjugation reactions involving reduced cysteines and lysines include, but are not limited to, thiol-maleimide, thiol-halogeno (acyl halide), thiol-ene, thiol-yne, thiol-vinylsulfone, thiol-bisulfone, thiol-thiosulfonate, thiol-pyridyl disulfide and thiol-parafluoro reactions. As further discussed herein, thiol-maleimide bioconjugation is one of the most widely used approaches due to its fast reaction rates and mild conjugation conditions. One issue with this approach is the possibility of the retro-Michael reaction and loss or transfer of the maleimido-linked payload from the antibody to other proteins in the plasma, such as, for example, human serum albumin. However, in some embodiments the use of selective reduction and site-specific antibodies as set forth herein in the Examples below may be used to stabilize the conjugate and reduce this undesired transfer. Thiol-acyl halide reactions provide bioconjugates that cannot undergo retro-Michael reaction and therefore are more stable. However, the thiol-halide reactions in general have slower reaction rates compared to maleimide-based conjugations and are thus not as efficient in providing undesired drug to antibody ratios. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulfone and thiol-bisulfone reactions, each of which are compatible with the teachings herein and expressly included within the scope of the invention.

In selected embodiments compatible linkers will confer stability on the ADCs in the extracellular environment, prevent aggregation of the ADC molecules and keep the ADC freely soluble in aqueous media and in a monomeric state. Before transport or delivery into a cell, the ADC is preferably stable and remains intact, i.e. the antibody remains linked to the drug moiety. While the linkers are stable outside the target cell they may be designed to be cleaved or degraded at some efficacious rate inside the cell. Accordingly an effective linker will: (i) maintain the specific binding properties of the antibody; (ii) allow intracellular delivery of the conjugate or drug moiety; (iii) remain stable and intact, i.e. not cleaved or degraded, until the conjugate has been delivered or transported to its targeted site; and (iv) maintain a cytotoxic, cell-killing effect or a cytostatic effect of the drug moiety (including, in some cases, any bystander effects). The stability of the ADC may be measured by standard analytical techniques such as HPLC/UPLC, mass spectroscopy, HPLC, and the separation/analysis techniques LC/MS and LC/MS/MS. As set forth above covalent attachment of the antibody and the drug moiety requires the linker to have two reactive functional groups, i.e. bivalency in a reactive sense. Bivalent linker reagents that are useful to attach two or more functional or biologically active moieties, such as MMAE and antibodies are known, and methods have been described to provide resulting conjugates compatible with the teachings herein.

Linkers compatible with the present invention may broadly be classified as cleavable and non-cleavable linkers. Cleavable linkers, which may include acid-labile linkers (e.g., oximes and hydrozones), protease cleavable linkers and disulfide linkers, are internalized into the target cell and are cleaved in the endosomal-lysosomal pathway inside the cell. Release and activation of the cytotoxin relies on endosome/lysosome acidic compartments that facilitate cleavage of acid-labile chemical linkages such as hydrazone or oxime. If a lysosomal-specific protease cleavage site is engineered into the linker the cytotoxins will be released in proximity to their intracellular targets. Alternatively, linkers containing mixed disulfides provide an approach by which cytotoxic payloads are released intracellularly as they are selectively cleaved in the reducing environment of the cell, but not in the oxygen-rich environment in the bloodstream. By way of contrast, compatible non-cleavable linkers containing amide linked polyethylene glycol or alkyl spacers liberate toxic payloads during lysosomal degradation of the ADC within the target cell. In some respects the selection of linker will depend on the particular drug used in the conjugate, the particular indication and the antibody target.

Accordingly, certain embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, each of which is known to hydrolyze dipeptide drug derivatives resulting in the release of active drug inside target cells. Exemplary peptidyl linkers that are cleavable by the thiol-dependent protease cathepsin-B are peptides comprising Phe-Leu since cathepsin-B has been found to be highly expressed in cancerous tissue. Other examples of such linkers are described, for example, in U.S. Pat. No. 6,214,345. In specific embodiments, the peptidyl linker cleavable by an intracellular protease is a Val-Cit linker, a Val-Ala linker or a Phe-Lys linker. One advantage of using intracellular proteolytic release of the therapeutic agent is that the agent is typically attenuated when conjugated and the serum stabilities of the conjugates are relatively high.

In other embodiments, the cleavable linker is pH-sensitive. Typically, the pH-sensitive linker will be hydrolyzable under acidic conditions. For example, an acid-labile linker that is hydrolyzable in the lysosome (e.g., a hydrazone, oxime, semicarbazone, thiosemicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable (e.g., cleavable) at below pH 5.5 or 5.0 which is the approximate pH of the lysosome.

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio)toluene). In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In certain aspects of the invention the selected linker will comprise a compound of the formula:

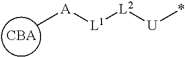

wherein the asterisk indicates the point of attachment to the drug, CBA (i.e. cell binding agent) comprises the anti-CLDN antibody, L¹ comprises a linker unit and optionally a cleavable linker unit, A is a connecting group (optionally comprising a spacer) connecting L¹ to a reactive residue on the antibody, L² is preferably a covalent bond and U, which may or may not be present, can comprise all or part of a self-immolative unit that facilitates a clean separation of the linker from the warhead at the tumor site.

In some embodiments (such as those set forth in U.S.P.N. 2011/0256157) compatible linkers may comprise:

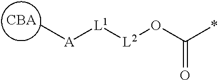

where the asterisk indicates the point of attachment to the drug, CBA (i.e. cell binding agent) comprises the anti-CLDN antibody, L¹ comprises a linker and optionally a cleavable linker, A is a connecting group (optionally comprising a spacer) connecting L¹ to a reactive residue on the antibody and L² is a covalent bond or together with —OC(═O)— forms a self-immolative moiety.

It will be appreciated that the nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidizing conditions may also find use in the present invention.

In certain embodiments L¹ may comprise a contiguous sequence of amino acids. The amino acid sequence may be the target substrate for enzymatic cleavage, thereby allowing release of the drug.

In one embodiment, L¹ is cleavable by the action of an enzyme. In one embodiment, the enzyme is an esterase or a peptidase.

In another embodiment L¹ is as a cathepsin labile linker.

In one embodiment, L¹ comprises a dipeptide. The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from: -Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit- where Cit is citrulline.

Preferably, the group —X₁—X₂— in dipeptide, —NH—X₁—X₂—CO—, is selected from: -Phe-Lys-, -Val-Ala-, -Val-Lys-, -Ala-Lys-, and -Val-Cit-.

Most preferably, the group —X₁—X₂— in dipeptide, —NH—X₁—X₂—CO—, is -Phe-Lys- or -Val-Ala- or Val-Cit. In certain selected embodiments the dipeptide will comprise -Val-Ala-.

In one embodiment, L² is present in the form of a covalent bond.

In one embodiment, L² is present and together with —C(═O)O— forms a self-immolative linker.

In one embodiment, L² is a substrate for enzymatic activity, thereby allowing release of the warhead.

In one embodiment, where L¹ is cleavable by the action of an enzyme and L² is present, the enzyme cleaves the bond between L¹ and L².

L¹ and L², where present, may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴O, ¹⁵N), protected forms, and racemic mixtures thereof.

In one embodiment, —C(═O)O— and L² together form the group:

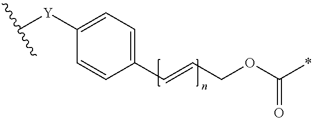

where the asterisk indicates the point of attachment to the drug or cytotoxic agent position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents. In one embodiment, the phenylene group is optionally substituted with halo, NO₂, alkyl or hydroxyalkyl.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In other embodiments the linker may include a self-immolative linker and the dipeptide together form the group —NH-Val-Cit-CO—NH-PABC-. In other selected embodiments the linker may comprise the group —NH-Val-Ala-CO—NH-PABC-, which is illustrated below:

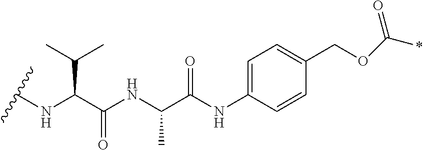 [SMILES: *N[C@H](C(=O)N[C@@H](C)C(=O)Nc1ccc(COC(*)=O)cc1)C(C)C]

where the asterisk indicates the point of attachment to the selected cytotoxic moiety, and the wavy line indicates the point of attachment to the remaining portion of the linker (e.g., the spacer-antibody binding segments) which may be conjugated to the antibody. Upon enzymatic cleavage of the dipeptide, the self-immolative linker will allow for clean release of the protected compound (i.e., the cytotoxin) when a remote site is activated, proceeding along the lines shown below:

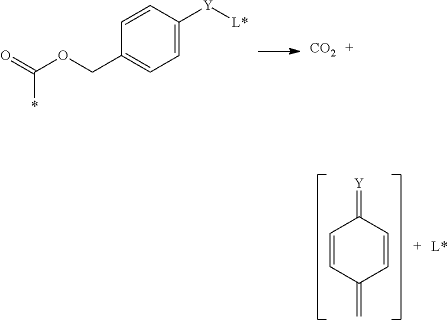

where the asterisk indicates the point of attachment to the selected cytotoxic moiety and where L* is the activated form of the remaining portion of the linker comprising the now cleaved peptidyl unit. The clean release of the warhead ensures it will maintain the desired toxic activity.

In one embodiment, A is a covalent bond. Thus, L¹ and the antibody are directly connected. For example, where L¹ comprises a contiguous amino acid sequence, the N-terminus of the sequence may connect directly to the antibody residue.

In another embodiment, A is a spacer group. Thus, L¹ and the antibody are indirectly connected.

In certain embodiments L¹ and A may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

As will be discussed in more detail below the drug linkers of the instant invention will preferably be linked to reactive thiol nucleophiles on cysteines, including free cysteines. To this end the cysteines of the antibodies may be made reactive for conjugation with linker reagents by treatment with various reducing agent such as DTT or TCEP or mild reducing agents as set forth herein. In other embodiments the drug linkers of the instant invention will preferably be linked to a lysine.

Preferably, the linker contains an electrophilic functional group for reaction with a nucleophilic functional group on the antibody. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones and carboxyl groups.

Exemplary functional groups compatible with the invention are illustrated immediately below:

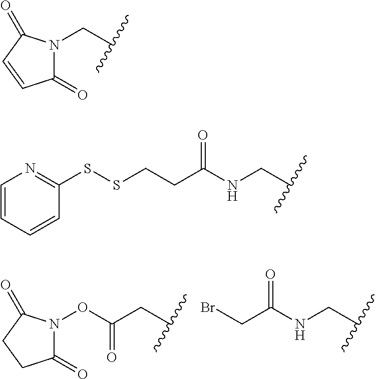

In some embodiments the connection between a cysteine (including a free cysteine of a site-specific antibody) and the drug-linker moiety is through a thiol residue and a terminal maleimide group of present on the linker. In such embodiments, the connection between the antibody and the drug-linker may be:

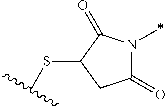 [SMILES: *SC1CC(=O)N(*)C1=O]

where the asterisk indicates the point of attachment to the remaining portion of drug-linker and the wavy line indicates the point of attachment to the remaining portion of the antibody. In such embodiments, the S atom may preferably be derived from a site-specific free cysteine.

With regard to other compatible linkers the binding moiety may comprise a terminal bromo or iodoacetamide that may be reacted with activated residues on the antibody to provide the desired conjugate. In any event one skilled in the art could readily conjugate each of the disclosed drug-linker compounds with a compatible anti-CLDN antibody (including site-specific antibodies) in view of the instant disclosure.

In accordance with the instant disclosure the invention provides methods of making compatible antibody drug conjugates comprising conjugating an anti-CLDN antibody with a drug-linker compound selected from the group consisting of:

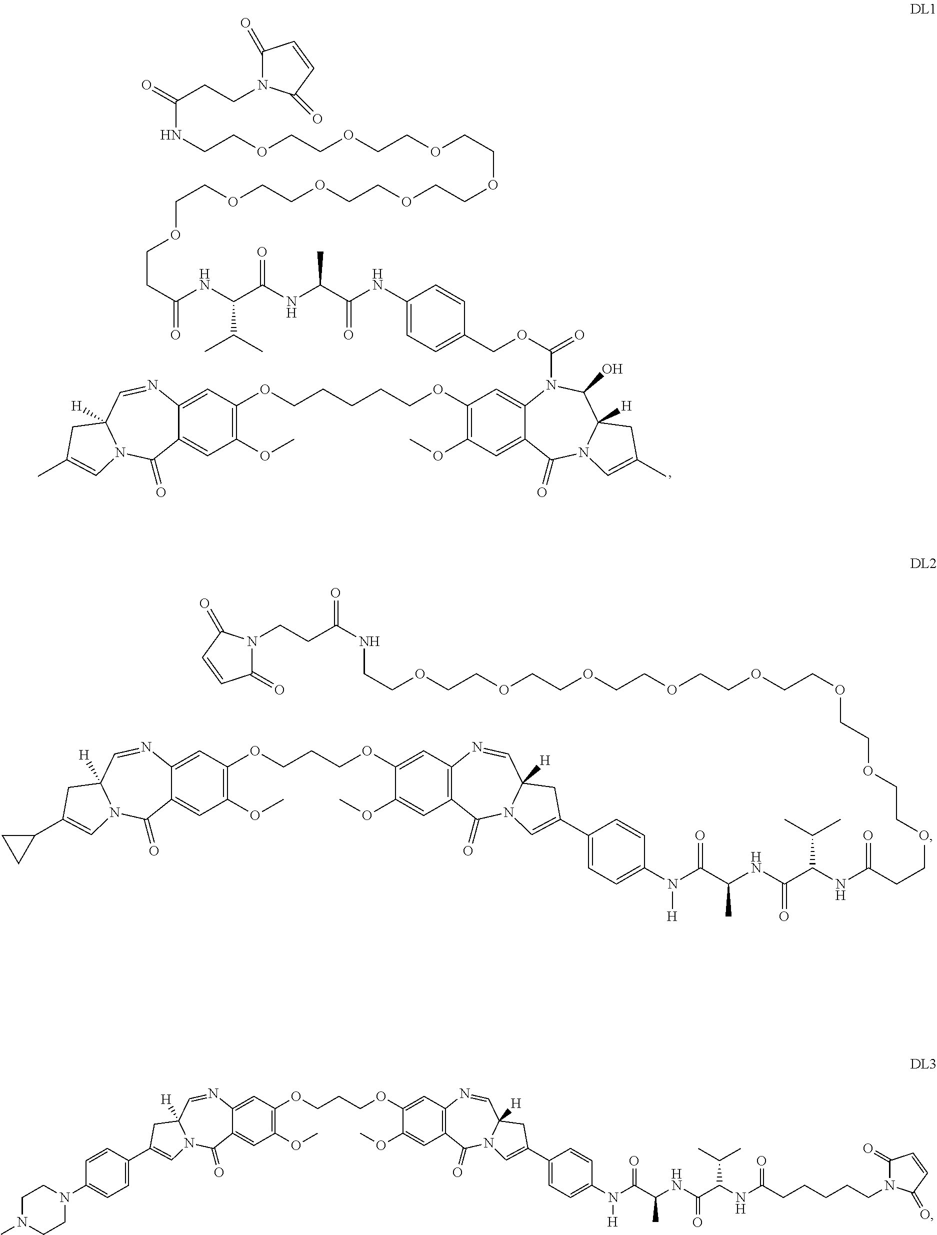

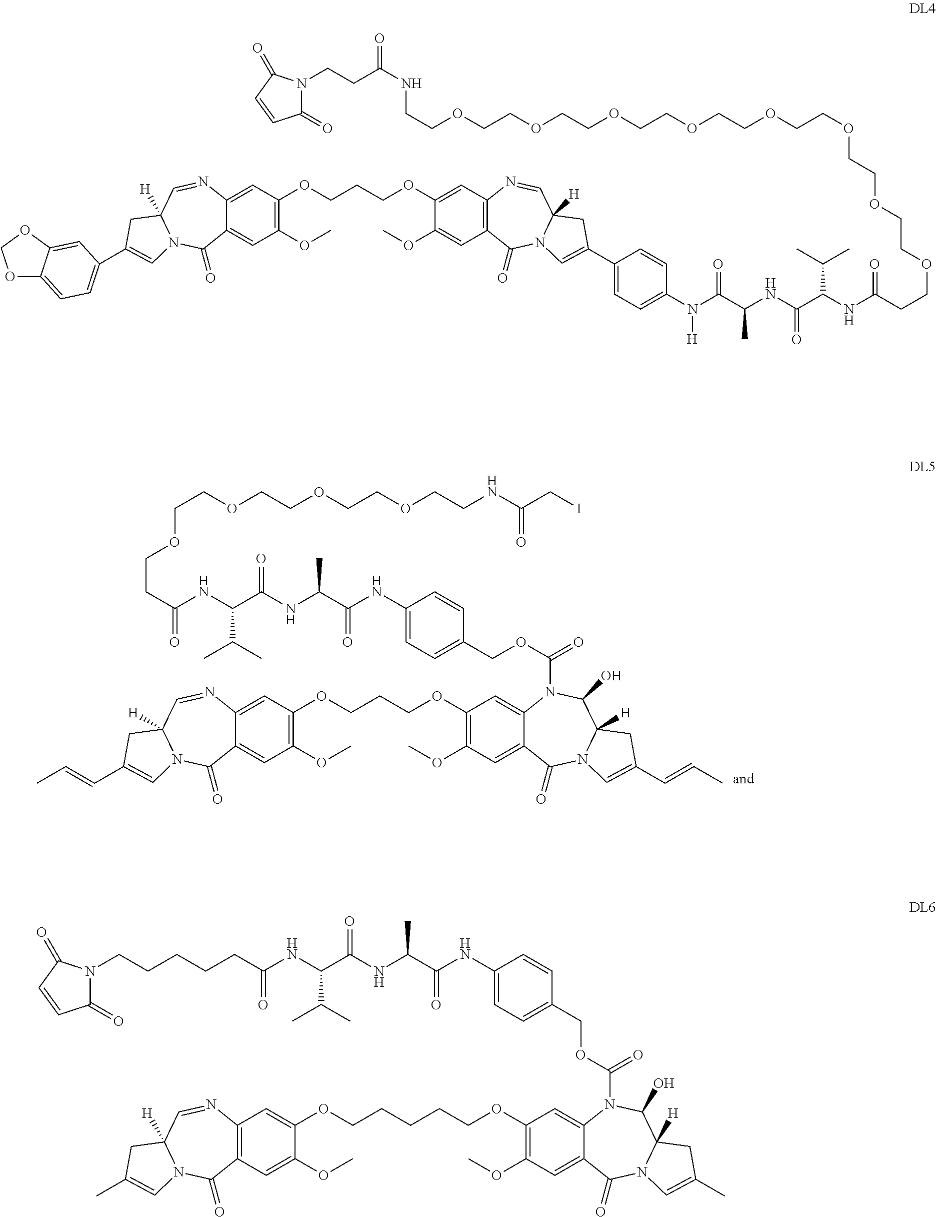

For the purposes of then instant application DL will be used as an abbreviation for “drug-linker” and will comprise drug linkers 1-6 (i.e., DL1, DL2, DL3, DL4 DL5, and DL6) as set forth above. Note that DL1 and DL6 comprise the same warhead and same dipeptide subunit but differ in the connecting group spacer. Accordingly, upon cleavage of the linker both DL1 and DL6 will release PBD1.

It will be appreciated that the linker appended terminal maleimido moiety (DL1-DL4 and DL6) or iodoacetamide moiety (DL5) may be conjugated to free sulfhydryl(s) on the selected CLDN antibody using art-recognized techniques. Synthetic routes for the aforementioned compounds are set forth in WO2014/130879 which is incorporated herein by reference explicitly for the synthesis of the aforementioned DL compounds while specific methods of conjugating such PBDs linker combinations are set forth in the Examples below.

Thus, in selected aspects the present invention relates to CLDN antibodies conjugated to the disclosed DL moieties to provide CLDN immunoconjugates substantially set forth in ADCs 1-6 immediately below. Accordingly, in certain aspects the invention is directed to an antibody drug conjugate selected from the group consisting of

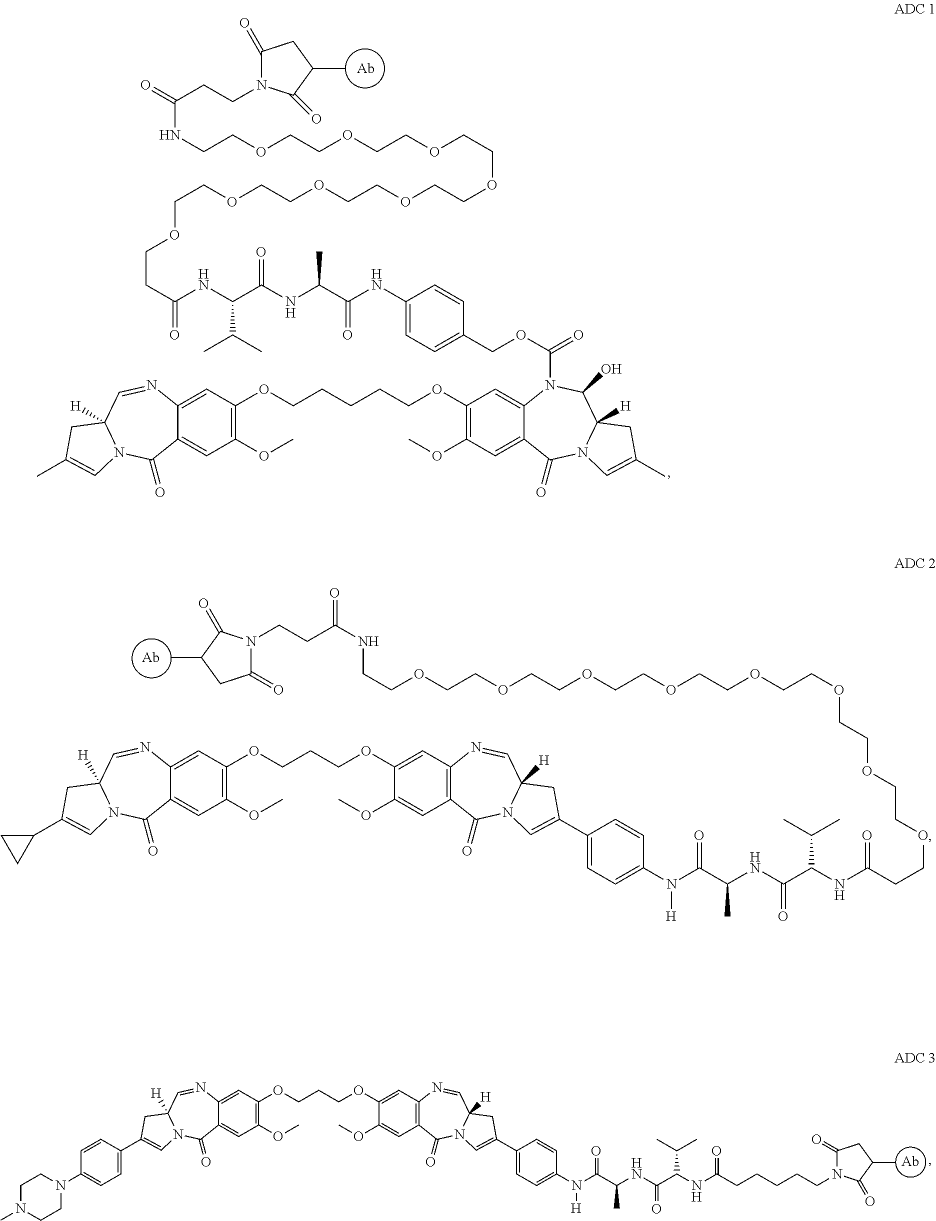

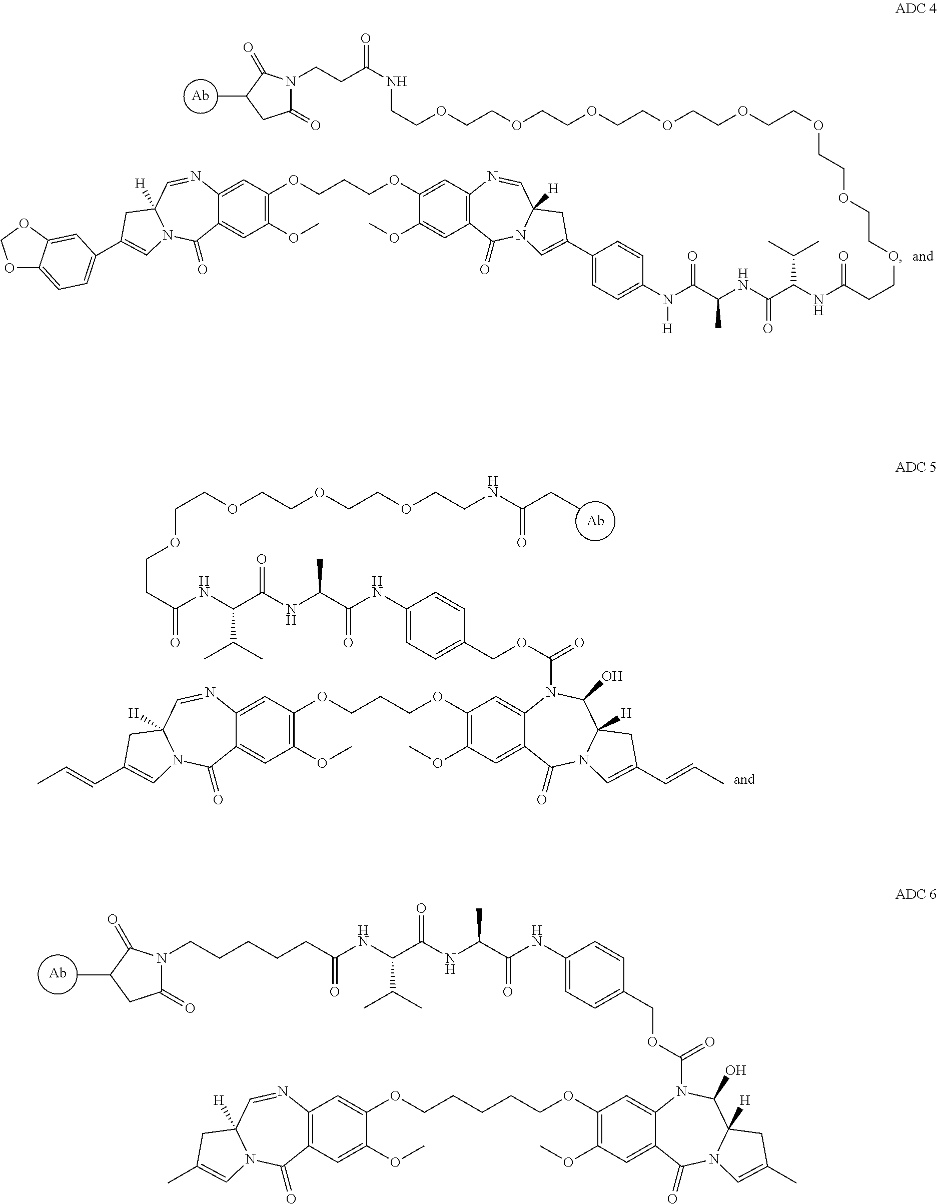

wherein Ab comprises an anti-CLDN antibody or immunoreactive fragment thereof.

In certain aspects the CLDN PBD ADCs of the invention will comprise an anti-CLDN antibody as set forth in the appended Examples or an immunoreactive fragment thereof. In a particular embodiment ADC 3 will comprise hSC27.204v2ss1 (e.g., hSC27.204v2ss1 PBD3). In other aspects the CLDN PBD ADCs of the invention will comprise ADC 1 or ADC 6 incorporating the cell binding agent hSC27.204v2ss1 (e.g., hSC27.204v2ss1 PBD1).

C. Conjugation

It will be appreciated that a number of well-known reactions may be used to attach the drug moiety and/or linker to the selected antibody. For example, various reactions exploiting sulfhydryl groups of cysteines may be employed to conjugate the desired moiety. Some embodiments will comprise conjugation of antibodies comprising one or more free cysteines as discussed in detail below. In other embodiments ADCs of the instant invention may be generated through conjugation of drugs to solvent-exposed amino groups of lysine residues present in the selected antibody. Still other embodiments comprise activation of N-terminal threonine and serine residues which may then be used to attach the disclosed payloads to the antibody. The selected conjugation methodology will preferably be tailored to optimize the number of drugs attached to the antibody and provide a relatively high therapeutic index.

Various methods are known in the art for conjugating a therapeutic compound to a cysteine residue and will be apparent to the skilled artisan. Under basic conditions the cysteine residues will be deprotonated to generate a thiolate nucleophile which may be reacted with soft electrophiles such as maleimides and iodoacetamides. Generally reagents for such conjugations may react directly with a cysteine thiol to form the conjugated protein or with a linker-drug to form a linker-drug intermediate. In the case of a linker, several routes, employing organic chemistry reactions, conditions, and reagents are known to those skilled in the art, including: (1) reaction of a cysteine group of the protein of the invention with a linker reagent, to form a protein-linker intermediate, via a covalent bond, followed by reaction with an activated compound; and (2) reaction of a nucleophilic group of a compound with a linker reagent, to form a drug-linker intermediate, via a covalent bond, followed by reaction with a cysteine group of a protein of the invention. As will be apparent to the skilled artisan from the foregoing, bifunctional (or bivalent) linkers are useful in the present invention. For example, the bifunctional linker may comprise a thiol modification group for covalent linkage to the cysteine residue(s) and at least one attachment moiety (e.g., a second thiol modification moiety) for covalent or non-covalent linkage to the compound.

Prior to conjugation, antibodies may be made reactive for conjugation with linker reagents by treatment with a reducing agent such as dithiothreitol (DTT) or (tris(2-carboxyethyl)phosphine (TCEP). In other embodiments additional nucleophilic groups can be introduced into antibodies through the reaction of lysines with reagents, including but not limited to, 2-iminothiolane (Traut's reagent), SATA, SATP or SAT(PEG)4, resulting in conversion of an amine into a thiol.

With regard to such conjugations cysteine thiol or lysine amino groups are nucleophilic and capable of reacting to form covalent bonds with electrophilic groups on linker reagents or compound-linker intermediates or drugs including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a compound or linker include, but are not limited to amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Conjugation reagents commonly include maleimide, haloacetyl, iodoacetamide succinimidyl ester, isothiocyanate, sulfonyl chloride, 2,6-dichlorotriazinyl, pentafluorophenyl ester, and phosphoramidite, although other functional groups can also be used. In certain embodiments methods include, for example, the use of maleimides, iodoacetimides or haloacetyl/alkyl halides, aziridne, acryloyl derivatives to react with the thiol of a cysteine to produce a thioether that is reactive with a compound. Disulphide exchange of a free thiol with an activated piridyldisulphide is also useful for producing a conjugate (e.g., use of 5-thio-2-nitrobenzoic (TNB) acid). Preferably, a maleimide is used.

As indicated above, lysine may also be used as a reactive residue to effect conjugation as set forth herein. The nucleophilic lysine residue is commonly targeted through amine-reactive succinimidylesters. To obtain an optimal number of deprotonated lysine residues, the pH of the aqueous solution must be below the pKa of the lysine ammonium group, which is around 10.5, so the typical pH of the reaction is about 8 and 9. The common reagent for the coupling reaction is NHS-ester which reacts with nucleophilic lysine through a lysine acylation mechanism. Other compatible reagents that undergo similar reactions comprise isocyanates and isothiocyanates which also may be used in conjunction with the teachings herein to provide ADCs. Once the lysines have been activated, many of the aforementioned linking groups may be used to covalently bind the warhead to the antibody.

Methods are also known in the art for conjugating a compound to a threonine or serine residue (preferably a N-terminal residue). For example methods have been described in which carbonyl precursors are derived from the 1,2-aminoalcohols of serine or threonine, which can be selectively and rapidly converted to aldehyde form by periodate oxidation. Reaction of the aldehyde with a 1,2-aminothiol of cysteine in a compound to be attached to a protein of the invention forms a stable thiazolidine product. This method is particularly useful for labeling proteins at N-terminal serine or threonine residues.

In some embodiments reactive thiol groups may be introduced into the selected antibody (or fragment thereof) by introducing one, two, three, four, or more free cysteine residues (e.g., preparing antibodies comprising one or more free non-native cysteine amino acid residues). Such site-specific antibodies or engineered antibodies allow for conjugate preparations that exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites (and is fully compatible with the instant invention), the present invention additionally provides for the selective reduction of certain prepared free cysteine sites and attachment of the drug-linker to the same.

In this regard it will be appreciated that the conjugation specificity promoted by the engineered sites and the selective reduction allows for a high percentage of site directed conjugation at the desired positions. Significantly some of these conjugation sites, such as those present in the terminal region of the light chain constant region, are typically difficult to conjugate effectively as they tend to cross-react with other free cysteines. However, through molecular engineering and selective reduction of the resulting free cysteines, efficient conjugation rates may be obtained which considerably reduces unwanted high-DAR contaminants and non-specific toxicity. More generally the engineered constructs and disclosed novel conjugation methods comprising selective reduction provide ADC preparations having improved pharmacokinetics and/or pharmacodynamics and, potentially, an improved therapeutic index.

In certain embodiments site-specific constructs present free cysteine(s) which, when reduced, comprise thiol groups that are nucleophilic and capable of reacting to form covalent bonds with electrophilic groups on linker moieties such as those disclosed above. As discussed above antibodies of the instant invention may have reducible unpaired interchain or intrachain cysteines or introduced non-native cysteines, i.e. cysteines providing such nucleophilic groups. Thus, in certain embodiments the reaction of free sulfhydryl groups of the reduced free cysteines and the terminal maleimido or haloacetamide groups of the disclosed drug-linkers will provide the desired conjugation. In such cases free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are particularly compatible with the instant invention it will be appreciated that conjugation of site-specific antibodies may be effected using various reactions, conditions and reagents generally known to those skilled in the art.

In addition it has been found that the free cysteines of engineered antibodies may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein. As used herein the terms “selective reduction” or “selectively reducing” may be used interchangeably and shall mean the reduction of free cysteine(s) without substantially disrupting native disulfide bonds present in the engineered antibody. In selected embodiments this selective reduction may be effected by the use of certain reducing agents or certain reducing agent concentrations. In other embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced in the presence of a cytotoxin as described herein. In this respect the use of such stabilizing agents (e.g., arginine) in combination with selected reducing agents can markedly improve the efficiency of site-specific conjugation as determined by extent of conjugation on the heavy and light antibody chains and DAR distribution of the preparation. Compatible antibody constructs and selective conjugation techniques and reagents are extensively disclosed in WO2015/031698 which is incorporated herein specifically as to such methodology and constructs.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site(s). Such agents (e.g., certain amino acids) are known to form salt bridges (via hydrogen bonding and electrostatic interactions) and can modulate protein-protein interactions in such a way as to impart a stabilizing effect that may cause favorable conformational changes and/or reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the drug (preferably via a linker). Since selective reduction conditions do not provide for the significant reduction of intact native disulfide bonds, the subsequent conjugation reaction is naturally driven to the relatively few reactive thiols on the free cysteines (e.g., preferably 2 free thiols per antibody). As previously alluded to, such techniques may be used to considerably reduce levels of non-specific conjugation and corresponding unwanted DAR species in conjugate preparations fabricated in accordance with the instant disclosure.

In selected embodiments stabilizing agents compatible with the present invention will generally comprise compounds with at least one moiety having a basic pKa. In certain embodiments the moiety will comprise a primary amine while in other embodiments the amine moiety will comprise a secondary amine. In still other embodiments the amine moiety will comprise a tertiary amine or a guanidinium group. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In some embodiments, compatible stabilizing agents may comprise arginine, lysine, proline and cysteine. In certain preferred embodiments the stabilizing agent will comprise arginine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other embodiments will comprise stabilizing agents where the amine moiety will have a pKa of greater than about 9.5 while certain other embodiments will comprise stabilizing agents exhibiting at least one amine moiety having a pKa of greater than about 10.0. In still other embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the native disulfide bonds of the engineered antibody. Under such conditions, preferably provided by the combination of selected stabilizing and reducing agents, the activated drug linker is largely limited to binding to the desired free site-specific cysteine site(s). Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions (preferably in combination with a stabilizing agent) are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In embodiments mild reducing agents may comprise compounds having one or more free thiols while in some embodiments mild reducing agents will comprise compounds having a single free thiol. Non-limiting examples of reducing agents compatible with the selective reduction techniques of the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a drug to an antibody may be determined by assessing the percentage of conjugation on the target conjugation site(s) (e.g. free cysteines on the c-terminus of each light chain) relative to all other conjugated sites. In certain embodiments, the method herein provides for efficiently conjugating a drug to an antibody comprising free cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that engineered antibodies capable of conjugation may contain free cysteine residues that comprise sulfhydryl groups that are blocked or capped as the antibody is produced or stored. Such caps include small molecules, proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed herein such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction).

D. DAR Distribution and Purification

In selected embodiments conjugation and purification methodology compatible with the present invention advantageously provides the ability to generate relatively homogeneous ADC preparations comprising a narrow DAR distribution. In this regard the disclosed constructs (e.g., site-specific constructs) and/or selective conjugation provides for homogeneity of the ADC species within a sample in terms of the stoichiometric ratio between the drug and the engineered antibody and with respect to the toxin location. As briefly discussed above the term “drug to antibody ratio” or “DAR” refers to the molar ratio of drug to antibody. In certain embodiments a conjugate preparation may be substantially homogeneous with respect to its DAR distribution, meaning that within the ADC preparation is a predominant species of site-specific ADC with a particular DAR (e.g., a DAR of 2 or 4) that is also uniform with respect to the site of loading (i.e., on the free cysteines). In other certain embodiments of the invention it is possible to achieve the desired homogeneity through the use of site-specific antibodies and/or selective reduction and conjugation. In other embodiments the desired homogeneity may be achieved through the use of site-specific constructs in combination with selective reduction. In yet other embodiments compatible preparations may be purified using analytical or preparative chromatography techniques to provide the desired homogeneity. In each of these embodiments the homogeneity of the ADC sample can be analyzed using various techniques known in the art including but not limited to mass spectrometry, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As discussed herein liquid chromatography methods such as reverse phase (RP) and hydrophobic interaction chromatography (HIC) may separate compounds in the mixture by drug loading value. In some cases, ion-exchange (IEC) or mixed-mode chromatography (MMC) may also be used to isolate species with a specific drug load.

The disclosed ADCs and preparations thereof may comprise drug and antibody moieties in various stoichiometric molar ratios depending on the configuration of the antibody and, at least in part, on the method used to effect conjugation. In certain embodiments the drug loading per ADC may comprise from 1-20 warheads (i.e., n is 1-20). Other selected embodiments may comprise ADCs with a drug loading of from 1 to 15 warheads. In still other embodiments the ADCs may comprise from 1-12 warheads or, more preferably, from 1-10 warheads. In some embodiments the ADCs will comprise from 1 to 8 warheads.

While theoretical drug loading may be relatively high, practical limitations such as free cysteine cross reactivity and warhead hydrophobicity tend to limit the generation of homogeneous preparations comprising such DAR due to aggregates and other contaminants. That is, higher drug loading, e.g. >8 or 10, may cause aggregation, insolubility, toxicity, or loss of cellular permeability of certain antibody-drug conjugates depending on the payload. In view of such concerns drug loading provided by the instant invention preferably ranges from 1 to 8 drugs per conjugate, i.e. where 1, 2, 3, 4, 5, 6, 7, or 8 drugs are covalently attached to each antibody (e.g., for IgG1, other antibodies may have different loading capacity depending the number of disulfide bonds). Preferably the DAR of compositions of the instant invention will be approximately 2, 4 or 6 and in some embodiments the DAR will comprise approximately 2.

Despite the relatively high level of homogeneity provided by the instant invention the disclosed compositions actually comprise a mixture of conjugates with a range of drugs compounds (potentially from 1 to 8 in the case of an IgG1). As such, the disclosed ADC compositions include mixtures of conjugates where most of the constituent antibodies are covalently linked to one or more drug moieties and (despite the relative conjugate specificity provided by engineered constructs and selective reduction) where the drug moieties may be attached to the antibody by various thiol groups. That is, following conjugation ADC compositions of the invention will comprise a mixture of conjugates with different drug loads (e.g., from 1 to 8 drugs per IgG1 antibody) at various concentrations (along with certain reaction contaminants primarily caused by free cysteine cross reactivity). However using selective reduction and post-fabrication purification the conjugate compositions may be driven to the point where they largely contain a single predominant desired ADC species (e.g., with a drug loading of 2) with relatively low levels of other ADC species (e.g., with a drug loading of 1, 4, 6, etc.). The average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting.

Thus, in some embodiments the present invention will comprise compositions having an average DAR of 1, 2, 3, 4, 5, 6, 7 or 8 each +/−0.5. In other embodiments the present invention will comprise an average DAR of 2, 4, 6 or 8+/−0.5. Finally, in selected embodiments the present invention will comprise an average DAR of 2+/−0.5 or 4+/−0.5. It will be appreciated that the range or deviation may be less than 0.4 in some embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other embodiments the ADC composition will comprise an average DAR of 2, 4, 6 or 8 each +/−0.4 with relatively low levels (<30%) of non-predominant ADC species. In some embodiments the ADC composition will comprise an average DAR of 2+/−0.4 with relatively low levels (<30%) of non-predominant ADC species. In yet other embodiments the predominant ADC species (e.g., DAR of 2 or DAR of 4) will be present at a concentration of greater than 50%, at a concentration of greater than 55%, at a concentration of greater than 60%, at a concentration of greater than 65%, at a concentration of greater than 70%, at a concentration of greater than 75%, at a concentration of greater that 80%, at a concentration of greater than 85%, at a concentration of greater than 90%, at a concentration of greater than 93%, at a concentration of greater than 95% or even at a concentration of greater than 97% when measured against all other DAR species present in the composition.

As detailed in the Examples below the distribution of drugs per antibody in preparations of ADC from conjugation reactions may be characterized by conventional means such as UV-Vis spectrophotometry, reverse phase HPLC, HIC, mass spectroscopy, ELISA, and electrophoresis. The quantitative distribution of ADC in terms of drugs per antibody may also be determined. By ELISA, the averaged value of the drugs per antibody in a particular preparation of ADC may be determined. However, the distribution of drug per antibody values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues.

### VI. PHARMACEUTICAL PREPARATIONS AND THERAPEUTIC USES

A. Formulations and Routes of Administration

The antibodies or ADCs of the invention can be formulated in various ways using art recognized techniques. In some embodiments, the therapeutic compositions of the invention can be administered neat or with a minimum of additional components while others may optionally be formulated to contain suitable pharmaceutically acceptable carriers. As used herein, “pharmaceutically acceptable carriers” comprise excipients, vehicles, adjuvants and diluents that are well known in the art and can be available from commercial sources for use in pharmaceutical preparation (see, e.g., Gennaro (2003) Remington: The Science and Practice of Pharmacy with Facts and Comparisons: Drugfacts Plus, 20th ed., Mack Publishing; Ansel et al. (2004) Pharmaceutical Dosage Forms and Drug Delivery Systems, 7^{th} ed., Lippencott Williams and Wilkins; Kibbe et al. (2000) Handbook of Pharmaceutical Excipients, 3^{rd} ed., Pharmaceutical Press.)

Suitable pharmaceutically acceptable carriers comprise substances that are relatively inert and can facilitate administration of the antibody or can aid processing of the active compounds into preparations that are pharmaceutically optimized for delivery to the site of action.

Such pharmaceutically acceptable carriers include agents that can alter the form, consistency, viscosity, pH, tonicity, stability, osmolarity, pharmacokinetics, protein aggregation or solubility of the formulation and include buffering agents, wetting agents, emulsifying agents, diluents, encapsulating agents and skin penetration enhancers. Certain non-limiting examples of carriers include saline, buffered saline, dextrose, arginine, sucrose, water, glycerol, ethanol, sorbitol, dextran, sodium carboxymethyl cellulose and combinations thereof. Antibodies for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington: The Science and Practice of Pharmacy (2000) 20th Ed. Mack Publishing.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additionally contain other pharmaceutically acceptable carriers, such as anti-oxidants, buffers, preservatives, stabilizers, bacteriostats, suspending agents, thickening agents, and solutes that render the formulation isotonic with the blood (or other relevant bodily fluid) of the intended recipient. Examples of excipients include, for example, water, alcohols, polyols, glycerol, vegetable oils, and the like. Examples of suitable isotonic pharmaceutically acceptable carriers for use in such formulations include Sodium Chloride Injection, Ringer's Solution, or Lactated Ringer's Injection.

Compatible formulations for parenteral administration (e.g., intravenous injection) may comprise ADC or antibody concentrations of from about 10 μg/mL to about 100 mg/mL. In certain selected embodiments antibody or ADC concentrations will comprise 20 μg/mL, 40 μg/mL, 60 μg/mL, 80 μg/mL, 100 μg/mL, 200 μg/mL, 300, μg/mL, 400 μg/mL, 500 μg/mL, 600 μg/mL, 700 μg/mL, 800 μg/mL, 900 μg/mL or 1 mg/mL. In other embodiments ADC concentrations will comprise 2 mg/mL, 3 mg/mL, 4 mg/mL, 5 mg/mL, 6 mg/mL, 8 mg/mL, 10 mg/mL, 12 mg/mL, 14 mg/mL, 16 mg/mL, 18 mg/mL, 20 mg/mL, 25 mg/mL, 30 mg/mL, 35 mg/mL, 40 mg/mL, 45 mg/mL, 50 mg/mL, 60 mg/mL, 70 mg/mL, 80 mg/mL, 90 mg/mL or 100 mg/mL.

The compounds and compositions of the invention may be administered in vivo, to a subject in need thereof, by various routes, including, but not limited to, oral, intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

B. Dosages and Dosing Regimens

The particular dosage regimen, i.e., dose, timing and repetition, will depend on the particular individual, as well as empirical considerations such as pharmacokinetics (e.g., half-life, clearance rate, etc.). Determination of the frequency of administration may be made by persons skilled in the art, such as an attending physician based on considerations of the condition and severity of the condition being treated, age and general state of health of the subject being treated and the like. Frequency of administration may be adjusted over the course of therapy based on assessment of the efficacy of the selected composition and the dosing regimen. Such assessment can be made on the basis of markers of the specific disease, disorder or condition. In embodiments where the individual has cancer, these include direct measurements of tumor size via palpation or visual observation; indirect measurement of tumor size by x-ray or other imaging techniques; an improvement as assessed by direct tumor biopsy and microscopic examination of a tumor sample; the measurement of an indirect tumor marker (e.g., PSA for prostate cancer) or an antigen identified according to the methods described herein; reduction in the number of proliferative or tumorigenic cells, maintenance of the reduction of such neoplastic cells; reduction of the proliferation of neoplastic cells; or delay in the development of metastasis.

The CLDN antibodies or ADCs of the invention may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the CLDN ADCs will be administered (preferably intravenously) at doses from about 0.001 mg/kg to about 1 g/kg. In certain embodiments the ADC may be administered at a concentration of 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.15 mg/kg, 0.16 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg or 1 g/kg. With the teachings herein one of skill in the art could readily determine appropriate dosages for various CLDN ADCs based on preclinical animal studies, clinical observations and standard medical and biochemical techniques and measurements.

Other dosing regimens may be predicated on Body Surface Area (BSA) calculations as disclosed in U.S. Ser. No. 14/509,809. As is well known, the BSA is calculated using the patient's height and weight and provides a measure of a subject's size as represented by the surface area of his or her body.

Anti-CLDN antibodies or ADCs may be administered on a specific schedule. Generally, an effective dose of the CLDN conjugate is administered to a subject one or more times. More particularly, an effective dose of the ADC is administered to the subject once a month, more than once a month, or less than once a month. In certain embodiments, the effective dose of the CLDN antibody or ADC may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the disclosed antibodies or ADCs.

In some embodiments the course of treatment involving conjugated antibodies will comprise multiple doses of the selected drug product over a period of weeks or months. More specifically, antibodies or ADCs of the instant invention may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices. The invention also contemplates discontinuous administration or daily doses divided into several partial administrations. The compositions of the instant invention and anti-cancer agent may be administered interchangeably, on alternate days or weeks; or a sequence of antibody treatments may be given, followed by one or more treatments of anti-cancer agent therapy. In any event, as will be understood by those of ordinary skill in the art, the appropriate doses of chemotherapeutic agents will be generally around those already employed in clinical therapies wherein the chemotherapeutics are administered alone or in combination with other chemotherapeutics.

In certain embodiments the present invention provides anti-CLDN antibody drug conjugates for use in the treatment of cancer wherein the treatment may comprise administering an effective amount of an anti-CLDN antibody drug conjugate (CLDN ADC) at least once every week (QW), at least once every two weeks (Q2W), at least once every three weeks (Q3W), at least once every four weeks (Q4W), at least once every five weeks (Q5W), at least once every six weeks (Q6W), at least once every seven weeks (Q7W), at least once every eight weeks (Q8W), at least once every nine weeks (Q9W) or at least once every ten weeks (Q10W). In selected embodiments the CLDN ADC will be administered at least every two weeks (Q2W), at least every three weeks (Q3W), at least once every four weeks (Q4W), at least once every five weeks (Q5W) or at least once every six weeks (Q6W). In other selected embodiments the CLDN ADC will be administered at a dose of about 0.05 mg/kg, 0.1 mg/kg, 0.2 mg/kg, 0.3 mg/kg, 0.4 mg/kg, 0.5 mg/kg, 0.6 mg/kg, 0.7 mg/kg or 0.8 mg/kg. Selected embodiments will comprise treating the patient with a single administration of the CLDN ADC. Certain other embodiments will comprise treating the patient at specified intervals (i.e. Q2W, Q3W, Q4W, Q5W, Q6W, etc) for two cycles (×2), for three cycles (×3), for four cycles (×4), for five cycles (×5) or for six cycles (×6). In other embodiments the initial CLDN ADC treatment (of x cycles) may be completed and no further CLDN ADC treatment is undertaken until the cancer shows signs of progressing (treatment at progression). In yet other embodiments the initial CLDN ADC treatment (of x cycles) may be completed and then the patient is put on maintenance therapy (e.g., 0.1 mg/kg CLDN ADC Q6W indefinitely).

In some aspects of the invention the CLDN ADC will comprise a PBD. In yet other aspects the CLDN ADC will be administered intravenously. In certain other aspects the cancer to be treated will comprise small cell lung cancer (SCLC) or large cell neuroendocrine cancer (LCNEC). In other selected aspects the cancer patients to be treated will comprise second line patients (i.e., previously treated patients). In yet other embodiments the cancer patients to be treated will comprise third line patients (i.e., patients that have been treated twice previously).

Certain preferred embodiments of the invention will comprise treating a patient with 0.2 mg/kg of CLDN ADC every 3 weeks for 3 cycles (0.2 mg/kg Q3W×3). In selected embodiments the patient to be treated at 0.2 mg/kg Q3W×3 will be suffering from SCLC. In other embodiments the patient to be treated at 0.2 mg/kg Q3W×3 will be suffering from LCNEC. In some aspects the patient has not been treated for the cancer. In certain aspects the patient will comprise a second line patient. In yet other embodiments the patient will comprise a third line patient. In other aspects the patient will be treated at progression following the 0.2 mg/kg Q3W×3 treatment cycle. In yet other aspects the patient will be shifted to CLDN ADC maintenance therapy following the 0.2 mg/kg Q3W×3 treatment cycle.

Certain other preferred embodiments of the invention will comprise treating a patient with 0.3 mg/kg of CLDN ADC every 6 weeks for 2 cycles (0.3 mg/kg Q6W×2). As shown below in the Examples such a regimen may be particularly effective (exhibit a efficacious therapeutic index) because of the relatively long half-life of the CLDN ADCs of the instant invention. In selected embodiments the patient to be treated at 0.3 mg/kg Q6W×2 will be suffering from SCLC. In other embodiments the patient to be treated at 0.3 mg/kg Q6W×2 will be suffering from LCNEC. In some aspects the patient has not been treated for the cancer. In certain aspects the patient will comprise a second line patient. In yet other embodiments the patient will comprise a third line patient. In other aspects the patient will be treated at progression following the 0.3 mg/kg Q6W×2 treatment cycle. In yet other aspects the patient will be shifted to CLDN ADC maintenance therapy following the 0.3 mg/kg Q6W×2 treatment cycle.

In further embodiments the CLDN ADCs of the instant invention may be administered at different dosages in any one cycle. For example, the drug may be administered (i.e, loaded or drug loading) at a relatively high dose (e.g., 0.5 mg/kg) followed by a lower dose of CLDN ADC (e.g., 0.2 mg/kg) four weeks later (Q4W) as part of the same cycle. Again such cycles may be repeated (2×, 3×, etc.) or delayed until progression (treat at progression) or followed up by CLDN ADC maintenance (e.g., 0.1 mg/kg Q4W indefinite).

In another embodiment the CLDN antibodies or ADCs of the instant invention may be used in maintenance therapy to reduce or eliminate the chance of tumor recurrence following the initial presentation of the disease. Such maintenance therapy may be used whether the first treatment was with CLDN ADC or another chemotherapeutic agent. Preferably the disorder will have been treated and the initial tumor mass eliminated, reduced or otherwise ameliorated so the patient is asymptomatic or in remission. At such time the subject may be administered pharmaceutically effective amounts of the disclosed ADCs one or more times even though there is little or no indication of disease using standard diagnostic procedures.

In another preferred embodiment the antibodies of the present invention may be used to prophylactically or as an adjuvant therapy to prevent or reduce the possibility of tumor metastasis following a debulking procedure. As used in the instant disclosure a “debulking procedure” means any procedure, technique or method that reduces, or ameliorates a tumor or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed ADCs may be administered as suggested by clinical, diagnostic or theragnostic procedures to reduce tumor metastasis.

Yet other embodiments of the invention comprise administering the disclosed ADCs to subjects that are asymptomatic but at risk of developing cancer. That is, the ADCs of the instant invention may be used in a truly preventative sense and given to patients that have been examined or tested and have one or more noted risk factors (e.g., genomic indications, family history, in vivo or in vitro test results, etc.) but have not developed neoplasia.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

C. Combination Therapies

The CLDN proteins are expressed in the tight junctions of epithelial cells where they are thought to establish the paracellular barrier that controls the flow of molecules in the intercellular space between epithelial cells. The use of anti-CLDN antibodies may result in the disruption of the tight junctions of epithelial cells and thus improve access of therapeutics that otherwise would not be able to penetrate cancer cells. Thus, the use of various therapies in combination with the anti-CLDN antibodies and ADCs of the invention may be useful in preventing or treating cancer and in preventing metastasis or recurrence of cancer. “Combination therapy”, as used herein, means the administration of a combination comprising at least one anti-CLDN antibody or ADC and at least one therapeutic moiety (e.g., anti-cancer agent) wherein the combination preferably has therapeutic synergy or improves the measurable therapeutic effects in the treatment of cancer over (i) the anti-CLDN antibody or ADC used alone, or (ii) the therapeutic moiety used alone, or (iii) the use of the therapeutic moiety in combination with another therapeutic moiety without the addition of an anti-CLDN antibody or ADC. The term “therapeutic synergy”, as used herein, means the combination of an anti-CLDN antibody or ADC and one or more therapeutic moiety(ies) having a therapeutic effect greater than the additive effect of the combination of the anti-CLDN antibody or ADC and the one or more therapeutic moiety(ies).

Desired outcomes of the disclosed combinations are quantified by comparison to a control or baseline measurement. As used herein, relative terms such as “improve,” “increase,” or “reduce” indicate values relative to a control, such as a measurement in the same individual prior to initiation of treatment described herein, or a measurement in a control individual (or multiple control individuals) in the absence of the anti-CLDN antibodies or ADCs described herein but in the presence of other therapeutic moiety(ies) such as standard of care treatment. A representative control individual is an individual afflicted with the same form of cancer as the individual being treated, who is about the same age as the individual being treated (to ensure that the stages of the disease in the treated individual and the control individual are comparable.)

Changes or improvements in response to therapy are generally statistically significant. As used herein, the term “significance” or “significant” relates to a statistical analysis of the probability that there is a non-random association between two or more entities. To determine whether or not a relationship is “significant” or has “significance,” a “p-value” can be calculated. P-values that fall below a user-defined cut-off point are regarded as significant. A p-value less than or equal to 0.1, less than 0.05, less than 0.01, less than 0.005, or less than 0.001 may be regarded as significant.

A synergistic therapeutic effect may be an effect of at least about two-fold greater than the therapeutic effect elicited by a single therapeutic moiety or anti-CLDN antibody or ADC, or the sum of the therapeutic effects elicited by the anti-CLDN antibody or ADC or the single therapeutic moiety(ies) of a given combination, or at least about five-fold greater, or at least about ten-fold greater, or at least about twenty-fold greater, or at least about fifty-fold greater, or at least about one hundred-fold greater. A synergistic therapeutic effect may also be observed as an increase in therapeutic effect of at least 10% compared to the therapeutic effect elicited by a single therapeutic moiety or anti-CLDN antibody or ADC, or the sum of the therapeutic effects elicited by the anti-CLDN antibody or ADC or the single therapeutic moiety(ies) of a given combination, or at least 20%, or at least 30%, or at least 40%, or at least 50%, or at least 60%, or at least 70%, or at least 80%, or at least 90%, or at least 100%, or more. A synergistic effect is also an effect that permits reduced dosing of therapeutic agents when they are used in combination.

In practicing combination therapy, the anti-CLDN antibody or ADC and therapeutic moiety(ies) may be administered to the subject simultaneously, either in a single composition, or as two or more distinct compositions using the same or different administration routes. Alternatively, treatment with the anti-CLDN antibody or ADC may precede or follow the therapeutic moiety treatment by, e.g., intervals ranging from minutes to weeks. In one embodiment, both the therapeutic moiety and the antibody or ADC are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the antibody and the therapeutic moiety.

The combination therapy can be administered until the condition is treated, palliated or cured on various schedules such as once, twice or three times daily, once every two days, once every three days, once weekly, once every two weeks, once every month, once every two months, once every three months, once every six months, or may be administered continuously. The antibody and therapeutic moiety(ies) may be administered on alternate days or weeks; or a sequence of anti-CLDN antibody or ADC treatments may be given, followed by one or more treatments with the additional therapeutic moiety. In one embodiment an anti-CLDN antibody or ADC is administered in combination with one or more therapeutic moiety(ies) for short treatment cycles. In other embodiments the combination treatment is administered for long treatment cycles. The combination therapy can be administered via any route.

In selected embodiments the compounds and compositions of the present invention may be used in conjunction with checkpoint inhibitors such as PD-1 inhibitors or PD-L1 inhibitors. PD-1, together with its ligand PD-L1, are negative regulators of the antitumor T lymphocyte response. In one embodiment the combination therapy may comprise the administration of anti-CLDN antibodies or ADCs together with an anti-PD-1 antibody (e.g. pembrolizumab, nivolumab, pidilizumab) and optionally one or more other therapeutic moiety(ies). In another embodiment the combination therapy may comprise the administration of anti-CLDN antibodies or ADCs together with an anti-PD-L1 antibody (e.g. avelumab, atezolizumab, durvalumab) and optionally one or more other therapeutic moiety(ies). In yet another embodiment, the combination therapy may comprise the administration of anti-CLDN antibodies or ADCs together with an anti PD-1 antibody or anti-PD-L1 administered to patients who continue progress following treatments with checkpoint inhibitors and/or targeted BRAF combination therapies (e.g. vemurafenib or dabrafinib).

1. Ovarian Cancer

Most patients with ovarian cancer have widespread disease at presentation. Although more than 80% of these women benefit from first-line therapy (which consists of aggressive tumor debulking and combination therapy with platinum-taxane regimen), tumor recurrence occurs in almost all these patients at a median of 15 months from diagnosis (Hennessy, Coleman, & Markman, 2009). Yearly mortality in ovarian cancer is approximately 65% of the incidence rate. Suboptimally debulked stage III and stage IV patients showed a 5-year survival rate lower than 10% with platinum-based combination therapy prior to the current generation of trials, including taxanes. Optimally debulked stage III and stage IV patients with a combination of intravenous taxane and intraperitoneal platinum plus taxane achieved a median survival of 66 months (Armstrong, et al., 2006).

Approximately 80% of patients diagnosed with ovarian epithelial, fallopian tube, and primary peritoneal cancer will relapse after first-line platinum-based and taxane-based chemotherapy. Clinical recurrences that take place within 6 months of completion of a platinum-containing regimen are considered platinum-refractory or platinum-resistant recurrences. Anthracyclines, taxanes, topotecan, etoposide and gemcitabine are used as single agents for these recurrences; however, response rates are modest (19-27%). In phase 2 studies, topotecan yielded overall response rates (“ORR”) as a single agent ranging from 13%-16.3%. Combination of weekly topotecan and biweekly bevacizumab showed an ORR rate of 25% in platinum-resistant patient population. Targeted therapies such bevacizumab and olaparib are available for patients not previously treated with bevacizumab and patients whose tumors test positive for deleterious BRCA1 or BRCA2 mutations, respectively. In phase 2 studies, single-agent bevacizumab yielded ORR ranging from 16%-21% in recurrent or platinum-resistant disease. Bevacizumab plus chemotherapy exhibited a median progression free survival (“PFS”) of 6.7 months compared to chemotherapy alone with an ORR rate of 30.9%. There was not statistically significant difference in OS between the regimens. In a phase 2 study, single-agent olaparib yielded a response rate of 34% and duration of response of 7.9 months in patients with platinum-resistant BRCA1 and 2 germline ovarian cancer. Olaparib is currently recommended for patients with advanced ovarian cancer who have received 3 or more lines of chemotherapy and who have germline BRCA mutation.

Thus in some embodiments, the anti-CLDN ADCs may be used in combination with various first line cancer treatments. In one embodiment the combination therapy comprises the use of an anti-CLDN antibody or ADC and a cytotoxic agent such as ifosfamide, mytomycin C, vindesine, vinblastine, etoposide, ironitecan, gemcitabine, taxanes, vinorelbine, methotrexate, and pemetrexed) and optionally one or more other therapeutic moiety(ies).

In another embodiment, for example in the treatment of ovarian cancer, the combination therapy comprises the use of an anti-CLDN antibody or ADC and bevacizumab and optionally one or more other therapeutic moiety(ies) (e.g. gemcitabine and/or a platinum analog).

In another embodiment the combination therapy comprises the use of an anti-CLDN antibody or ADC and a platinum-based drug (e.g. carboplatin or cisplatin) and optionally one or more other therapeutic moiety(ies) (e.g. vinorelbine; gemcitabine; a taxane such as, for example, docetaxel or paclitaxel; irinotican; or pemetrexed).

2. Breast Cancer

The ADCs of the invention can be used to treat breast cancer. In one aspect, the invention comprises a method of treating breast cancer (e.g. TNBC) comprising administering a pharmaceutical composition comprising an anti-CLDN ADC in combination with another therapeutic moiety disclosed herein. In one embodiment, for example, in the treatment of BR-ERPR, BR-ER or BR-PR cancer, the combination therapy comprises the use of an anti-CLDN antibody or ADC and one or more therapeutic moieties described as “hormone therapy”. “Hormone therapy” as used herein, refers to, e.g., tamoxifen; gonadotropin or luteinizing releasing hormone (GnRH or LHRH); everolimus and exemestane; toremifene; or aromatase inhibitors (e.g. anastrozole, letrozole, exemestane or fulvestrant).

In another embodiment, for example, in the treatment of BR-HER2, the combination therapy comprises the use of an anti-CLDN antibody or ADC and trastuzumab or ado-trastuzumab emtansine and optionally one or more other therapeutic moiety(ies) (e.g. pertuzumab and/or docetaxel).

In some embodiments, for example, in the treatment of metastatic breast cancer, the combination therapy comprises the use of an anti-CLDN antibody or ADC and a taxane (e.g. docetaxel or paclitaxel) and optionally an additional therapeutic moiety(ies), for example, an anthracycline (e.g. doxorubicin or epirubicin) and/or eribulin.

In another embodiment, for example, in the treatment of metastatic or recurrent breast cancer or BRCA-mutant breast cancer, the combination therapy comprises the use of an anti-CLDN antibody or ADC and megestrol and optionally an additional therapeutic moiety(ies).

In further embodiments, for example, in the treatment of BR-TNBC, the combination therapy comprises the use of an anti-CLDN antibody or ADC and a poly ADP ribose polymerase (PARP) inhibitor (e.g. BMN-673, olaparib, rucaparib and veliparib) and optionally an additional therapeutic moiety(ies).

In another embodiment, for example, in the treatment of breast cancer, the combination therapy comprises the use of an anti-CLDN antibody or ADC and cyclophosphamide and optionally an additional therapeutic moiety(ies) (e.g. doxorubicin, a taxane, epirubicin, 5-FU and/or methotrexate.

3. Lung Cancer

The ADCs of the invention can be used to treat breast cancer. In one aspect, the invention comprises a method of treating lung cancer (e.g. lung squamous cell carcinoma or lung adenocarcinoma) comprising administering a pharmaceutical composition comprising an anti-CLDN ADC in combination with another therapeutic moiety disclosed herein. In another embodiment combination therapy for the treatment of EGFR-positive NSCLC comprises the use of an anti-CLDN antibody or ADC and afatinib and optionally one or more other therapeutic moiety(ies) (e.g. erlotinib and/or bevacizumab).

In another embodiment combination therapy for the treatment of EGFR-positive NSCLC comprises the use of an anti-CLDN antibody or ADC and erlotinib and optionally one or more other therapeutic moiety(ies) (e.g. bevacizumab).

In another embodiment combination therapy for the treatment of ALK-positive NSCLC comprises the use of an anti-CLDN antibody or ADC and ceritinib and optionally one or more other therapeutic moiety(ies).

In another embodiment combination therapy for the treatment of ALK-positive NSCLC comprises the use of an anti-CLDN antibody or ADC and crizotinib and optionally one or more other therapeutic moiety(ies).

In another embodiment the combination therapy comprises the use of an anti-CLDN antibody or ADC and bevacizumab and optionally one or more other therapeutic moiety(ies) (e.g. a taxane such as, for example, docetaxel or paclitaxel; and/or a platinum analog).

In one embodiment the combination therapy comprises the use of an anti-CLDN antibody or ADC and platinum-based drug (e.g. carboplatin or cisplatin) analog and optionally one or more other therapeutic moiety(ies) (e.g. a taxane such as, for example, docetaxel and paclitaxel).

In one embodiment the combination therapy comprises the use of an anti-CLDN antibody or ADC and platinum-based drug (e.g. carboplatin or cisplatin) analog and optionally one or more other therapeutic moiety(ies) (e.g. a taxane such, for example, docetaxel and paclitaxel and/or gemcitabine and/or doxorubicin).

In a particular embodiment the combination therapy for the treatment of platinum-resistant tumors comprises the use of an anti-CLDN antibody or ADC and doxorubicin and/or etoposide and/or gemcitabine and/or vinorelbine and/or ifosfamide and/or leucovorin-modulated 5-fluoroucil and/or bevacizumab and/or tamoxifen; and optionally one or more other therapeutic moiety(ies).

In another embodiment the combination therapy comprises the use of an anti-CLDN antibody or ADC and a PARP inhibitor and optionally one or more other therapeutic moiety(ies).

In another embodiment the combination therapy comprises the use of an anti-CLDN antibody or ADC and bevacizumab and optionally cyclophosphamide.

The combination therapy may comprise an anti-CLDN antibody or ADC and a chemotherapeutic moiety that is effective on a tumor (e.g. melanoma) comprising a mutated or aberrantly expressed gene or protein (e.g. BRAF V600E).

T lymphocytes (e.g., cytotoxic lymphocytes (CTL)) play an important role in host defense against malignant tumors. CTL are activated by the presentation of tumor associated antigens on antigen presenting cells. Active specific immunotherapy is a method that can be used to augment the T lymphocyte response to cancer by vaccinating a patient with peptides derived from known cancer associated antigens. In one embodiment the combination therapy may comprise an anti-CLDN antibody or ADC and a vaccine to a cancer associated antigen (e.g. melanocyte-lineage specific antigen tyrosinase, gp100, Melan-A/MART-1 or gp75.) In other embodiments the combination therapy may comprise administration of an anti-CLDN antibody or ADC together with in vitro expansion, activation, and adoptive reintroduction of autologous CTLs or natural killer cells. CTL activation may also be promoted by strategies that enhance tumor antigen presentation by antigen presenting cells. Granulocyte macrophage colony stimulating factor (GM-CSF) promotes the recruitment of dendritic cells and activation of dendritic cell cross-priming. In one embodiment the combination therapy may comprise the isolation of antigen presenting cells, activation of such cells with stimulatory cytokines (e.g. GM-CSF), priming with tumor-associated antigens, and then adoptive reintroduction of the antigen presenting cells into patients in combination with the use of anti-CLDN antibodies or ADCs and optionally one or more different therapeutic moiety(ies).

The invention also provides for the combination of anti-CLDN antibodies or ADCs with radiotherapy. The term “radiotherapy”, as used herein, means, any mechanism for inducing DNA damage locally within tumor cells such as gamma-irradiation, X-rays, UV-irradiation, microwaves, electronic emissions and the like. Combination therapy using the directed delivery of radioisotopes to tumor cells is also contemplated, and may be used in combination or as a conjugate of the anti-CLDN antibodies disclosed herein. Typically, radiation therapy is administered in pulses over a period of time from about 1 to about 2 weeks. Optionally, the radiation therapy may be administered as a single dose or as multiple, sequential doses.

In other embodiments an anti-CLDN antibody or ADC may be used in combination with one or more of the anti-cancer agents described below.

D. Anti-Cancer Agents

The term “anti-cancer agent” or “chemotherapeutic agent” as used herein is one subset of “therapeutic moieties”, which in turn is a subset of the agents described as “pharmaceutically active moieties”. More particularly “anti-cancer agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, biological response modifiers, therapeutic antibodies, cancer vaccines, cytokines, hormone therapy, anti-metastatic agents and immunotherapeutic agents. It will be appreciated that in selected embodiments as discussed above, such anti-cancer agents may comprise conjugates and may be associated with antibodies prior to administration. In certain embodiments the disclosed anti-cancer agent will be linked to an antibody to provide an ADC as disclosed herein.

The term “cytotoxic agent”, which can also be an anti-cancer agent means a substance that is toxic to the cells and decreases or inhibits the function of cells and/or causes destruction of cells. Typically, the substance is a naturally occurring molecule derived from a living organism (or a synthetically prepared natural product). Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, mitegellin, restrictocin, phenomycin, neomycin, and the tricothecenes) or animals, (e.g., cytotoxic RNases, such as extracellular pancreatic RNases; DNase I, including fragments and/or variants thereof).

An anti-cancer agent can include any chemical agent that inhibits, or is designed to inhibit, a cancerous cell or a cell likely to become cancerous or generate tumorigenic progeny (e.g., tumorigenic cells). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. Such agents are often administered, and are often most effective, in combination, e.g., in the formulation CHOP. Again, in selected embodiments such anti-cancer agents may be conjugated to the disclosed antibodies to provide ADCs.

Examples of anti-cancer agents that may be used in combination with (or conjugated to) the antibodies of the invention include, but are not limited to, alkylating agents, alkyl sulfonates, anastrozole, amanitins, aziridines, ethylenimines and methylamelamines, acetogenins, a camptothecin, BEZ-235, bortezomib, bryostatin, callystatin, CC-1065, ceritinib, crizotinib, cryptophycins, dolastatin, duocarmycin, eleutherobin, erlotinib, pancratistatin, a sarcodictyin, spongistatin, nitrogen mustards, antibiotics, enediyne dynemicin, bisphosphonates, esperamicin, chromoprotein enediyne antibiotic chromophores, aclacinomysins, actinomycin, authramycin, azaserine, bleomycins, cactinomycin, canfosfamide, carabicin, carminomycin, carzinophilin, chromomycinis, cyclosphosphamide, dactinomycin, daunorubicin, detorubicin, 6-diazo-5-oxo-L-norleucine, doxorubicin, epirubicin, esorubicin, exemestane, fluorouracil, fulvestrant, gefitinib, idarubicin, lapatinib, letrozole, lonafarnib, marcellomycin, megestrol acetate, mitomycins, mycophenolic acid, nogalamycin, olivomycins, pazopanib, peplomycin, potfiromycin, puromycin, quelamycin, rapamycin, rodorubicin, sorafenib, streptonigrin, streptozocin, tamoxifen, tamoxifen citrate, temozolomide, tepodina, tipifarnib, tubercidin, ubenimex, vandetanib, vorozole, XL-147, zinostatin, zorubicin; anti-metabolites, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, polysaccharide complex, razoxane; rhizoxin; SF-1126, sizofiran; spirogermanium; tenuazonic acid; triaziquone; 2,2′,2″-trichlorotriethylamine; trichothecenes (T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, chloranbucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; XL518, inhibitors of PKC-alpha, Raf, H-Ras, EGFR and VEGF-A that reduce cell proliferation and pharmaceutically acceptable salts or solvates, acids or derivatives of any of the above. Also included in this definition are anti-hormonal agents that act to regulate or inhibit hormone action on tumors such as anti-estrogens and selective estrogen receptor antibodies, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts or solvates, acids or derivatives of any of the above.

Anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((S)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

The term “pharmaceutically acceptable salt” or “salt” means organic or inorganic salts of a molecule or macromolecule. Acid addition salts can be formed with amino groups. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′ methylene bis-(2-hydroxy 3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Where multiple charged atoms are part of the pharmaceutically acceptable salt, the salt can have multiple counter ions. Hence, a pharmaceutically acceptable salt can have one or more charged atoms and/or one or more counterion.

“Pharmaceutically acceptable solvate” or “solvate” refers to an association of one or more solvent molecules and a molecule or macromolecule. Examples of solvents that form pharmaceutically acceptable solvates include, but are not limited to, water, isopropanol, ethanol, methanol, DMSO, ethyl acetate, acetic acid, and ethanolamine.

In other embodiments the antibodies or ADCs of the instant invention may be used in combination with any one of a number of antibodies (or immunotherapeutic agents) presently in clinical trials or commercially available. The disclosed antibodies may be used in combination with an antibody selected from the group consisting of abagovomab, adecatumumab, afutuzumab, alemtuzumab, altumomab, amatuximab, anatumomab, arcitumomab, atezolizumab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nivolumab, nofetumomabn, obinutuzumab, ocaratuzumab, ofatumumab, olaratumab, olaparib, onartuzumab, oportuzumab, oregovomab, panitumumab, parsatuzumab, patritumab, pembrolizumab pemtumomab, pertuzumab, pidilizumab, pintumomab, pritumumab, racotumomab, radretumab, ramucirumab, rilotumumab, rituximab, robatumumab, satumomab, selumetinib, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8, MDX-1105 and MEDI4736 and combinations thereof.

Other embodiments comprise the use of antibodies approved for cancer therapy including, but not limited to, rituximab, gemtuzumab ozogamcin, alemtuzumab, ibritumomab tiuxetan, tositumomab, bevacizumab, cetuximab, patitumumab, ofatumumab, ipilimumab and brentuximab vedotin. Those skilled in the art will be able to readily identify additional anti-cancer agents that are compatible with the teachings herein.

E. Radiotherapy

The present invention also provides for the combination of antibodies or ADCs with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed antibodies or ADCs may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### VII. INDICATIONS

The invention provides for the use of antibodies and ADCs of the invention for the diagnosis, theragnosis, treatment and/or prophylaxis of various disorders including neoplastic, inflammatory, angiogenic and immunologic disorders and disorders caused by pathogens. In certain embodiments the diseases to be treated comprise neoplastic conditions comprising solid tumors. In other embodiments the diseases to be treated comprise hematologic malignancies. In certain embodiments the antibodies or ADCs of the invention will be used to treat tumors or tumorigenic cells expressing a CLDN determinant. Preferably the “subject” or “patient” to be treated will be human although, as used herein, the terms are expressly held to comprise any mammalian species.

It will be appreciated that the compounds and compositions of the instant invention may be used to treat subjects at various stages of disease and at different points in their treatment cycle. Accordingly, in certain embodiments the antibodies and ADCs of the instant invention will be used as a front line therapy and administered to subjects who have not previously been treated for the cancerous condition. In other embodiments the antibodies and ADCs of the invention will be used to treat second and third line patients (i.e., those subjects that have previously been treated for the same condition one or two times respectively). Still other embodiments will comprise the treatment of fourth line or higher patients (e.g., gastric or colorectal cancer patients) that have been treated for the same or related condition three or more times with the disclosed CLDN ADCs or with different therapeutic agents. In other embodiments the compounds and compositions of the present invention will be used to treat subjects that have previously been treated (with antibodies or ADCs of the present invention or with other anti-cancer agents) and have relapsed or are determined to be refractory to the previous treatment. In selected embodiments the compounds and compositions of the instant invention may be used to treat subjects that have recurrent tumors.

In certain embodiments the compounds and compositions of the instant invention will be used as a front line or induction therapy either as a single agent or in combination and administered to subjects who have not previously been treated for the cancerous condition. In other embodiments the compounds and compositions of the present invention will be used during consolidation or maintenance therapy as either a single agent or in combination. In other embodiments the compounds and compositions of the present invention will be used to treat subjects that have previously been treated (with antibodies or ADCs of the present invention or with other anti-cancer agents) and have relapsed or determined to be refractory to the previous treatment. In selected embodiments the compounds and compositions of the instant invention may be used to treat subjects that have recurrent tumors. In other embodiments the compounds and compositions of the present invention will be used as part of a conditioning regimen in preparation of receiving either an autologous or allogeneic hematopoietic stem cell transplant with bone marrow, cord blood or mobilized peripheral blood as the stem cell source.

More generally neoplastic conditions subject to treatment in accordance with the instant invention may be benign or malignant; solid tumors or hematologic malignancies; and may be selected from the group including, but not limited to: adrenal gland tumors, AIDS-associated cancers, alveolar soft part sarcoma, astrocytic tumors, autonomic ganglia tumors, bladder cancer (squamous cell carcinoma and transitional cell carcinoma), blastocoelic disorders, bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, epithelial disorders, Ewing's tumors, extraskeletal myxoid chondrosarcoma, fibrogenesis imperfecta ossium, fibrous dysplasia of the bone, gallbladder and bile duct cancers, gastric cancer, gastrointestinal, gestational trophoblastic disease, germ cell tumors, glandular disorders, head and neck cancers, hypothalamic, intestinal cancer, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), macrophagal disorders, medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, stromal disorders, synovial sarcoma, testicular cancer, thymic carcinoma, thymoma, thyroid metastatic cancer, and uterine cancers (carcinoma of the cervix, endometrial carcinoma, and leiomyoma).

In certain embodiments the compounds and compositions of the instant invention will be used as a front line therapy and administered to subjects who have not previously been treated for the cancerous condition. In other embodiments the compounds and compositions of the present invention will be used to treat subjects that have previously been treated (with antibodies or ADCs of the present invention or with other anti-cancer agents) and have relapsed or determined to be refractory to the previous treatment. In selected embodiments the compounds and compositions of the instant invention may be used to treat subjects that have recurrent tumors.

In certain embodiments the proliferative disorder will comprise a solid tumor including, but not limited to, adrenal, liver, kidney, bladder, breast, gastric, ovarian, endometrial, cervical, uterine, esophageal, colorectal, prostate, pancreatic, lung (both small cell and non-small cell), thyroid, carcinomas, sarcomas, glioblastomas and various head and neck tumors. In other embodiments, and as shown in the Examples below, the disclosed ADCs are especially effective at treating small cell lung cancer (SCLC) and non-small cell lung cancer (NSCLC) (e.g., squamous cell non-small cell lung cancer or squamous cell small cell lung cancer). In one embodiment, the lung cancer is refractory, relapsed or resistant to a platinum based agent (e.g., carboplatin, cisplatin, oxaliplatin, topotecan) and/or a taxane (e.g., docetaxel, paclitaxel, larotaxel or cabazitaxel). In another embodiment the subject to be treated is suffering from large cell neuroendocrine carcinoma (LCNEC). In selected embodiments the antibodies and ADCs can be administered to patients exhibiting limited stage disease or extensive stage disease. In other embodiments the disclosed conjugated antibodies will be administered to refractory patients (i.e., those whose disease recurs during or shortly after completing a course of initial therapy); sensitive patients (i.e., those whose relapse is longer than 2-3 months after primary therapy); or patients exhibiting resistance to a platinum based agent (e.g. carboplatin, cisplatin, oxaliplatin) and/or a taxane (e.g. docetaxel, paclitaxel, larotaxel or cabazitaxel). In certain preferred embodiments the CLDN ADCs of the instant invention may be administered to frontline patients. In other embodiments the CLDN ADCs of the instant invention may be administered to second line patients. In still other embodiments the CLDN ADCs of the instant invention may be administered to third line patients.

A. Gynecological Cancers

In certain embodiments the ADCs of the invention are used to treat gynecologic cancers, particularly ovarian cancer or uterine endometrial cancers. Ovarian cancer represents 1.3% of all new cancer cases diagnosed in the United States with an estimated 21,290 new cases and 14,180 deaths in 2015. Epithelial carcinoma of the ovary is one of the most common gynecologic malignancies and the fifth most frequent cause of cancer death in women, with 50% of all cases occurring in women older than 65 years. Less than 40% of patients with epithelial ovarian cancer are cured. Although less common, fallopian tube cancer and primary peritoneal cancer are similar to ovarian epithelial cancer and are staged and treated in the same way.

The main subtypes of ovarian carcinoma include high- and low-grade serous, endometroid, clear-cell, and mucinous. Clear-cell, low-grade endometroid, mucinous, and low-grade serous carcinomas originate from atypical endometriosis or from borderline serous tumors, are characterized by specific mutations in K-Ras, B-Raf, ERBB2, CTNNB1, PTEN, ARID1A and HNF1 and have intermediate to favorable prognoses. High-grade serous carcinomas account for approximately 70% of all ovarian cancer diagnoses with most patients having advanced disease (Stage III and IV) at the time of diagnosis and poor prognosis. These tumors are thought to originate from the fimbriated epithelium at the end of the fallopian tube, are genetically unstable, and almost all are associated with TP53 mutation and/or dysfunction resulting in either accumulation or complete loss of p53 protein. BRCA1 and 2 germline and somatic mutations are associated with high-grade serous tumors and occur in ˜15% and 6% of cases of ovarian cancer, respectively.

Uterine corpus endometrial carcinoma is the most common gynecological malignancy in the United States, accounting for about 6% of all cancers in women, with an estimated 60,050 new cases and 10, 470 deaths in 2016. This type of gynecological malignancy begins in the endometrium, the inner lining of the uterus. It occurs most commonly in women aged 60 and over. Almost 70% of endometrial cancers are diagnosed at early stage, where the cancer does not extend beyond the uterus. Later stage tumors that have spread beyond the uterus may be treated with hormone therapy, provided these tumors express the appropriate receptors. A subset of uterine corpus endometrial carcinomas share genetic features with serous ovarian cancers, including frequent mutations in TP53, few DNA methylation changes, and extensive copy number alterations.

Thus in further embodiments the invention comprises a method of treating ovarian cancer, e.g. high- and low-grade serous, endometroid, clear-cell, and mucinous ovarian carcinoma, comprising administering a pharmaceutical composition comprising an anti-CLDN ADC disclosed herein. In other embodiments, the invention comprises a method of treating uterine endometrial cancer, particularly later stage (e.g., stage III and stage IV), endometrial cancers.

B. Lung Cancer

In other embodiments, the disclosed antibodies and ADCs are especially effective at treating lung cancer, including the following subtypes: small cell lung cancer and non-small cell lung cancer (e.g. squamous cell, adenocarcinoma).

In some embodiments the disclosed ADCs may be used to treat small cell lung cancer. With regard to such embodiments the conjugated antibodies may be administered to patients exhibiting limited stage disease. In other embodiments the disclosed ADCs will be administered to patients exhibiting extensive stage disease. In other preferred embodiments the disclosed ADCs will be administered to refractory patients (i.e., those who recur during or shortly after completing a course of initial therapy) or recurrent small cell lung cancer patients. Still other embodiments comprise the administration of the disclosed ADCs to sensitive patients (i.e., those whose relapse is longer than 2-3 months after primary therapy. In each case it will be appreciated that compatible ADCs may be used in combination with other anti-cancer agents depending on the selected dosing regimen and the clinical diagnosis. The anti-CLDN ADCs of the invention may also be used to treat SCLC patients with progressive disease after one or two treatments (i.e., second or third line SCLC patients). In some embodiments the disclosed ADCs may be used to treat small cell lung cancer. With regard to such embodiments the conjugated antibodies may be administered to patients exhibiting limited stage disease. In other embodiments the disclosed ADCs will be administered to patients exhibiting extensive stage disease. In other preferred embodiments the disclosed ADCs will be administered to refractory patients (i.e., those who recur during or shortly after completing a course of initial therapy) or recurrent small cell lung cancer patients. Still other embodiments comprise the administration of the disclosed ADCs to sensitive patients (i.e., those whose relapse is longer than 2-3 months after primary therapy. In each case it will be appreciated that compatible ADCs may be used in combination with other anti-cancer agents depending the selected dosing regimen and the clinical diagnosis. The anti-CLDN ADCs of the invention may also be used to treat SCLC patients with progressive disease after one or two treatments (i.e., second or third line SCLC patients).

C. Breast Cancer

In other embodiments, the disclosed antibodies and ADCs are especially effective at treating breast cancer, e.g., basal-like, endometrial, estrogen receptor positive and/or progesterone receptor positive, triple negative breast cancer. The ADCs may be administered to patients exhibiting limited stage disease or extensive stage disease. In other embodiments the disclosed ADCs will be administered to refractory patients or recurrent breast cancer patients. Still other embodiments comprise the administration of the disclosed ADCs to sensitive patients suffering from breast cancer. In each case it will be appreciated that compatible anti-CLDN ADCs may be used in combination with other anti-cancer agents depending the selected dosing regimen and the clinical diagnosis.

### VIII. ARTICLES OF MANUFACTURE

The invention includes pharmaceutical packs and kits comprising one or more containers or receptacles, wherein a container can comprise one or more doses of an antibody or ADC of the invention. Such kits or packs may be diagnostic or therapeutic in nature. In certain embodiments, the pack or kit contains a unit dosage, meaning a predetermined amount of a composition comprising, for example, an antibody or ADC of the invention, with or without one or more additional agents and optionally, one or more anti-cancer agents. In certain other embodiments, the pack or kit contains a detectable amount of an anti-CLDN antibody or ADC, with or without an associated reporter molecule and optionally one or more additional agents for the detection, quantitation and/or visualization of cancerous cells.

In any event kits of the invention will generally comprise an antibody or ADC of the invention in a suitable container or receptacle a pharmaceutically acceptable formulation and, optionally, one or more anti-cancer agents in the same or different containers. The kits may also contain other pharmaceutically acceptable formulations or devices, either for diagnosis or combination therapy. Examples of diagnostic devices or instruments include those that can be used to detect, monitor, quantify or profile cells or markers associated with proliferative disorders (for a full list of such markers, see above). In some embodiments the devices may be used to detect, monitor and/or quantify circulating tumor cells either in vivo or in vitro (see, for example, WO 2012/0128801). In still other embodiments the circulating tumor cells may comprise tumorigenic cells. The kits contemplated by the invention can also contain appropriate reagents to combine the antibody or ADC of the invention with an anti-cancer agent or diagnostic agent (e.g., see U.S. Pat. No. 7,422,739).

When the components of the kit are provided in one or more liquid solutions, the liquid solution can be non-aqueous, though typically an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the antibody or ADC of the invention in combination with additional therapeutics or agents, the solution may be pre-mixed, either in a molar equivalent combination, or with one component in excess of the other. Alternatively, the antibody or ADC of the invention and any optional anti-cancer agent or other agent (e.g., steroids) can be maintained separately within distinct containers prior to administration to a patient.

In certain preferred embodiments the aforementioned kits comprising compositions of the invention will comprise a label, marker, package insert, bar code and/or reader indicating that the kit contents may be used for the treatment, prevention and/or diagnosis of cancer. In other preferred embodiments the kit may comprise a label, marker, package insert, bar code and/or reader indicating that the kit contents may be administered in accordance with a certain dosage or dosing regimen to treat a subject suffering from cancer. In a particularly preferred aspect the label, marker, package insert, bar code and/or reader indicates that the kit contents may be used for the treatment, prevention and/or diagnosis of a hematologic malignancy (e.g., AML) or provide dosages or a dosing regimen for treatment of the same. In other particularly preferred aspects the label, marker, package insert, bar code and/or reader indicates that the kit contents may be used for the treatment, prevention and/or diagnosis of lung cancer (e.g., adenocarcinoma) or a dosing regimen for treatment of the same.

Suitable containers or receptacles include, for example, bottles, vials, syringes, infusion bags (i.v. bags), etc. The containers can be formed from a variety of materials such as glass or pharmaceutically compatible plastics. In certain embodiments the receptacle(s) can comprise a sterile access port. For example, the container may be an intravenous solution bag or a vial having a stopper that can be pierced by a hypodermic injection needle.

In some embodiments the kit can contain a means by which to administer the antibody and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

### IX. MISCELLANEOUS

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well-known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### X. REFERENCES

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PBD, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described.

### EXAMPLES

The invention, generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Listing Summaries

TABLE 3 provides a summary of amino acid and nucleic acid sequences included herein.

TABLE 3

| SEQ ID NO | Description |
|---|---|
| 1 | IgG1 heavy chain constant region protein |
| 2 | C220S IgG1 heavy constant region protein |
| 3 | C220Δ IgG1 heavy constant region protein |
| 4 | kappa light chain constant region protein |
| 5 | C214S kappa light chain constant region protein |
| 6 | C214Δ kappa light chain constant region protein |
| 7 | lambda light chain constant region protein |
| 8 | C214S lambda light chain constant region protein |
| 9 | C214Δ lambda light chain constant region protein |
| 10 | Protein sequence of CLDN6 |
| 11 | Protein sequence of CLDN9 |
| 12-19 | Reserved |
| 20 | SC27.1 VL DNA |
| 21 | SC27.1 VL protein |
| 22 | SC27.1 VH DNA |
| 23 | SC27.1 VH protein |
| 24-59 | Additional mouse clones as in SEQ ID NOs: 20-23 |
| 60-61 | hSC27.1 VL DNA and VL protein |
| 62-63 | hSC27.1 VH DNA and VH protein |
| 64-65 | hSC27.22 VL DNA and VL protein |
| 66-67 | hSC27.22 VH DNA and VH protein |
| 68-69 | hSC27.108 VL DNA and VL protein |
| 70-71 | hSC27.108 VH DNA and VH protein |
| 72-73 | hSC27.204 VL DNA and VL protein |
| 74-75 | hSC27.204 VH DNA and VH protein |
| 76-77 | hSC27.204v2 VH DNA and VH protein |
| 78-79 | hSC27.1 full length light and heavy chain protein |
| | sequence |
| 80-81 | hSC27.22 full length light and heavy chain protein |
| | sequence |
| 82 | hSC27.22ss1 full length heavy chain protein sequence |
| 83-84 | hSC27.108 full length light and heavy chain protein |
| | sequence |
| 85 | hSC27.108ss1 full length heavy chain protein sequence |
| 86-87 | hSC27.204 full length light and heavy chain protein |
| | sequence |
| 88 | hSC27.204v2 full length heavy chain protein sequence |
| 89 | hSC27.204v2ss1 full length heavy chain protein sequence |
| 90-95 | CDRL1-CDRL3 and CDRH1-CDRH3 of hSC27.1 |
| 96-101 | CDRL1-CDRL3 and CDRH1-CDRH3 of hSC27.22 |
| 102-107 | CDRL1-CDRL3 and CDRH1-CDRH3 of hSC27.108 |
| 108 | Reserved |
| 109-114 | CDRL1-CDRL3 and CDRH1-CDRH3 of hSC27.204 |
| 115 | CDRH2 of hSC27.204v2 |

### Tumor Cell Line Summary

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in TABLE 4 as follows:

TABLE 4

| | Abbre- | | |
|---|---|---|---|
| Tumor Type | viation | Tumor subtype | Abbreviation |
| Bladder | BL | | |
| Breast | BR | | |
| | | basal-like | BR-Basal Like |
| | | endometrial | BR-END |
| | | estrogen receptor positive | BR-ERPR |
| | | and/or progesterone | |
| | | receptor positive | |
| | | ERBB2/Neu positive | BR- ERBB2/Neu |
| | | HER2 positive | BR-HER2 |
| | | triple-negative | TNBC |
| | | luminal A | BR-lumA |
| | | claudin subtype of | TNBC-CL |
| | | triple-negative | |
| | | claudin low | BR-CLDN-Low |
| Cervical | CER | | |
| Colorectal | CR | | |
| Endometrial | EM | | |
| | EM-Ad | endometrial | |
| | | adenocarcinoma | |
| Gastric | GA | | |
| | | diffuse adenocarcinoma | GA-Ad-Dif/Muc |
| | | intestinal adenocarcinoma | GA-Ad-Int |
| | | stromal tumors | GA-GIST |
| Glioblastoma | GB | | |
| Head and neck | HN | | |
| Kidney | KDY | | |
| | | clear renal cell carcinoma | KDY-CC |
| | | papillary renal cell | KDY-PAP |
| | | carcinoma | |
| | | transitional cell or | KDY-URO |
| | | urothelial carcinoma | |
| | | unknown | KDY-UNK |
| Liver | LIV | | |
| | | hepatocellular carcinoma | LIV-HCC |
| | | cholangiocarcinoma | LIV-CHOL |
| Lung | LU | | |
| | | adenocarcinoma | LU-Ad |
| | | carcinoid | LU-CAR |
| | | large cell neuroendocrine | LU-LCC |
| | | non-small cell | NSCLC |
| | | squamous cell | LU-SCC |
| | | small cell | SCLC |
| | | spindle cell | LU-SPC |
| Lymphoma | LN | | |
| Ovarian | OV | | |
| | | clear cell | OV-CC |
| | | endometrioid | OV-END |
| | | endometrioid | OV-END-Ad |
| | | adenocarcinoma | |
| | | mixed subtype | OV-MIX |
| | | malignant mixed | OV-MMMT |
| | | mesodermal | |
| | | mucinous | OV-MUC |
| | | neuroendocrine | OV-NET |
| | | papillary serous | OV-PS |
| | | serous | OV-S |
| | | small cell | OV-SC |
| | | transitional cell | OV-TCC |
| | | carcinoma | |
| Pancreatic | PA | | |
| | | acinar cell carcinoma | PA-ACC |
| | | duodenal carcinoma | PA-DC |
| | | mucinous adenocarcinoma | PA-MAD |
| | | neuroendocrine | PA-NET |
| | | adenocarcinoma | PA-PAC |
| | | adenocarcinoma exocrine | PA-PACe |
| | | type | |
| | | ductal adenocarcinoma | PA-PDAC |
| | | ampullary adenocarcinoma | PA-AAC |
| Prostate | PR | | |
| Skin | SK | | |
| | | melanoma | MEL |
| | | squamous cell carcinomas | SK-SCC |
| | | uveal melanoma | UVM |
| Testicular | TES | | |
| Thyroid | THY | | |
| Uterine | UT | | |
| | | Uterine corpus endometrial | UTEC |
| | | carcinoma | |

### Example 1

### Cloning and Expression of Recombinant CLDN Proteins and Engineering of Cell Lines Overexpressing Cell Surface CLDN Proteins

The human claudin (CLDN) gene family is comprised of 23 known genes. In order to deduce the relationships between claudin protein sequences, the AlignX program of the Vector NTI software package was used to align 30 claudin protein sequences from 23 human CLDN genes. The results of this alignment are depicted as a dendrogram in FIG. 1A. A review of the figure shows that CLDN6 and CLDN9 are very closely related in sequence, appearing adjacent to one another on the same branch of the dendrogram, while CLDN4 is the next most closely related CLDN protein sequence. Examination of the amino acid sequences themselves shows that the human CLDN6 protein is very closely related to the human CLDN9 protein sequence (FIG. 1B). Closer inspection reveals that CLDN6 and CLDN9 proteins are highly conserved in their extracellular domain (ECDs), (bold, FIG. 1B), while the carboxy-terminal cytoplasmic domain is the most divergent portion of these proteins (lower case, residues 181-220, FIG. 1B). Based upon these protein sequence relationships, it was hypothesized that immunization with a full length human CLDN6 antigen would yield many antibodies recognizing the human CLDN6 ECD that will also be cross-reactive with the human CLDN9 ECD.

DNA Fragments Encoding Human CLDN6, CLDN4, and CLDN9 Proteins.

To generate all molecular and cellular materials required in the present invention pertaining to the human CLDN6 (hCLDN6) protein, a codon-optimized DNA fragment encoding a protein identical to NCBI protein accession NP_067018 was synthesized (IDT). This DNA clone was used for all subsequent engineering of constructs expressing the mature hCLDN6 protein or fragments thereof. Similarly, codon-optimized DNA fragments encoding proteins identical to NCBI protein accession NP_001296 for human CLDN4 (hCLDN4), or NCBI protein accession NP_066192 for human CLDN9 (hCLDN9) were purchased and used for all subsequent engineering of constructs expressing the hCLDN4 or hCLDN9 proteins or fragments thereof.

Cell Line Engineering

Engineered cell lines overexpressing the various CLDN proteins listed above were constructed using lentiviral vectors to transduce HEK293T or 3T3 cell lines using art recognized techniques. First, PCR was used to amplify the DNA fragments encoding the protein of interest (e.g., hCLDN6, hCLDN9, or hCLDN4) using the commercially synthesized DNA fragments described above as templates. Then, the individual PCR products were subcloned into the multiple cloning site (MCS) of the lentiviral expression vector, pCDH-EF1-MCS-T2A-GFP (System Biosciences), to generate a suite of lentiviral vectors. The T2A sequence in resultant pCDH-EF1-CLDN-T2A-GFP vectors promotes ribosomal skipping of a peptide bond condensation, resulting in expression of two independent proteins: high level expression of the specific CLDN protein encoded upstream of the T2A peptide, with co-expression of the GFP marker protein encoded downstream of the T2A peptide. This suite of lentiviral vectors was used to create separate stable HEK293T or 3T3 cell lines overexpressing individual CLDN proteins using standard lentiviral transduction techniques well known to those skilled in the art. CLDN-positive cells were selected with FACS using high-expressing HEK293T subclones, which were also strongly positive for GFP.

### Example 2

### Generation of Anti-CLDN Antibodies

Two immunizations were performed for the purpose of generating antibodies that recognize CLDN proteins. In the first immunization, mice were inoculated with HEK293T cells or 3T3 cells overexpressing hCLDN6 (generated as described in Example 1). In the first immunization, six mice (two each of the following strains: Balb/c, CD-1, FVB) were inoculated with 1 million hCLDN6-HEK293T cells emulsified with an equal volume of adjuvant. In the second immunization six mice (two each of the following strains: Balb/c, CD-1, FVB) were inoculated with 3T3 cells overexpressing CLDN6. Following the initial inoculation in each case, the mice were injected twice weekly for seven weeks with the respective inoculums.

Mice were sacrificed and draining lymph nodes (popliteal, inguinal, and medial iliac) were dissected and used as a source for antibody producing cells. A single cell suspension of B cells (305×10⁶ cells) were fused with non-secreting P3x63Ag8.653 myeloma cells (ATCC #CRL-1580) at a ratio of 1:1 by electro cell fusion using a model BTX Hybrimmune System (BTX Harvard Apparatus). Cells were resuspended in hybridoma selection medium: DMEM medium (Cellgro) supplemented with azaserine (Sigma), 15% fetal clone I serum (Hyclone), 10% BM condimed (Roche Applied Sciences), 1 mM sodium pyruvate, 4 mM L-glutamine, 100 IU penicillin-streptomycin, 50 μM 2-mercaptoethanol, and 100 μM hypoxanthine, and cultured in three T225 flasks in 90 mL selection medium per flask. The flasks were placed in a humidified 37° C. incubator containing 5% CO₂ and 95% air for 6 days. The library was frozen down in 6 vials of CryoStor CS10 buffer (BioLife Solutions), with approximately 15×10⁶ viable cells per vial, and stored in liquid nitrogen.

One vial from the library was thawed at 37° C. and the frozen hybridoma cells were added to 90 mL hybridoma selection medium, described above, and placed in a T150 flask. The cells were cultured overnight in a humidified 37° C. incubator with 5% CO₂ and 95% air. The following day hybridoma cells were collected from the flask and plated at one cell per well (using a FACSAria I cell sorter) in 200 μL of supplemented hybridoma selection medium into 48 Falcon 96-well U-bottom plates. The hybridomas were cultured for 10 days and the supernatants were screened for antibodies specific to hCLDN6, hCLDN4 or hCLDN9 proteins using flow cytometry. Flow cytometry was performed as follows: 1×10⁵ per well of HEK293T cells, stably transduced with lentiviral vectors encoding hCLDN6, hCLDN4 or hCLDN9, were incubated for 30 mins. with 100 μL hybridoma supernatent. Cells were washed with PBS/2% FCS and then incubated with 50 μL per sample DyeLight 649 labeled goat-anti-mouse IgG, Fc fragment specific secondary antibody diluted 1:200 in PBS/2% FCS. After a 15 min. incubation cells were washed twice with PBS/2% FCS and re-suspended in PBS/2% FCS with DAPI (to detect dead cells) and analyzed by flow cytometry for fluorescence exceeding that of cells stained with an isotype control antibody. Selected hybridomas that tested positive for antibodies directed to one or more of the CLDN antigens were set aside for further characterization. Remaining, unused hybridoma library cells were frozen in liquid nitrogen for future library testing and screening.

### Example 3

### Sequencing of Anti-CLDN Antibodies

Anti-CLDN antibodies were generated as described in Example 2 above and then sequenced as follows. Total RNA was purified from selected hybridoma cells using the RNeasy Miniprep Kit (Qiagen) according to the manufacturer's instructions. Between 10⁴ and 10⁵ cells were used per sample. Isolated RNA samples were stored at −80° C. until used. The variable region of the Ig heavy chain of each hybridoma was amplified using two 5′ primer mixes comprising 86 mouse specific leader sequence primers designed to target the complete mouse VH repertoire in combination with a 3′ mouse Cγ primer specific for all mouse Ig isotypes. Similarly, two primer mixes containing 64 5′ VK leader sequences designed to amplify each of the VK mouse families was used in combination with a single reverse primer specific to the mouse kappa constant region in order to amplify and sequence the kappa light chain. The VH and VL transcripts were amplified from 100 ng total RNA using the Qiagen One Step RT-PCR kit as follows. A total of four RT-PCR reactions were run for each hybridoma, two for the VK light chain and two for the VH heavy chain. PCR reaction mixtures included 1.5 μL of RNA, 0.4 μL of 100 μM of either heavy chain or kappa light chain primers (custom synthesized by IDT), 5 μL of 5×RT-PCR buffer, 1 μL dNTPs, and 0.6 μL of enzyme mix containing reverse transcriptase and DNA polymerase. The thermal cycler program included the following steps: RT step 50° C. for 60 min., 95° C. for 15 min. followed by 35 cycles of (94.5° C. for 30 seconds, 57° C. for 30 seconds, 72° C. for 1 min.), and a final incubation at 72° C. for 10 min. The extracted PCR products were sequenced using the same specific variable region primers as described above. PCR products were sent to an external sequencing vendor (MCLAB) for PCR purification and sequencing services.

FIGS. 2A and 2B show light chain (FIG. 2A) and heavy chain (FIG. 2B) variable region amino acid sequences of exemplary mouse and humanized (described in Example 4 below) anti-CLDN antibodies (SEQ ID NOS: 21-77, odd numbers) and variants of hSC27.22, hSC27.108 and hSC27.204 (as further described in Example 5 below). Mouse and humanized light and heavy chain variable region nucleic acid sequences are provided in FIG. 2C (SEQ ID NOS: 20-76, even numbers). Taken together FIGS. 2A and 2B provide annotated VH and VL sequences of mouse and humanized anti-CLDN antibodies, termed SC27.1, SC27.22, SC27.103, SC27.104, SC27.105, SC27.106, SC27.108, SC27.201, SC27.203 SC27.204, hSC27.1, hSC27.22, hSC27.108, hSC27.204 and hSC27.204v2. The amino acid sequences are annotated to identify the framework regions (i.e. FR1-FR4) and the complementarity determining regions (i.e. CDRL1-CDRL3 in FIG. 2A or CDRH1-CDRH3 in FIG. 2B) defined as per Kabat. FIGS. 2E-2H show annotated amino acid sequences (numbered as per Kabat et al.) of the light and heavy chain variable regions of the anti-CLDN antibodies, SC27.1 (FIG. 2E), SC27.22 (FIG. 2F), SC27.108 (FIG. 2G), and SC27.204 (FIG. 2H), wherein the CDRs are derived using Kabat, Chothia, ABM and Contact methodology. The variable region sequences were analyzed using a proprietary version of the Abysis database to provide the CDR and FR designations. Though the CDRs in FIGS. 2A and 2B are set forth according to Kabat et al., those skilled in the art will appreciate that the CDR and FR designations can also be defined according to Chothia, MacCallum or any other accepted nomenclature system.

The SEQ ID NOS of each particular antibody are sequential odd numbers. Thus the monoclonal anti-CLDN antibody, SC27.1, comprises amino acid SEQ ID NOS: 21 and 23 for the VL and VH, respectively; and SC27.22 comprises SEQ ID NOS: 25 and 27 etc. The corresponding nucleic acid sequence for each antibody amino acid sequence is included in FIG. 2C and has the SEQ ID NO immediately preceding the corresponding amino acid SEQ ID NO. Thus, for example, the SEQ ID NOS of the nucleic acid sequences of the VL and VH of the SC27.1 antibody are SEQ ID NOS: 20 and 22, respectively.

### Example 4

### Generation of Chimeric and Humanized Anti-CLDN Antibodies

Chimeric anti-CLDN antibodies were generated using art-recognized techniques as follows. Total RNA was extracted from the anti-CLDN antibody-producing hybridomas and the RNA was PCR amplified. Data regarding V, D and J gene segments of the VH and VL chains of the mouse antibodies were obtained from the nucleic acid sequences of the anti-CLDN antibodies of the invention (see FIG. 2C for nucleic acid sequences). Primer sets specific to the framework sequence of the VH and VL chain of the antibodies were designed using the following restriction sites: AgeI and XhoI for the VH fragments, and XmaI and DrIII for the VL fragments. PCR products were purified with a Qiaquick PCR purification kit (Qiagen), followed by digestion with restriction enzymes AgeI and XhoI for the VH fragments and XmaI and DraIII for the VL fragments. The VH and VL digested PCR products were purified and ligated into IgH or IgK expression vectors, respectively. Ligation reactions were performed in a total volume of 10 μL with 200 U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto ampicillin plates at a concentration of 100 μg/mL. Following purification and digestion of the amplified ligation products, the VH fragment was cloned into the AgeI-XhoI restriction sites of the pEE6.4 expression vector (Lonza) comprising HuIgG1 (pEE6.4HuIgG1) and the VL fragment was cloned into the XmaI-DraIII restriction sites of the pEE12.4 expression vector (Lonza) comprising a human kappa light constant region (pEE12.4Hu-Kappa).

Chimeric antibodies were expressed by co-transfection of either HEK293T or CHO-S cells with pEE6.4HuIgG1 and pEE12.4Hu-Kappa expression vectors. Prior to transfection the HEK293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 2.5 μg each of pEE6.4HuIgG1 and pEE12.4Hu-Kappa vector DNA were added to 10 μL HEK293T transfection reagent in 1.5 mL Opti-MEM. The mix was incubated for 30 min. at room temperature and added to cells. Supernatants were harvested three to six days after transfection. For CHO-S cells, 2.5 μg each of pEE6.4HuIgG1 and pEE12.4Hu-Kappa vector DNA were added to 15 μg PEI transfection reagent in 400 μL Opti-MEM. The mix was incubated for 10 min. at room temperature and added to cells. Supernatants were harvested three to six days after transfection. Culture supernatants containing recombinant chimeric antibodies were cleared from cell debris by centrifugation at 800×g for 10 min. and stored at 4° C. Recombinant chimeric antibodies were purified with Protein A beads.

Murine anti-CLDN antibodies were humanized using a proprietary computer-aided CDR-grafting method (Abysis Database, UCL Business) and standard molecular engineering techniques as follows. Human framework regions of the variable regions were designed based on the highest homology between the framework sequences and CDR canonical structures of human germline antibody sequences, and the framework sequences and CDRs of the relevant mouse antibodies. For the purpose of the analysis the assignment of amino acids to each of the CDR domains was done in accordance with Kabat numbering. Once the variable regions were selected, they were generated from synthetic gene segments (Integrated DNA Technologies). In some cases, the variable regions were codon optimized and generated by DNA 2.0 (Menlo Park, Calif.). Humanized antibodies were cloned and expressed using the molecular methods described above for chimeric antibodies.

The VL and VH amino acid sequences of the humanized antibodies were derived from the VL and VH sequences of the corresponding mouse antibody (e.g. hSC27.1 is derived from murine SC27.1). There were no framework changes or back mutations made in the light or heavy chain variable regions of the humanized antibodies hSC27.1, hSC27.22 or hSC17.108. However, as shown in Table 5 below two residue changes were made in the heavy chain framework of humanized constructs derived from SC27.204 (i.e., hSC27.204 and hSC27.204v2).

In addition to the framework changes a variant of hSC27.204 was generated to increase molecular stability. The variant antibody, termed hSC27.204v2, shares the same light chain as hSC27.204 (SEQ ID NO: 73) but differs in the heavy chain. More specifically, the heavy chain variable region of hSC27.204v2 (SEQ ID NO: 77) includes a conservative mutation, N58Q, in CDRH2 (SEQ ID NO: 115) of the hSC27.204 heavy chain variable region (SEQ ID NO: 75). This residue position is underlined in FIG. 2B for the hSC27.204 VH sequence (SEQ ID NO: 75) and hSC27.204v2 VH sequence (SEQ ID NO: 77).

Besides the aforementioned humanized constructs, site-specific variants of hSC27.22, hSC27.108 and hSC27.204v2 were constructed (termed hSC27.22ss1, hSC27.108ss1 and hSC27.204v2ss1) for use in accordance with the teachings herein. These site-specific variants are described in more detail in Example 5 below.

Table 5 below shows a summary of the humanized anti CLDN antibodies and their variants, numbered according to Kabat et al. In each case, the binding affinity of the humanized antibody was checked to ensure that it was substantially equivalent to the corresponding mouse antibody. FIG. 2A depicts the contiguous amino acid sequences of the VL of exemplary humanized antibodies and their variants. FIG. 2B depicts the contiguous amino acid sequences of the VH of exemplary humanized antibodies and their variants. The nucleic acid sequences of the light and heavy chain variable regions of the anti-CLDN humanized antibodies are provided in FIG. 2C.

TABLE 5

| | | | human | VH FR | VH CDR | human | human | VK FR | VK CDR |
|---|---|---|---|---|---|---|---|---|---|
| mAb | Isotype | human VH | JH | changes | Changes | VK | JK | changes | Changes |
| hSC27.1 | IgG1/κ | IGHV1-3*01 | JH1 | None | None | IGKV1-12*01 | JK2 | None | None |
| hSC27.22 | IgG1/κ | IGHV1-3*01 | JH6 | None | None | IGKV4-1*01 | JK2 | None | None |
| hSC27.22ss1 | IgG1 | IGHV1-8*01 | JH6 | None | None | IGKV4-1*01 | JK2 | None | None |
| | C220S/κ | | | | | | | | |
| hSC27.108 | IgG1/κ | IGHV1-18*01 | JH1 | None | None | IGKV3-11*01 | JK4 | None | None |
| hSC27.108ss1 | IgG1 | IGHV1-18*01 | JH1 | None | None | IGKV3-11*01 | JK4 | None | None |
| | C220S/κ | | | | | | | | |
| hSC27.204 | IgG1/κ | IGHV3-23*01 | JH1 | A93T K94G | None | IGKV1-16*01 | JK4 | None | None |
| hSC27.204 v2 | IgG1/κ | IGHV3-23*01 | JH1 | A93T K94G | N58Q | IGKV1-16*01 | JK4 | None | None |
| hSC27.204 v2ss1 | IgG1 | IGHV3-23*01 | JH1 | A93T K94G | N58Q | IGKV1-16*01 | JK4 | None | None |
| | C220S/κ | | | | | | | | |

### Example 5

### Generation of Site-Specific ANTI-CLDN Antibodies

Engineered human IgG1/kappa anti-CLDN site-specific antibodies were constructed comprising a native light chain (LC) constant region and mutated heavy chain (HC) constant region, wherein cysteine 220 (C220) in the upper hinge region of the HC, which forms an interchain disulfide bond with cysteine 214 (C214) in the LC, was substituted with serine (C220S). When assembled, the HCs and LCs form an antibody comprising two free cysteines that are suitable for conjugation to a therapeutic agent. Unless otherwise noted, all numbering of constant region residues is in accordance with the EU numbering scheme as set forth in Kabat et al.

The VH nucleic acids were cloned onto an expression vector containing the C220S mutation in the constant region of the HC. The vector encoding the mutant C220S HC of hSC27.22, hSC27.108 or hSC27.204v2 was co-transfected in CHO-S cells with a vector encoding the native IgG1 kappa LC of hSC27.22, hSC27.108 or hSC27.204, and expressed using a mammalian transient expression system. The engineered anti-CLDN site-specific antibody containing the C220S mutant was termed hSC27.22ss1, hSC27.108ss1 or hSC27.204v2ss1, respectively.

The amino acid sequences of the full length heavy chains of the hSC27.22ss1, hSC27.108ss1, and hSC27.204v2ss1 site specific antibodies are shown in FIG. 2D (SEQ ID NOS: 82, 85 and 89, respectively). The amino acid sequence of the LC of hSC27.22ss1 is identical to that of hSC27.22 (SEQ ID NO: 80), the amino acid sequence of the LC of hSC27.108ss1 is identical to that of hSC27.108 (SEQ ID NO: 83) and the amino acid sequence of the LC of hSC27.204v2ss1 is identical to that of the hSC27.204 and hSC27.204v2 antibodies (SEQ ID NO: 86). The site-specific antibodies thus comprise, respectively, light and heavy chains as set forth in SEQ ID NO: 80 and SEQ ID NO: 82 (hSC27.22ss1), SEQ ID NO: 83 and SEQ ID NO: 85 (hSC27.108ss1) and SEQ ID NO: 86 and SEQ ID NO: 89 (hSC27.204v2ss1).

The engineered anti-CLDN site specific antibodies were characterized by SDS-PAGE to confirm that the correct mutants had been generated. SDS-PAGE was conducted on a pre-cast 10% Tris-Glycine mini gel from Life Technologies in the presence and absence of a reducing agent such as DTT (dithiothreitol). Following electrophoresis, the gels were stained with a colloidal coomassie solution (data not shown). Under reducing conditions, two bands corresponding to the free LCs and free HCs, were observed. This pattern is typical of IgG molecules in reducing conditions. Under non-reducing conditions, the band patterns were different from native IgG molecules, indicative of the absence of a disulfide bond between the HC and LC. A band around 98 kD corresponding to the HC-HC dimer was observed. In addition, a faint band corresponding to the free LC and a predominant band around 48 kD that corresponded to a LC-LC dimer was observed. The formation of some amount of LC-LC species is expected due to the free cysteines on the c-terminus of each LC.

### Example 6

### Preparation of Anti-CLDN6 Antibody-Drug Conjugates

Four murine anti-CLDN antibodies (SC27.22, SC27.103, SC27.105 and SC27.108) and three humanized site-specific anti-CLDN antibodies (hSC27.22551, hSC27.108ss1 and hSC27.204v2ss1) were conjugated to a pyrrolobenzodiazepine (PBD1 in the form of DL6) via a terminal maleimido moiety with a free sulfhydryl group to create antibody drug conjugates (ADCs) termed SC27.22PBD1, SC27.103PBD1, SC27.105PBD1, SC27.108PBD1, hSC27.22ss1 PBD1, hSC27.108ss1 PBD1 and hSC27.204v2ss1 PBD1.

The murine anti-CLDN ADCs were prepared as follows. The cysteine bonds of anti-CLDN antibodies were partially reduced with a pre-determined molar addition of mol tris(2-carboxyethyl)-phosphine (TCEP) per mol antibody for 90 min. at room temperature in phosphate buffered saline (PBS) with 5 mM EDTA. The resulting partially reduced preparations were then conjugated to PBD1 (the structure of PBD1 is provided above in the current specification) via a maleimide linker for a minimum of 30 mins. at room temperature. The reaction was then quenched with the addition of excess N-acetyl cysteine (NAC) compared to linker-drug using a 10 mM stock solution prepared in water. After a minimum quench time of 20 mins, the pH was adjusted to 6.0 with the addition of 0.5 M acetic acid. The preparations of the ADCs were buffer exchanged into diafiltration buffer by diafiltration using a 30 kDa membrane. The dialfiltered anti-CLDN ADCs were then formulated with sucrose and polysorbate-20 to the target final concentration. The resulting anti-CLDN ADCs were analyzed for protein concentration (by measuring UV), aggregation (SEC), drug to antibody ratio (DAR) by reverse-phase HPLC (RP-HPLC) and activity (in vitro cytotoxicity).

The site specific humanized anti-CLDN ADCs were conjugated using a modified partial reduction process. The desired product is an ADC that is maximally conjugated on the unpaired cysteine (C214) on each LC constant region and that minimizes ADCs having a drug to antibody ratio (DAR) which is greater than 2 (DAR>2) while maximizing ADCs having a DAR of 2 (DAR=2). In order to further improve the specificity of the conjugation, the antibodies were selectively reduced using a process comprising a stabilizing agent (e.g. L-arginine) and a mild reducing agent (e.g. glutathione) prior to conjugation with the linker-drug, followed by a diafiltration and formulation step.

A preparation of each antibody was partially reduced in a buffer containing 1M L-arginine/5 mM EDTA with a pre-determined concentration of reduced glutathione (GSH), pH 8.0 for a minimum of two hours at room temperature. All preparations were then buffer exchanged into a 20 mM Tris/3.2 mM EDTA, pH 7.0 buffer using a 30 kDa membrane (Millipore Amicon Ultra) to remove the reducing buffer. The resulting partially reduced preparations were then conjugated to PBD1 (the structure of PBD1 is provided above in the current specification) via a maleimide linker for a minimum of 30 mins. at room temperature. The reaction was then quenched with the addition of excess NAC compared to linker-drug using a 10 mM stock solution prepared in water. After a minimum quench time of 20 minutes, the pH was adjusted to 6.0 with the addition of 0.5 M acetic acid. The preparations of the ADCs were buffer exchanged into diafiltration buffer by diafiltration using a 30 kDa membrane. The dialfiltered anti-CLDN ADC was then formulated with sucrose and polysorbate-20 to the target final concentration. The resulting anti-CLDN ADCs were analyzed for protein concentration (by measuring UV), aggregation (SEC), drug to antibody ratio (DAR) by reverse-phase HPLC (RP-HPLC) and activity (in vitro cytotoxicity).

### Example 7

### Characteristics of Anti-CLDN Antibodies and ADCs

Various methods were used to characterize the anti-CLDN antibodies generated in Examples 2 and 4 in terms of isotype, affinity and cross reactivity with other CLDN family members.

The murine antibodies generated as described in Example 2, were characterized to determine whether they cross reacted with CLDN family members using flow cytometry analyses were performed as follows: HEK293T cells were stably transduced with lentiviral vectors encoding hCLDN6, hCLDN9, or hCLDN4 as described in Example 1. 1×10⁵ HEK293T cells stably transduced with the aforementioned expression constructs were incubated at 4° C. for 30 mins. with anti-CLDN antibodies, diluted to 10 μg/ml into a final volume of 50 μl PBS/2% FCS. Following incubation, cells were washed with 200 μL PBS/2% FCS, pelleted by centrifugation, supernatant was discarded, and cell pellets were resuspended in 50 μL per sample DyeLight 649 labeled goat-anti-mouse IgG, Fc fragment specific secondary antibody diluted 1:200 in PBS/2% FCS. After a 15 min. incubation at 4° C. cells were washed and pelleted twice with PBS/2% FCS as previously described and resuspended in 100 μL PBS/2% FCS with 2 μg/mL 4′,6-diamidino-2-phenylindole dihydrochloride (DAPI). Samples were analyzed by flow cytometry and live cells were assessed with DyeLight 649 for fluorescence exceeding that of cells stained with an isotype control antibody.

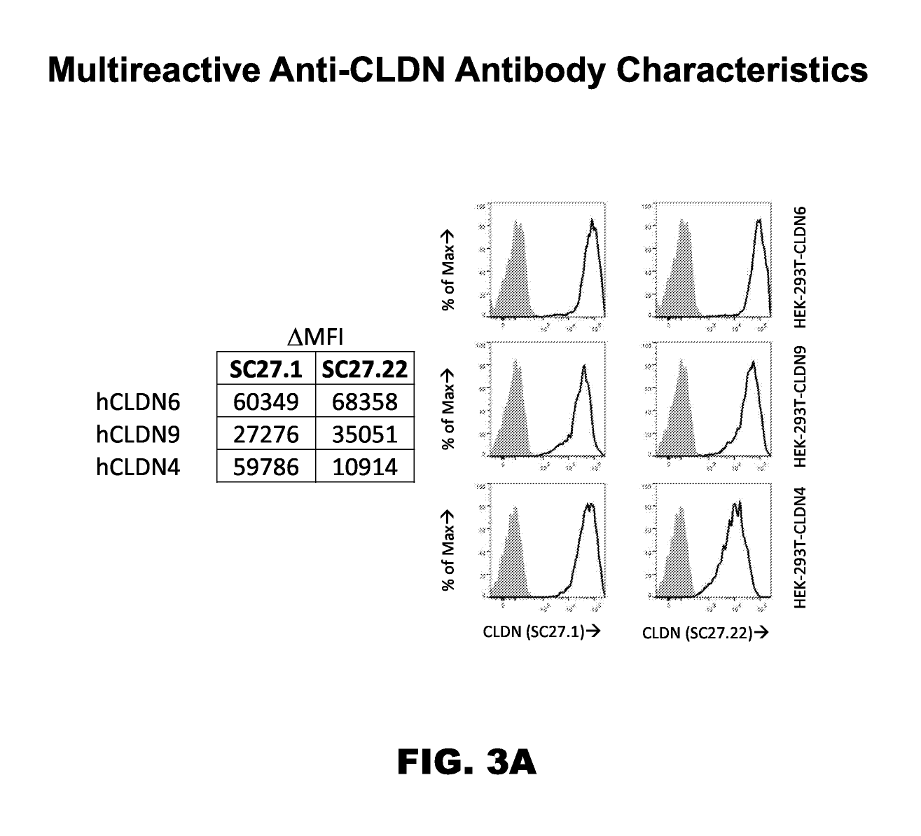

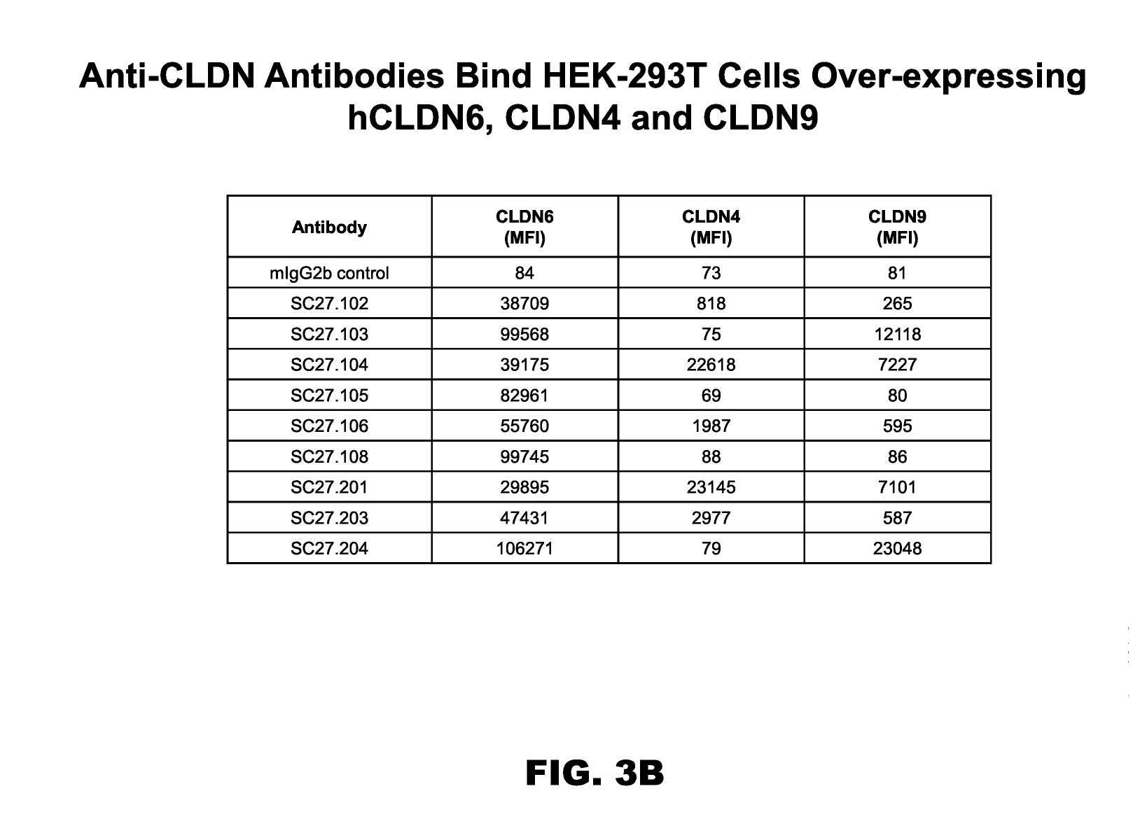

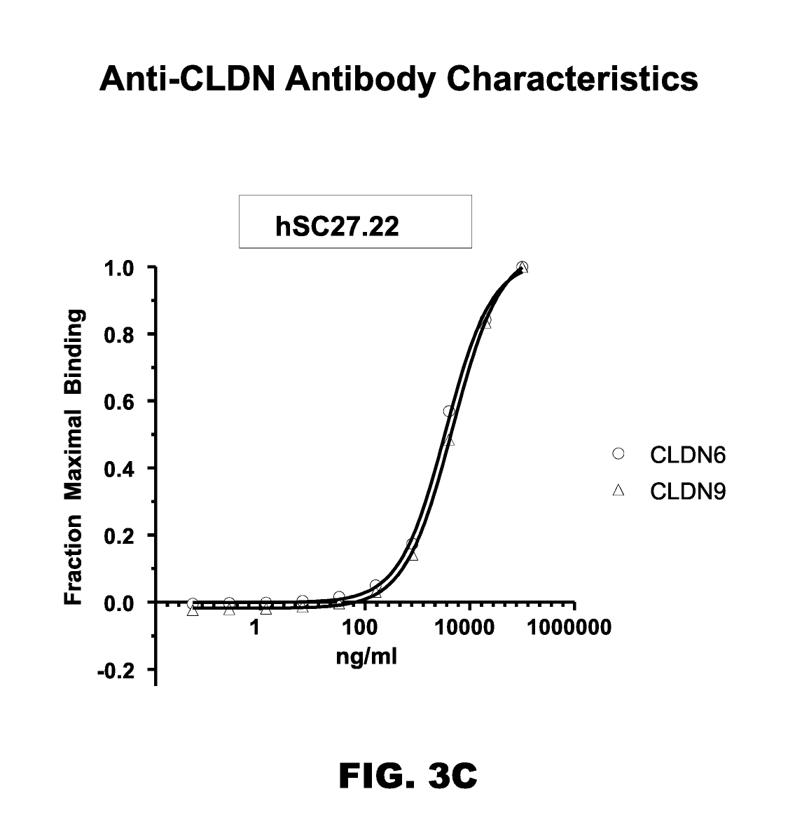

The flow cytometry assay described above resulted in the identification of numerous anti-CLDN antibodies. Cross reactivity was determined based on the change in geometric mean fluorescence intensity (ΔMFI) for the binding of the antibody to the cell lines specifically overexpressing the indicated CLDN family member versus the signal determined using a fluorescence minus one (FMO) isotype-control (gray-fill) (FIG. 3A). Thus, the two hCLDN6-binding antibodies SC27.1 and SC27.22 can be described as claudin multireactive antibodies since they cross react in this assay with three members of the human CLDN family: hCLDN6, hCLDN4 and hCLDN9. SC27.1 and SC27.22 antibodies also bound to mouse and rat orthologs of CLDN4 and CLDN9 (data not shown).

To test the ability of various additional mouse antibodies to bind to CLDN family members, flow cytometry was performed using cell lines overexpressing human CLDN4, CLDN6 or CLND9 that had been incubated with 10 μg/mL of purified primary anti-CLDN antibody, or a mouse IgG2b control antibody, followed by incubation with an Alexa 647 anti-mouse secondary antibody. As shown in FIG. 3B, all the antibodies bound to CLDN6, whereas some were CLDN6-specific (e.g. SC27.102, SC27.105, and SC27.108), and others were multireactive and bound to both CLDN6 and CLDN9 (e.g., SC27.103 and SC27.204), or to CLDN6 and CLDN4 (e.g., SC27.104). Thus a wide range of multireactive binding profiles was obtained for the antibodies of the invention.

To compare the apparent binding affinity of the multireactive anti-CLDN antibodies for CLDN6 and CLDN9, flow cytometry was performed with a serial dilution of humanized anti-CLDN antibody hSC27.22. The antibody was serially diluted to concentrations ranging from 50 μg/ml to 100 μg/ml and was added to a 96 well plate containing HEK293T cells overexpressing CLDN6 or CLDN9, and kept on ice for one hour. A secondary anti-human antibody (Jackson ImmunoResearch Cat. #109-605-098) was added and incubated for one hour in the dark. The cells were washed twice in PBS after which Fixable Viability Dye (eBioscience Cat #65-0863-14) was added for 10 mins. Following additional washing with PBS, cells were fixed with paraformaldehyde (PFA) and read on a BD FACS Canto II flow cytometer in accordance with the manufacturer's instructions. MFI values were normalized using fluorescent microspheres (Bangs Laboratories) according to manufacturer's instructions. Normalized maximal MFI values observed for the binding of the antibody to either CLDN6 or CLDN9 expressing cells were used to transform the data into fraction maximal binding for each overexpressing cell, using the equation: fraction maximal binding=(observed normalized MFI/maximal normalized MFI). Apparent EC50 values for the binding of hSC27.22 to each cell line were then calculated using a four parameter variable slope curve fitting for a log (inhibitor) vs. response model supplied in the Graph Pad Prism software package (La Jolla, Calif.). FIG. 3C shows that the humanized multireactive anti-CLDN6 antibody, hSC27.22, has an apparent EC50 for CLDN6 which is substantially the same as that for CLDN9. (apparent EC50 CLDN6—3.45 μg/mL (r² for goodness of fit=0.9987, 99% confidence bounds: 2.51-4.75 μg/mL); apparent EC50 CLDN9-4.66 μg/mL (r² for goodness of fit=0.9998, 99% confidence bounds: 4.09-5.31 μg/mL)).

### Example 8

### Anti-CLDN Antibodies Facilitate Delivery of Cytotoxic Agents In Vitro

To determine whether anti-CLDN antibodies are able to internalize and mediate the delivery of cytotoxic agents to live tumor cells, an in vitro cell killing assay was performed using selected anti-CLDN antibodies and saporin linked to a secondary anti-mouse antibody FAB fragment. Saporin is a plant toxin that deactivates ribosomes, thereby inhibiting protein synthesis and resulting in the death of the cell. Saporin is only cytotoxic inside the cell where it has access to ribosomes, but is unable to internalize on its own. Therefore, saporin-mediated cellular cytotoxicity in these assays is indicative of the ability of the anti-mouse FAB-saporin conjugate to internalize into the target cell only upon binding and internalization of anti-CLDN antibodies.

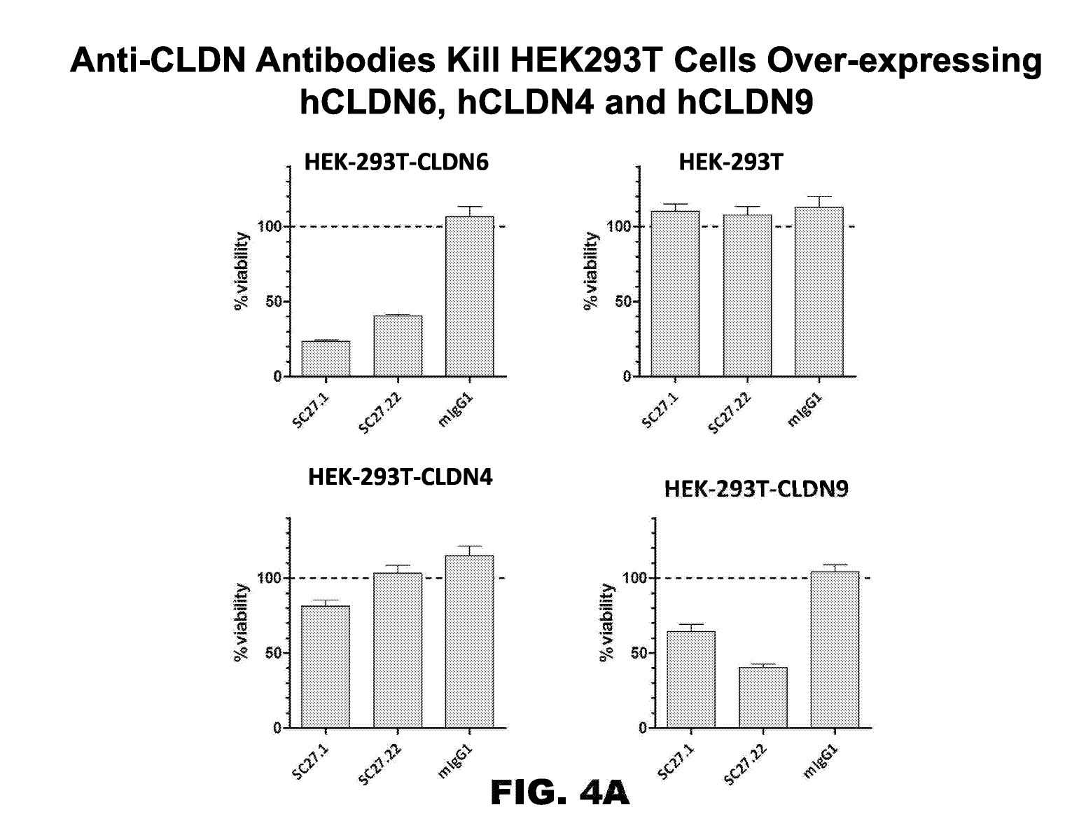

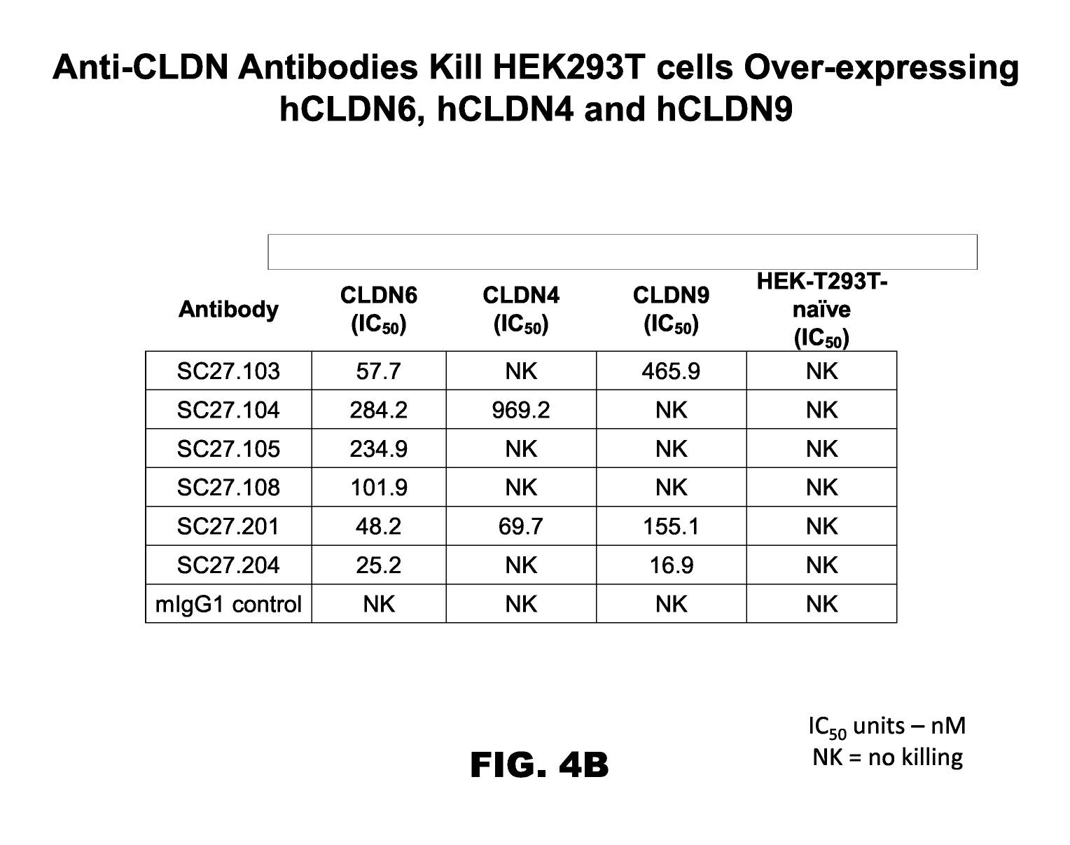

Single cell suspensions of HEK293T cells and HEK293T cells overexpressing hCLDN6, hCLDN4, or hCLDN9 were plated at 500 cells per well into BD Tissue Culture plates (BD Biosciences). One day later, 250 μM of purified SC27.1, SC27.22, or isotype control (mIgG1) antibodies and a fixed concentration of 2 nM anti-Mouse IgG FAB-saporin conjugate (Advanced Targeting Systems) were added to the culture. The HEK293T cells were incubated for 72 hours post antibody treatment. After the incubation, viable cells were enumerated using CellTiter-Glo® (Promega) as per the manufacturer's instructions. Raw luminescence counts using cultures containing cells incubated only with the secondary FAB-saporin conjugate were set as 100% reference values and all other counts calculated accordingly. Both of the anti-CLDN antibodies, SC27.1 and SC27.22, at a concentration of 250 μM effectively killed HEK293T cells overexpressing hCLDN6 and hCLDN9 (FIG. 4A), whereas the mouse IgG1 isotype control antibody (mIgG1) at the same concentration did not. Naïve HEK293T cells were not effectively killed by the treatment whereas HEK293T cells overexpressing hCLDN4 were effectively killed by SC27.1 but were not killed by SC27.22 treatment at the dose tested. The dashed horizontal line represents the level at which no cytotoxicity was observed.

In order to determine the apparent IC50 of additional antibodies for CLDN4, CLDN6 or CLDN9, the experiment described in the paragraph above was repeated with titrations of antibodies, across a concentration range of 0.15 nM to 1000 nM (FIG. 4B). The percentage of cell killing observed at each antibody concentration was enumerated by CellTiter-Glo® as described above, and a curve was fitted to the resulting data in order to calculate an apparent IC50 for the killing activity of antibody on each cell line. Antibodies which had an apparent IC50 of >2000 nM were deemed not to kill a particular cell line and are denoted as “NK” in FIG. 4B. A control mouse IgG1 antibody also did not kill any of the cell lines tested. Although this cytotoxicity assay measures the ability of various antibodies to mediate delivery of a cytotoxin via internalization of bound antigen rather than providing a direct measure of antibody binding affinity, the apparent 1050 of the antibodies shown in FIG. 4B in general correlates well with the single point flow cytometry data presented in FIG. 3B. For example, in both experiments SC27.108 is shown to be CLDN6-specific (apparent IC50=100 nM). Similarly, by flow cytometry SC27.103 shows strong binding to CLDN6 and moderate binding to CLDN9, which correlates with an apparent 1050 value of 58 nM for CLDN6 and 466 nM for CLDN9. However, it is also clear that detectable binding above background does not always result in detectable killing (e.g., SC27.104 binds to CLDN9 (see FIG. 3B) but is not able to effectively internalize and kill CLDN9-overexpressing cells (see FIG. 4B); whereas SC27.201 binds CLDN9 (see FIG. 3B) and is able to internalize into cells expressing CLDN9 and kill those cells (see FIG. 4B)).

Together, the above results demonstrate the ability of multireactive anti-CLDN antibodies to mediate internalization and their ability to deliver cytotoxic payloads, supporting the hypothesis that anti-CLDN antibodies may have therapeutic utility as the targeting moiety for an ADC.

### Example 9

### Humanized Anti-CLDN Antibody Drug Conjugates Suppress Tumor Growth In Vivo

Anti-CLDN ADCs, generated as described in Example 6 above, were tested to demonstrate their ability to suppress OV and LU-Ad tumor growth in immunodeficient mice.

PDX tumor lines expressing CLDN (e.g. OV91, OV78, and LU134), were grown subcutaneously in the flanks of female NOD/SCID mice using art-recognized techniques. Tumor volumes and mouse weights were monitored once or twice per week. When tumor volumes reached 150-250 mm³, mice were randomly assigned to treatment groups. Mice carrying OV91 tumors were injected with a single dose of 2 mg/kg SC27.1.PBD1 or SC27.22.PBD1, or anti-hapten control mouse IgG1 PBD1. Mice carrying OV78 tumors were injected with a single dose of 1.6 mg/kg hSC27.204v2ss1PBD1 or anti-hapten control IgG1PBD1. Mice carrying LU-Ad tumors were injected with a single dose of 2 mg/kg SC27.22.PBD1 or anti-hapten mouse IgG1PBD1 control.

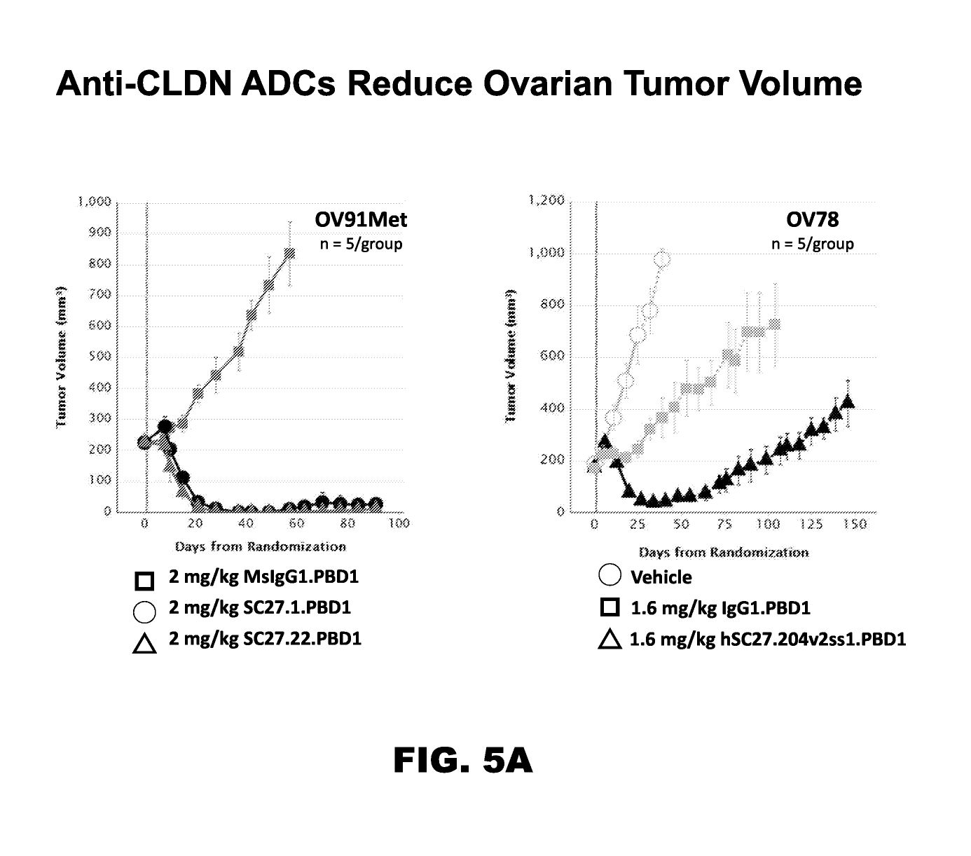

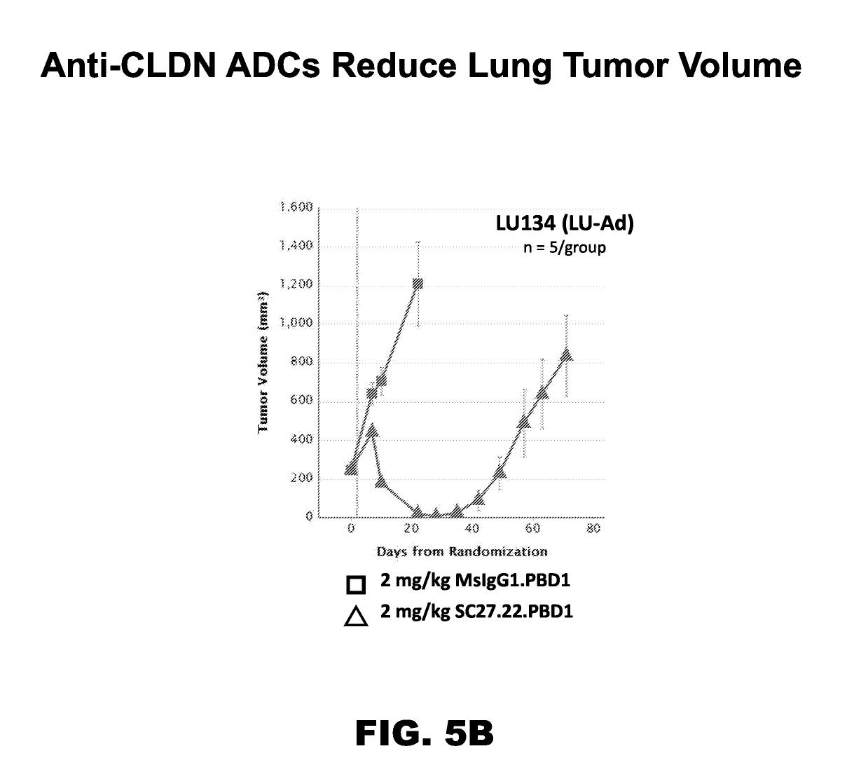

Following treatment, tumor volumes and mouse weights were monitored until tumors exceeded 800 mm³ or mice became sick. Mice treated with anti-CLDN ADCs did not exhibit any adverse health effects beyond those typically seen in immunodeficient, tumor-bearing NOD/SCID mice. The administration of the anti-CLDN ADCs, resulted in significant tumor suppression lasting over 150 days in mice carrying OV91 tumor (FIG. 5A) and over 60 days for mice carrying LU134 tumor (FIG. 5B), whereas the administration of the control ADC IgG1 PBD1 did not result in tumor volume reduction. Administration of the anti-CLDN ADCs in mice carrying OV78 tumors showed a significant delay to tumor progression of about 120 days relative to vehicle and about 90 days relative to isotype control

The ability of anti-CLDN ADCs to specifically kill CLDN-expressing tumor cells and dramatically suppress tumor growth in vivo for extended periods further validates the use of anti-CLDN ADCs in the therapeutic treatment of cancer and in particular in OV and LU cancer.

### Example 10

### Enrichment of CLDN Expression in Cancer Stem Cell Populations

Tumor cells can be divided broadly into two types of cell subpopulations: non-tumorigenic cells (NTG) and tumor initiating cells or tumorigenic cells. Tumorigenic cells have the ability to form tumors when implanted into immunocompromised mice, whereas non-tumorigenic cells do not. Cancer stem cells (CSCs) are a subset of tumorigenic cells and are able to self-replicate indefinitely while maintaining the capacity for multilineage differentiation.

To determine whether the anti-CLDN antibodies of the invention are able to detect tumorigenic CSC populations, PDX tumors were dissociated into single cell suspensions and selective markers, CD46^{hi}CD324⁺, were used to enrich for CSC tumor cell subpopulations (see WO 2012/031280) as follows.

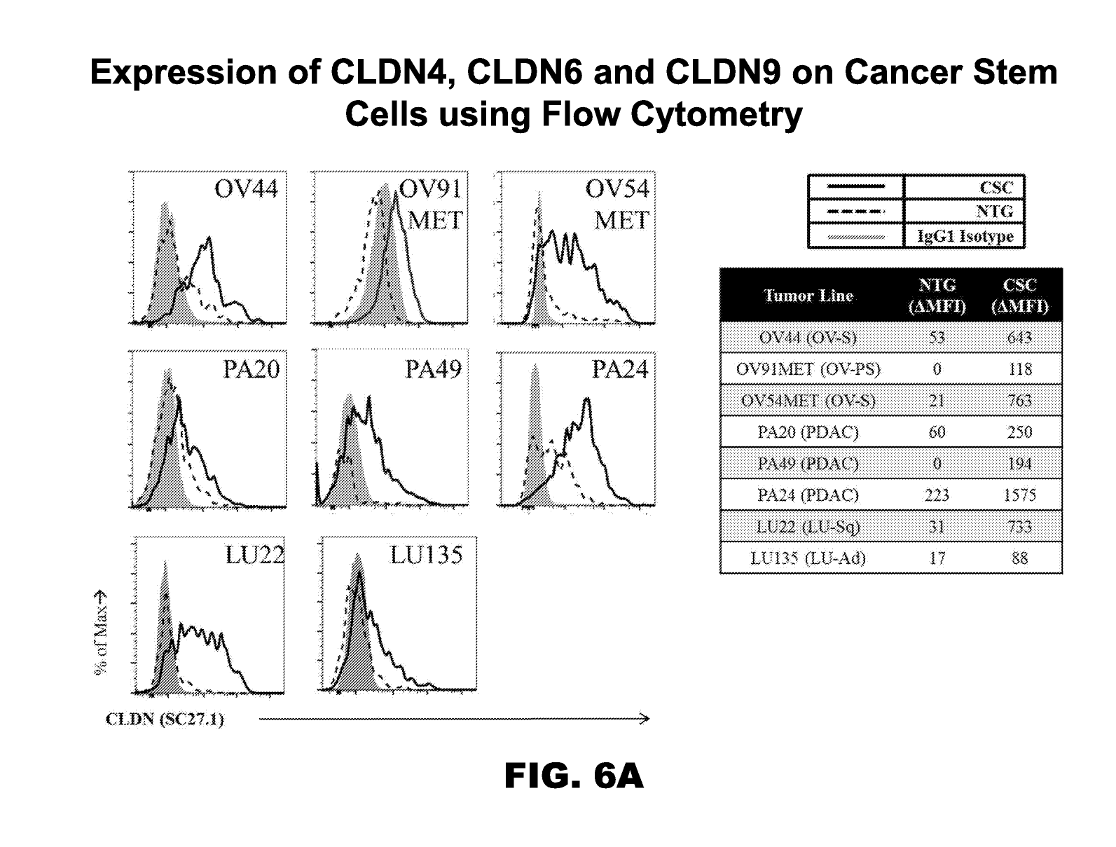

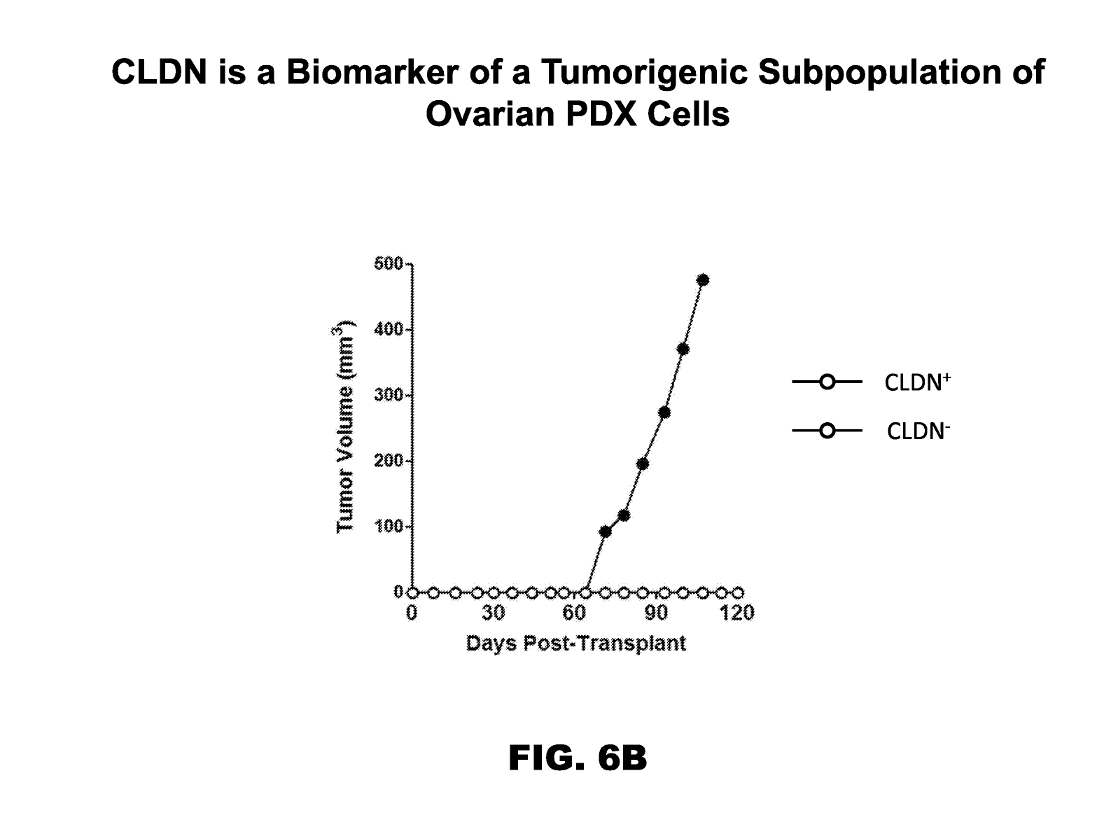

PDX tumor single cell suspensions were incubated with the following antibodies: anti-CLDN SC27.1; anti-human EPCAM; anti-human CD46; anti-human CD324; and anti-mouse CD45 and H-2 kD antibodies. The tumor cells were then assessed for staining by flow cytometry using a BD FACS Canto II flow cytometer. The human EPCAM⁺CD46^{hi}CD324⁺ CSC tumor cell subpopulations of OV-S (e.g., OV44 and OV54MET), OV-PS (e.g. OV91 MET), PA, LU-Ad (e.g., LU135), and LU-Sq (e.g., LU22) PDX tumors demonstrated positive staining with the anti-CLDN SC27.1 antibody, whereas NTG cells (CD46^{lo/-} and/or CD324⁻) demonstrated significantly less staining with anti-CLDN antibodies (FIG. 6A). Isotype control antibodies and FMO controls were employed to confirm staining specificity as is standard practice in the art. A table summarizing the differential staining of anti-CLDN antibodies observed on the surface of CSC and NTG cells is shown in FIG. 6A, with expression enumerated as the change in geometric mean fluorescence intensity (ΔMFI) between the indicated anti-CLDN antibody and the isotype control for the respective tumor cell subpopulations. These data confirm the expression of hCLDN proteins on CSCs.

To determine whether CLDN expression in tumors could be correlated with enhanced tumorigenicity, the following study was conducted. Human OV PDX tumor samples (OV91MET) were grown in immunocompromised mice and were resected after the tumor reached 800-2,000 mm³. The tumors were dissociated into single cell suspensions using art-recognized enzymatic digestion techniques (see, for example, U.S.P.N. 2007/0292414). Human OV PDX tumor cells were stained with mouse anti-CD45 or anti-H2 kD antibodies, and with anti-ESA antibodies to differentiate between human tumor cells and mouse cells. The tumors were also stained with anti-CLDN antibody (SC27.22) and then sorted using a FACSAria™ Flow Cytometer (BD Biosciences). The human OV PDX tumor cells were separated into CLDN⁺ and CLDN⁻ subpopulations. Five female NOD/SCID immunocompromised mice were injected subcutaneously with 200 CLDN⁺ OV tumor cells; and five mice were injected with 200 CLDN⁻ OV tumor cells. Tumor volumes were measured on a weekly basis for four months.

FIG. 6B shows that CLDN⁺ (closed circles) tumor cells were able to functionally reconstitute tumors in vivo, whereas CLDN⁻ tumors (open circles) were not. Thus, tumor cells expressing CLDN were much more tumorigenic than those tumor cells that did not express CLDN, suggesting that the CLDN protein can functionally define a tumorigenic subpopulation within human tumors, and supporting the concept that selected anti-CLDN ADCs can be used to target a tumorigenic subpopulation of tumor cells, which could result in significant tumor regression and prevention of tumor recurrence.

### Example 11

### Reduction of Cancer Stem Cell Frequency by anti-CLDN Antibody-Drug Conjugates

As demonstrated in Example 10, CLDN expression is associated with cancer stem cells. Accordingly, to demonstrate that treatment with anti-CLDN ADCs reduces the frequency of cancer stem cells (CSC) that are known to be drug resistant and to fuel tumor recurrence and metastasis, in vivo limiting dilution assays (LDA) were performed as described below.

LU187 tumors were grown subcutaneously in immunodeficient mice. When tumor volumes averaged 150 mm³-250 mm³ in size, the mice were randomly segregated into two groups. One group was injected intraperitoneally with a human IgG1 conjugated to a drug as a negative control; and the other group was injected with 2 mg/kg anti-CLDN SC27.22PBD1 or anti-hapten mouse IgG1PBD1 control. One week following dosing, two representative mice from each group were euthanized and their tumors were harvested and dispersed to single-cell suspensions. The tumor cells from each treatment group were then harvested, pooled and disaggregated. The cells were labeled with FITC conjugated anti-mouse H2 kD and anti-mouse CD45 antibodies to detect mouse cells; EpCAM to detect human cells; and DAPI to detect dead cells. The resulting suspension was then sorted by FACS using a BD FACS Canto II flow cytometer and live human tumor cells were isolated and collected.

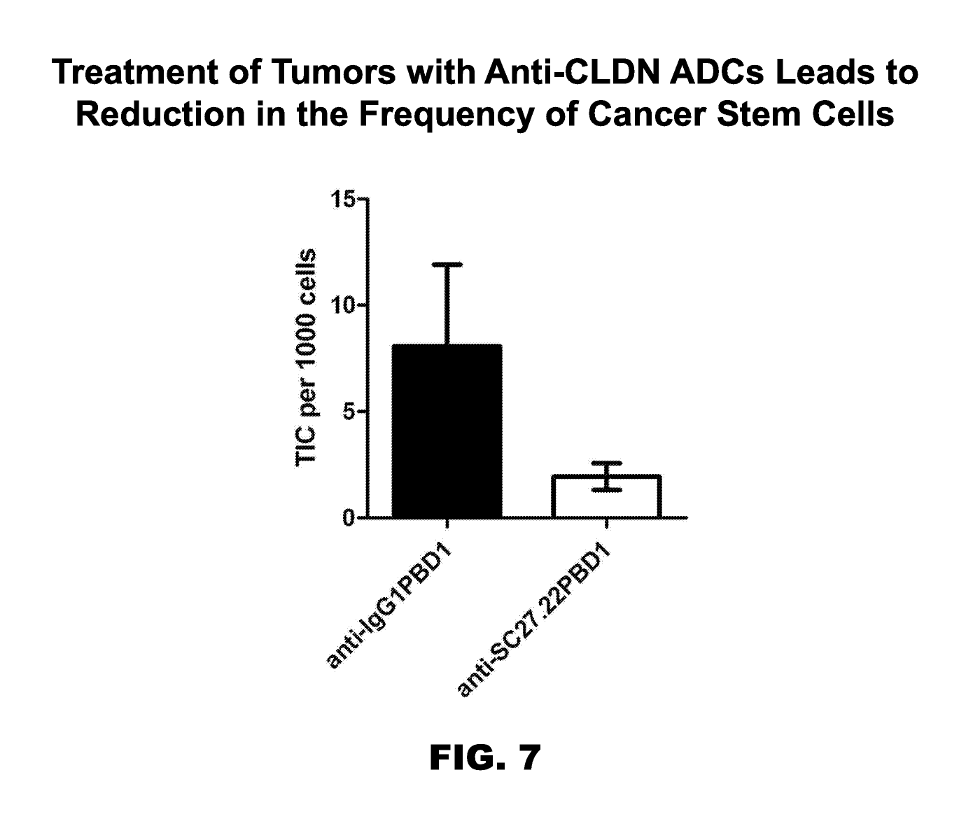

Four cohorts of mice were injected with either 1250, 375, 115 or 35 sorted live, human cells from tumors treated with anti-CLDN ADC. As a negative control four cohorts of mice are transplanted with either 1000, 300, 100 or 30 sorted live, human cells from tumors treated with the control IgG1 ADC. Tumors in recipient mice were measured weekly, and individual mice were euthanized before tumors reached 1500 mm³. Recipient mice were scored as having positive or negative tumor growth. Positive tumor growth was defined as growth of a tumor exceeding 100 mm³. Poisson distribution statistics (L-Calc software, Stemcell Technologies) was used to calculate the frequency of CSCs in each population. As can be seen in FIG. 7, CLDN is associated with tumor initiating cells; tumors treated with anti-CLDN ADC, SC27.22PBD1 showed a reduction in tumor initiating cells of approximately 4-fold compared to tumors treated with control ADC.

### Example 12

### CLDN Expression Profiles in Primary Tumors from the Cancer Genome Atlas

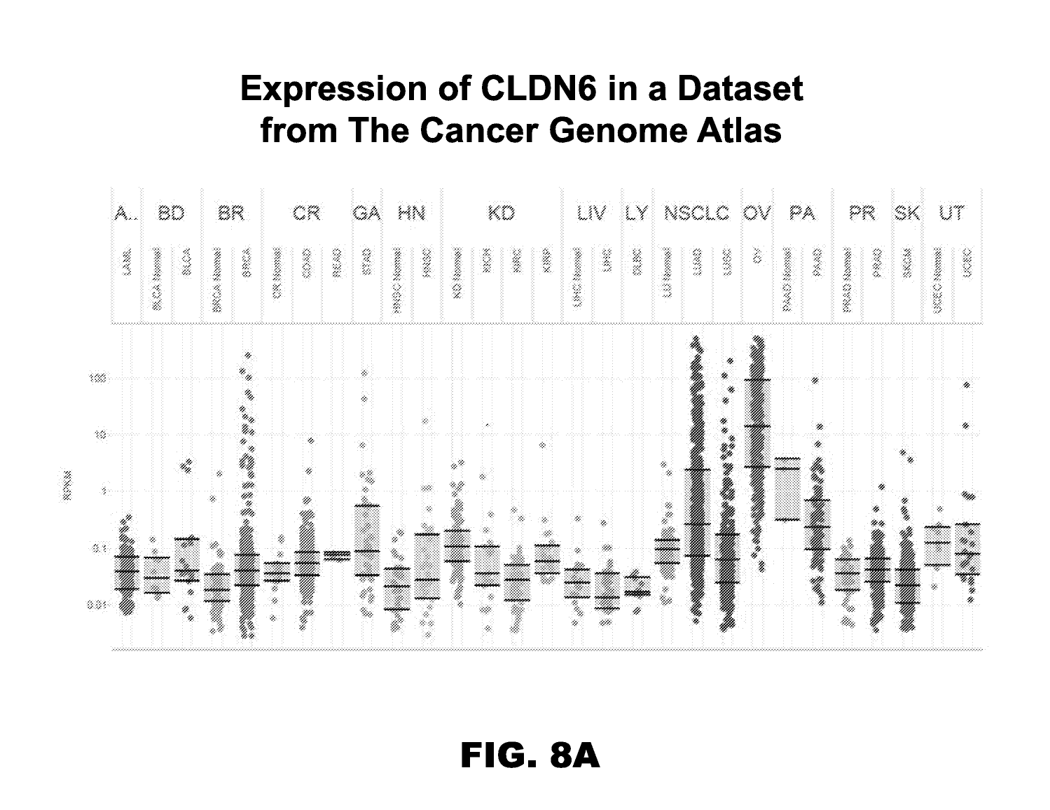

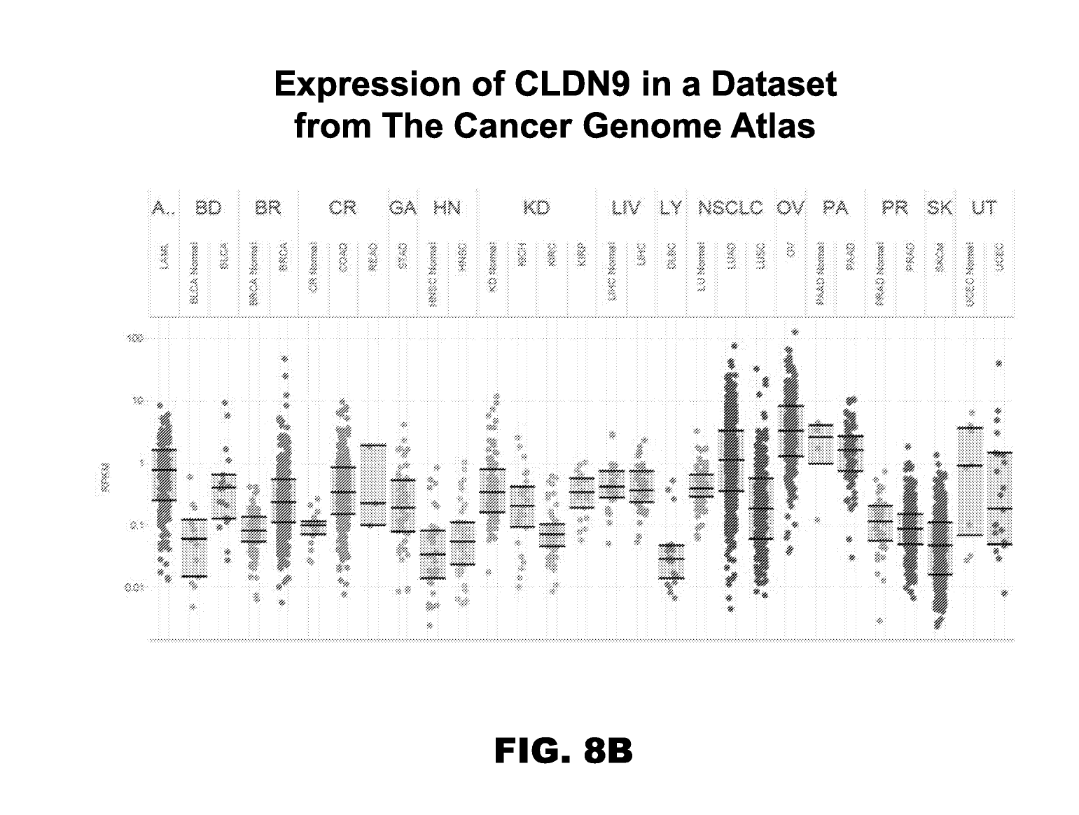

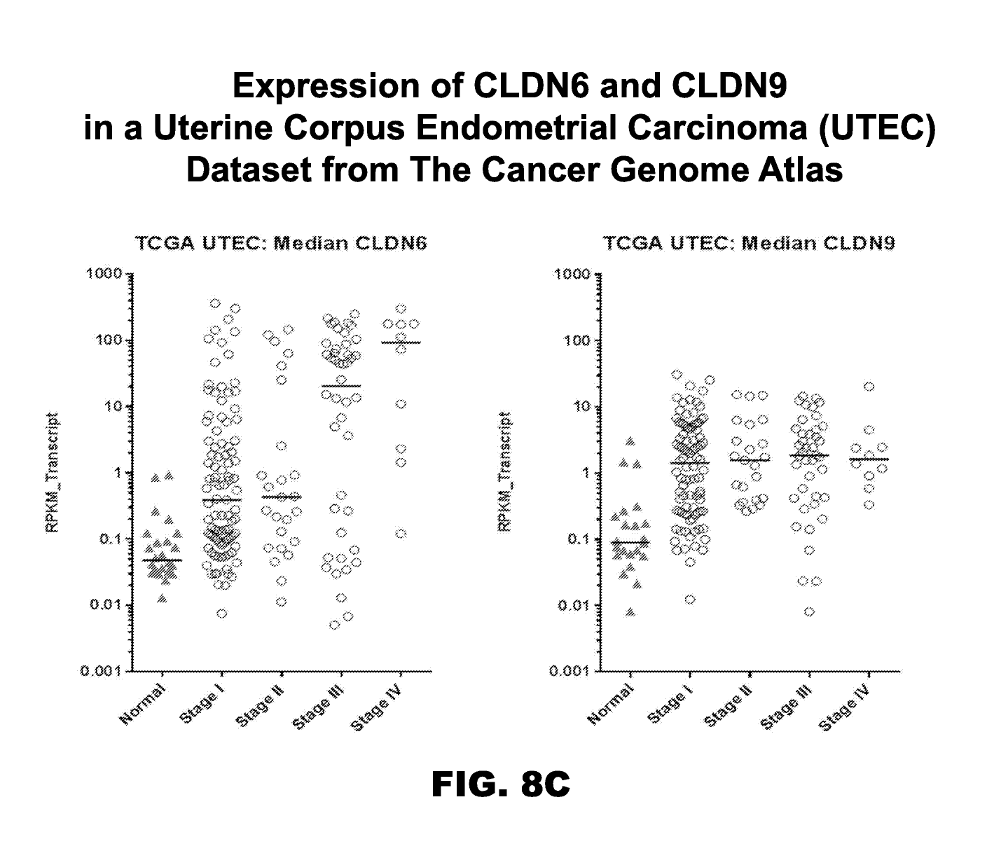

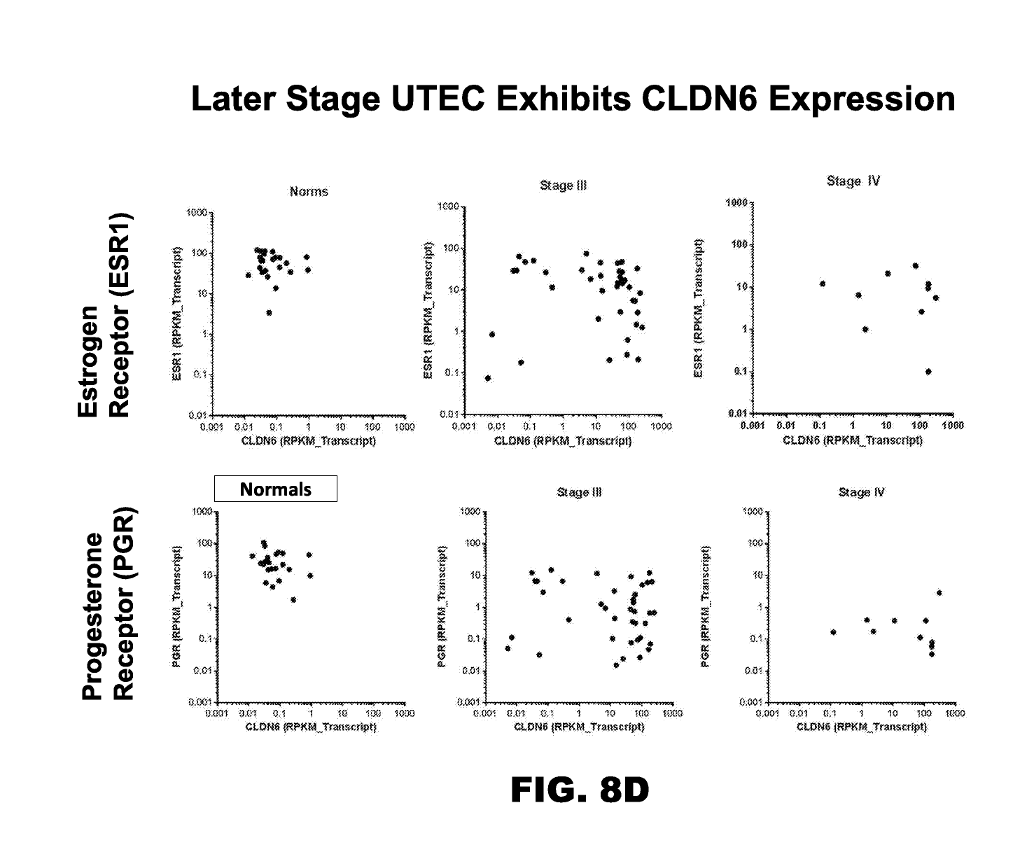

Overexpression of mRNA of CLDN6 and CLDN9 family members was confirmed in various tumors using a large, publically available dataset of tumors and normal samples known as The Cancer Genome Atlas (TCGA, National Cancer Institute). Exon level 3 expression data from the IlluminaHiSeq_RNASeqV2 platform was downloaded from the TCGA Data Portal (https://tcga-data.nci.nih.gov/tcga/tcgaDownload.jsp) and parsed to aggregate the reads from the individual exons of each single gene to generate a single value read per kilobase of exon per million mapped reads (RPKM) for each gene in each sample. The rolled up data was then displayed using Tableau software. The parsed data for CLDN6 and CLDN9 are shown in FIGS. 8A and 8B, respectively, in which each sample is represented as a single dot, and the black horizontal lines represent the quartile boundaries for the setoff data points within a given normal tissue or tumor subtype. FIG. 8A shows that CLDN6 expression is elevated in OV tumors, which were subtyped as ovarian serous cystadenocarcinomas, compared to all other normal tissues. In addition, CLDN6 is elevated in a large number of LU-Ad samples compared to normal lung samples, and a substantial number of breast invasive carcinoma tumors (BRCA). Similar overexpression patterns can be see for CLDN9 as those observed for CLDN6 (FIG. 8B). Again, these data indicate that CLDN6 and CLDN9 expression levels are indicative of tumorigenesis in various tumors and reinforce their selection as potential therapeutic targets.

Overexpression of mRNA of CLDN6 can also be seen in a subset of uterine corpus endometrial carcinomas (UTEC) contained within the TOGA dataset (FIG. 8C). While both CLDN6 and CLDN9 show elevated expression in tumor samples relative to normal uterine tissue, CLDN6 clearly showed progressive elevation in later stage UTECs. Additionally, CLDN6 expression appears to be elevated in the same late stage tumors that lose progesterone receptor expression and therefore may be unresponsive to hormone therapy (FIG. 8D). Together these data indicate that ovarian, uterine endometrial, non-small cell lung carcinomas (both adenocarcinomas and squamous subtypes), and breast carcinomas may be suitable indications for application of antibody drugs targeted to CLDN proteins.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1-30. (canceled)

31. A monoclonal antibody comprising a light chain comprising an amino acid sequence set forth as SEQ ID NO: 86 and a heavy chain comprising an amino acid sequence set forth as SEQ ID NO: 89.

32. The monoclonal antibody of claim 31, wherein the monoclonal antibody is conjugated to a cytotoxic agent.

33. The monoclonal antibody of claim 32, wherein the cytotoxic agent is a pyrrolobenzodiazepine having the structure:

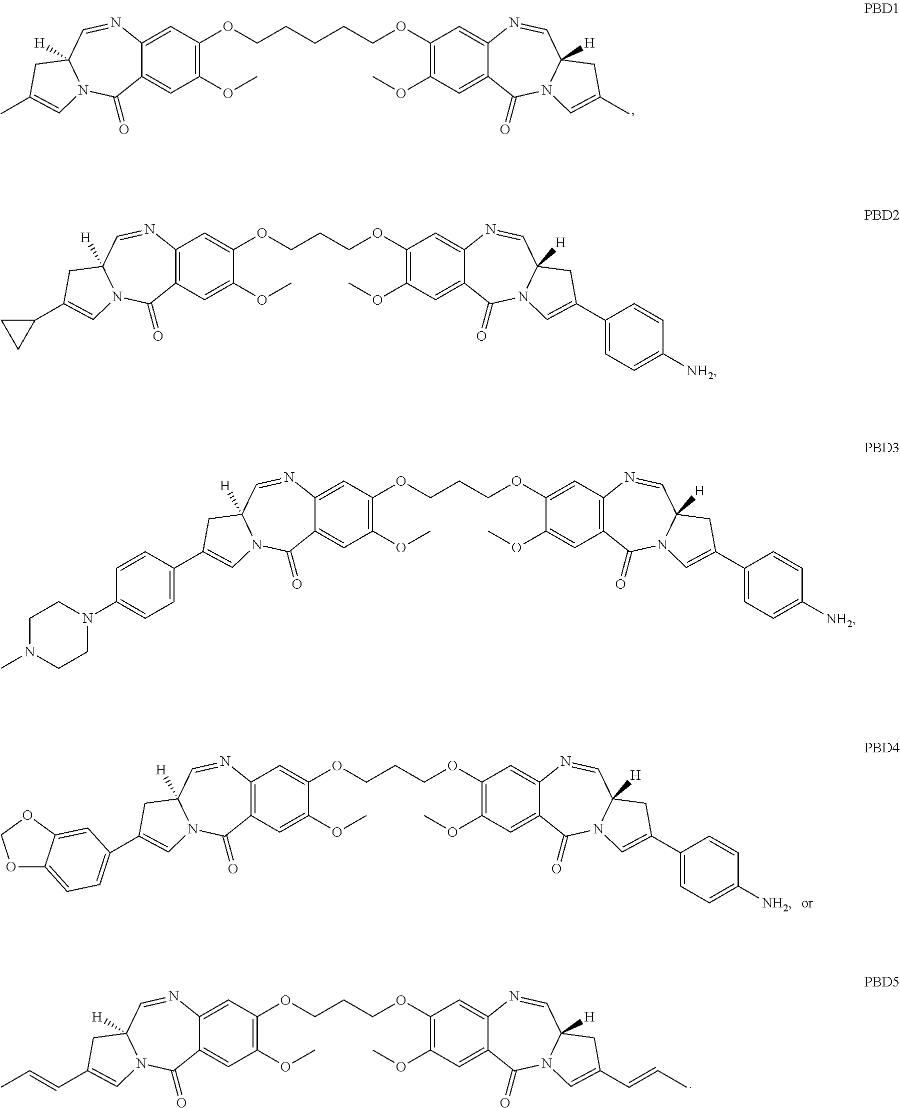

34. An isolated nucleic acid encoding a heavy chain or a light chain of the monoclonal antibody of claim 31.

35. A vector or host cell comprising the isolated nucleic acid of claim 34.

36. An antibody drug conjugate comprising a monoclonal antibody conjugated, linked, or otherwise associated with a cytotoxic agent;
wherein the monoclonal antibody binds to a human CLDN6 protein; and
wherein the cytotoxic agent is a pyrrolobenzodiazepine having the structure:

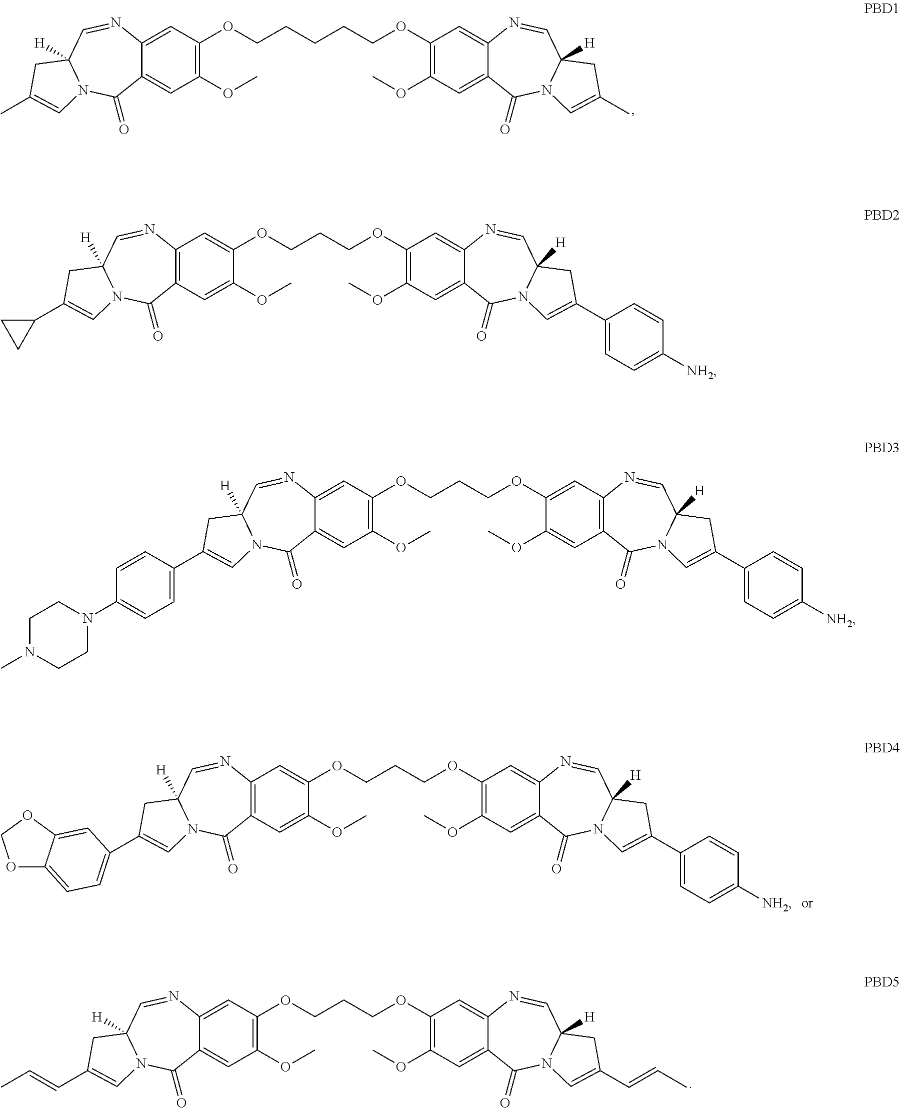

37. The antibody drug conjugate of claim 36, wherein the monoclonal antibody comprises a light chain variable region comprising an amino acid sequence set forth as SEQ ID NO: 73 and a heavy chain variable region comprising an amino acid sequence set forth as SEQ ID NO: 77.

38. The antibody drug conjugate of claim 36, wherein the monoclonal antibody comprises a light chain comprising an amino acid sequence set forth as SEQ ID NO: 86 and a heavy chain comprising an amino acid sequence set forth as SEQ ID NO: 89.

39. The antibody drug conjugate of claim 36, wherein the linker comprises a cleavable linker.

40. The antibody drug conjugate of claim 36, comprising the structure: 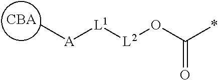
wherein:
CBA is a cell binding agent, which is the monoclonal antibody;
A, L¹, and L² are components of the linker L;
A is a group connecting L¹ to the cell binding agent (CBA);
L¹ is a cleavable linker;
L² is a covalent bond or together with the —OC(═O)— group forms a self-immolative linker; and
wherein the linker L is attached to the cytotoxic agent at the position of the asterisk (*).

41. The antibody drug conjugate of claim 36, wherein the antibody drug conjugate has the structure: 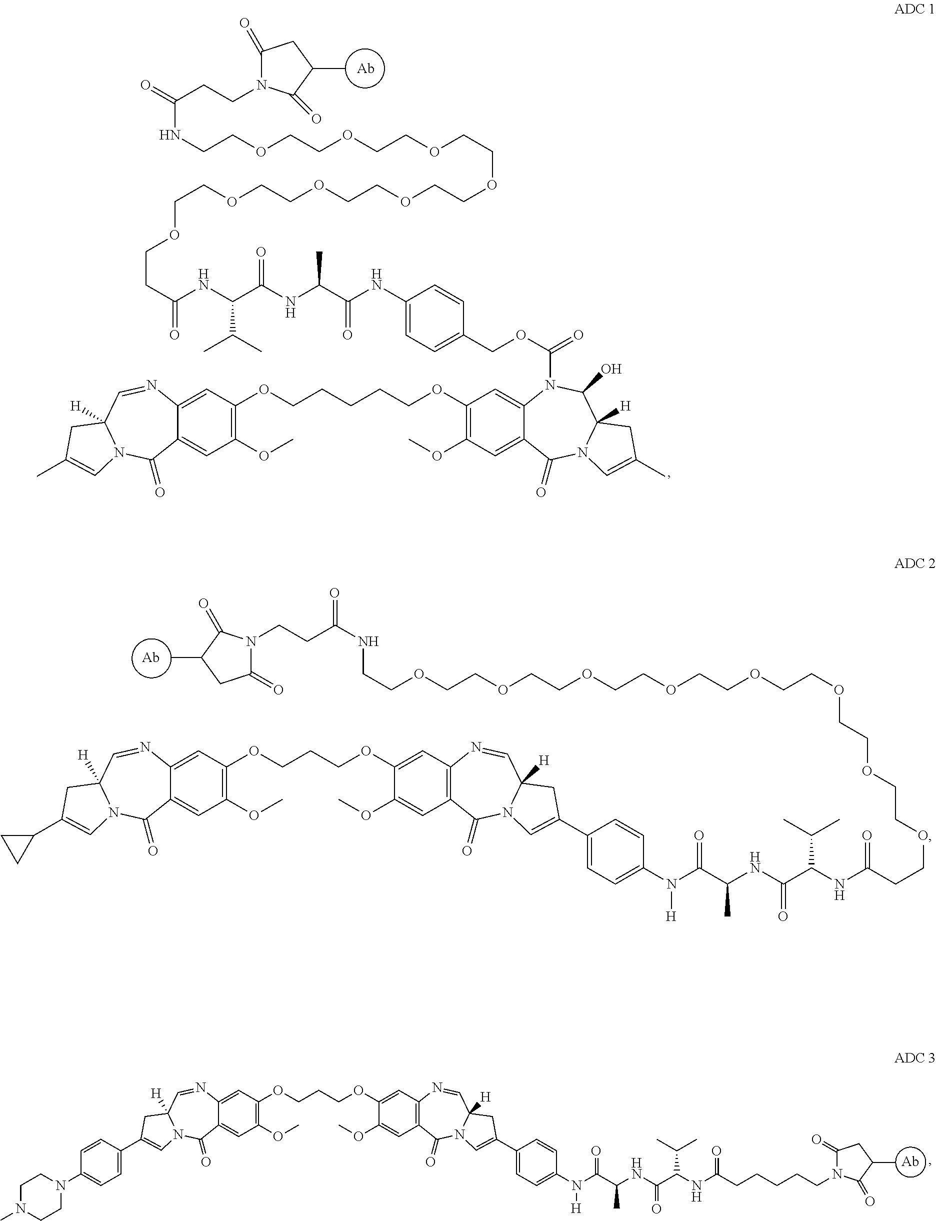 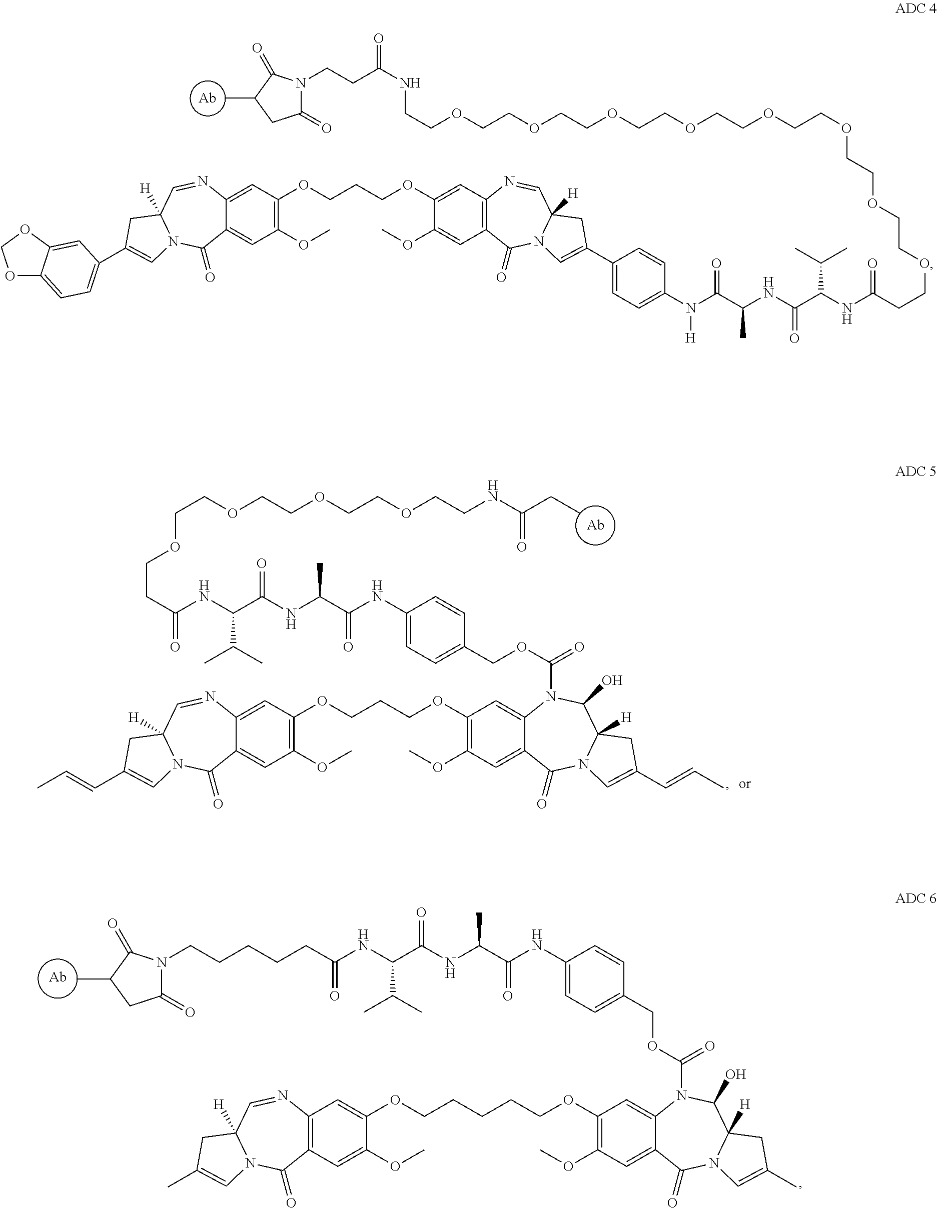
wherein Ab comprises the monoclonal antibody.

42. The antibody drug conjugate of claim 36, wherein the antibody drug conjugate has a drug loading of 2.

43. An antibody drug conjugate that has the structure: 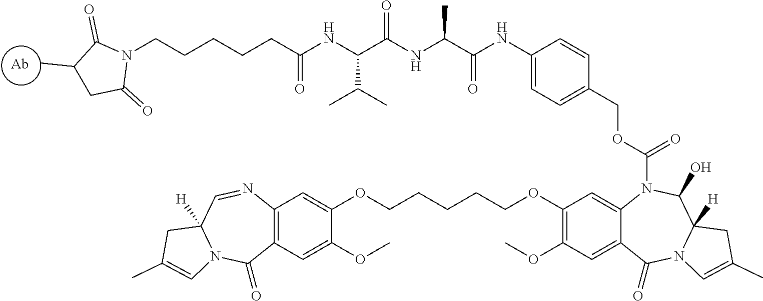
wherein the Ab comprises an antibody comprising a light chain comprising an amino acid sequence set forth as SEQ ID NO: 86 and a heavy chain comprising an amino acid sequence set forth as SEQ ID NO: 89.

44. A pharmaceutical composition comprising:
(a) the antibody drug conjugate of claim 36; and
(b) a pharmaceutically acceptable carrier.

45. The pharmaceutical composition of claim 44, comprising a drug to antibody ratio (DAR) of 2±0.4.

46. The pharmaceutical composition of claim 44, wherein the predominant antibody drug conjugate species is present at a concentration of greater than 70%.

47. A method of (i) treating cancer or (ii) reducing the frequency of tumor initiating cells, comprising administering the antibody drug conjugate of claim 36 to a subject in need thereof.

48. The method of claim 47, wherein the cancer is ovarian cancer, lung adenocarcinoma, or endometrial cancer.

49. A method of making an antibody drug conjugate, comprising conjugating the monoclonal antibody of claim 31 to a cytotoxic agent.

50. A kit comprising:
(a) one or more containers comprising the antibody drug conjugate of claim 36; and
(b) a label or package insert on or associated with the one or more containers, wherein the label or package insert indicates that the antibody drug conjugate is used for (a) treating cancer or (b) reducing the frequency of tumor initiating cells.
